SEQUENCE LISTING (2 sequences)

SEQ ID NO: 1 - protein, 313 aa, Haemophilus paracuniculus
MNLIICHTPLQVLIAEKIMEKFPNEDFYGVMFTYASYHNQKFQHYWQRFSEKCAKSELFIYPQAGRFSLFSALYQFNKKFPQRQFNNIFVASIDDLFVQTFISQLQFNLLCTFDDGTANIVQNSIYYQDSSQSIVRKMANLVLGNKYSTQKLRQLSQKHYSIYPNMPNIVENIELVSLSNSVEQNSAKFSEKPPLALLLGQPLYEDNQQNIALVERVVEQFGIELYLPHPRESFKTDKVKYIETPLIFEDYISQQSQQQKYRIYTYFSGAVLNVLNNPNIEVISIKPNLTNPTYLACYPIFEKFRVKVIELDK

SEQ ID NO: 2 - protein, 223 aa, Pasteurella multocida
MTNIAIIPARAGSKGIPDKNLQPVGGHSLIGRAILAAKNADVFDMIVVTSDGDNILREAEKYGALALKRPAELAQDNSRTIDAILHALESLNIREGTCTLLQPTSPLRDHLDIKNAMDMYVNGGVHSVVSACECEHHPYKAFALSKDHEVLPVREIADFEAARQTLPKMYRANGAIYINDIAQLLKEKYFFIPPLKFYLMPTYRSVDIDVKQDLELAEILSNK

(19) United States
(12) Patent Application Publication
(10) Pub. No.: US 2025/0188503 A1
(43) Pub. Date: Jun. 12, 2025

(54) SIALYLTRANSFERASES FOR THE PRODUCTION OF SIALYLATED OLIGOSACCHARIDES

(71) Applicant: INBIOSE N.V., Zwijnaarde (BE)

(72) Inventors: Joeri Beauprez, Zwijnaarde (BE); Thomas Decoene, Zwijnaarde (BE); Annelies Vercauteren, Zwijnaarde (BE); Tom Verhaeghe, Zwijnaarde (BE)

(21) Appl. No.: 18/845,700

(22) PCT Filed: Mar. 16, 2023
(86) PCT No.: PCT/EP2023/056763

§ 371 Date: Sep. 10, 2024

(30) Foreign Application Priority Data
Mar. 16, 2022 (EP) 22162487.7
Mar. 16, 2022 (LU) LU501672

(51) Int. Cl. C12P 19/02; C12N 9/10; C12N 15/70; C12N 15/74; C12P 17/06; C12P 19/12; C12P 19/18

(52) U.S. Cl. CPC C12P 19/02; C12N 9/1048; C12N 15/70; C12N 15/74; C12P 17/06; C12P 19/12; C12P 19/18

(57) ABSTRACT

The present invention is in the technical field of synthetic biology, metabolic engineering and cell cultivation. The present invention relates to newly identified alpha-2, 6-sialyltransferases having alpha-2,6-sialyltransferase activity on the terminal galactose residue of lacto-N-neotetraose (Gal-β1,4-GlcNAc-β1,3-Gal-β1,4-Glc). The invention also describes methods for the production of a sialylated oligosaccharide using any one of said newly identified alpha-2, 6-sialyltransferases as well as the purification of said sialylated oligosaccharide. The present invention also provides a cell for production of said sialylated oligosaccharide and the use of said cell in a cultivation or incubation.

### FIELD OF THE INVENTION

The present invention is in the technical field of synthetic biology, metabolic engineering and cell cultivation. The present invention relates to newly identified alpha-2,6-sialyltransferases having alpha-2,6-sialyltransferase activity on the terminal galactose residue of lacto-N-neotetraose (Gal-β1,4-GlcNAc-β1,3-Gal-β1,4-Glc). The invention also describes methods for the production of a sialylated oligosaccharide using any one of said newly identified alpha-2,6-sialyltransferases as well as the purification of said sialylated oligosaccharide. The present invention also provides a cell for production of said sialylated oligosaccharide and the use of said cell in a cultivation or incubation.

### BACKGROUND

More than 150 structurally distinct human milk oligosaccharides (HMOs) have been identified to date. Although HMOs represent only a minor amount of total human milk nutrients, their beneficial effects on the development of breast-fed infants became evident over the past decades.

Among the HMOs, sialylated HMOs (SHMOs) were observed to support several beneficial effects as described in the art. Among the sialylated di- and/or oligosaccharides in human milk, 3′sialyllactose, 6′sialyllactose, sialyllacto-N-tetraose a, sialyl lacto-N-tetraose b, sialyllacto-N-tetraose c and disialyllacto-N-tetraose are the most prevalent members.

Sialylated di- and/or oligosaccharides are found to be a complex structure and their chemical or (chemo-)enzymatic syntheses has been proven challenging: there are extensive difficulties, e.g. control of stereochemistry, formation of specific linkages, availability of feedstocks, etc. As a consequence, alternative production methods have been developed, amongst which efforts in metabolic engineering of microorganisms to produce sialylated di- and/or oligosaccharides have been made.

Several sialyltransferases have been identified and characterized to date, from bacterial species e. g. from Neisseria, Campylobacter, Pasteurella, Helicobacter and Photobacterium, as well as from mammals and viruses. Sialyltransferases have been generally classified into six glycosyltransferase (GT) families, based on protein sequence similarities. Sialyltransferases are distinguished due to the glycosidic linkages that they form, e. g. into a-2,3-, a-2,6- and a-2,8-sialyltransferases. All of these sialyltransferases transfer the sialic acid residue from cytidine 5′-monophosphate sialic acid (e. g. CMP-NeuNAc) to a variety of acceptor molecules, usually a galactose (Gal) moiety, an N-acetylgalactosamine (GalNAc) moiety or an N-acetylglucosamine (GlcNAc) moiety or another sialic acid (Sia) moiety. LSTc (sialyllacto-N-tetraose c, Neu5Ac-α2,6-Gal-β1,4-GlcNAc-β1,3-Gal-β1,4-Glc) is a monosialyl derivative of LNnT (lacto-N-neotetraose, Gal-β1,4-GlcNAc-β1,3-Gal-1,4-Glc) carrying a terminal Neu5Ac-α2,6-Gal.

### DESCRIPTION

### Summary of the Invention

It is an object of the present invention to provide for tools and methods by means of which a sialylated oligosaccharide, preferably a 6′sialylated oligosaccharide, more preferably LSTc (sialyllacto-N-tetraose c, Neu5Ac-α2,6-Gal-β1,4-GlcNAc-β1,3-Gal-β1,4-Glc), can be produced, preferably in an efficient, time and cost-effective way and which yields high amounts of the desired oligosaccharide.

According to the invention, this and other objects are achieved by providing newly identified alpha-2,6-sialyltransferases as described herein, each of which can be used in a method for the production of a sialylated oligosaccharide, preferably a 6′sialylated oligosaccharide, more preferably LSTc. Furthermore, any one of said newly identified alpha-2,6-sialyltransferases can be used in a cell for production of a sialylated oligosaccharide, preferably a 6′sialylated oligosaccharide, more preferably LSTc.

More specifically, the invention provides newly identified alpha-2,6-sialyltransferases having alpha-2,6-sialyltransferase activity on the terminal galactose (Gal) residue of lacto-N-neotetraose (LNnT, Gal-β1,4-GlcNAc-β1,3-Gal-β1,4-Glc) and comprising an amino acid sequence that is at least 60.0% identical over a stretch of at least 100 amino acid residues, preferably at least 150 amino acid residues, more preferably at least 200 amino acid residues, to the amino acid sequence as represented by SEQ ID NO 1. The invention also provides methods and a cell for the production of a sialylated oligosaccharide, preferably a 6′sialylated oligosaccharide, more preferably LSTc. The present invention also provides methods for the purification of said sialylated oligosaccharide, preferably a 6′sialylated oligosaccharide, more preferably LSTc. Furthermore, the present invention provides a cell which is metabolically engineered with any one of said newly identified alpha-2,6-sialyltransferases as described herein and which comprises a pathway for the production of said sialylated oligosaccharide, preferably a 6′sialylated oligosaccharide, more preferably LSTc.

### Definitions

The words used in this specification to describe the invention and its various embodiments are to be understood not only in the sense of their commonly defined meanings, but to include by special definition in this specification structure, material or acts beyond the scope of the commonly defined meanings. Thus, if an element can be understood in the context of this specification as including more than one meaning, then its use in a claim must be understood as being generic to all possible meanings supported by the specification and by the word itself.

The various aspects and embodiments of the invention disclosed herein are to be understood not only in the order and context specifically described in this specification, but to include any order and any combination thereof. Each embodiment as identified herein may be combined together unless otherwise indicated. All publications, patents, and patent applications mentioned in this specification are herein incorporated by reference to the same extent as if each individual publication, patent, or patent application was specifically and individually indicated to be incorporated by reference in its entirety.

Whenever the context requires, unless specifically stated otherwise, all words used in the singular number shall be deemed to include the plural and vice versa. Unless defined otherwise, all technical and scientific terms used herein generally have the same meaning as commonly understood by one of ordinary skill in the art to which this invention belongs. Generally, the nomenclature used herein and the laboratory procedures in cell culture, molecular genetics, organic chemistry and nucleic acid chemistry and hybridization described herein are those well-known and commonly employed in the art. Standard techniques are used for nucleic acid and peptide synthesis. Generally, enzymatic reactions and purification steps are performed according to the manufacturer's specifications.

In the specification, there have been disclosed embodiments of the invention, and although specific terms are employed, the terms are used in a descriptive sense only and not for purposes of limitation, the scope of the invention being set forth in the following claims. It must be understood that the illustrated embodiments have been set forth only for the purposes of example and that it should not be taken as limiting the invention. It will be apparent to those skilled in the art that alterations, other embodiments, improvements, details and uses can be made consistent with the letter and spirit of the disclosure herein and within the scope of this disclosure, which is limited only by the claims, construed in accordance with the patent law, including the doctrine of equivalents. In the claims that follow, reference characters used to designate claim steps are provided for convenience of description only, and are not intended to imply any particular order for performing the steps, unless specifically stated otherwise.

Throughout the application, unless explicitly stated otherwise, the features “synthesize”, “synthesized” and “synthesis” are interchangeably used with the features “produce”, “produced” and “production”, respectively. Throughout the application, unless explicitly stated otherwise, the expressions “capable of . . . <verb>” and “capable to . . . <verb>” are preferably replaced with the active voice of said verb and vice versa. For example, the expression “capable of expressing” is preferably replaced with “expresses” and vice versa, i.e. “expresses” is preferably replaced with “capable of expressing”. In this document and in its claims, the verbs “to comprise”, “to have” and “to contain” and their conjugations are used in their non-limiting sense to mean that items following the word are included, but items not specifically mentioned are not excluded. Throughout the application, the verb “to comprise” may be replaced by “to consist” or “to consist essentially of” and vice versa. In addition, the verb “to consist” may be replaced by “to consist essentially of” meaning that a composition as defined herein may comprise additional component(s) than the ones specifically identified, said additional component(s) not altering the unique characteristic of the invention. In this document and in its claims, unless specifically stated otherwise, the verbs “to comprise”, “to have” and “to contain”, and their conjugations, may be replaced by “to consist of” (and its conjugations) or “to consist essentially of” (and its conjugations) and vice versa. In addition, reference to an element by the indefinite article “a” or “an” does not exclude the possibility that more than one of the elements is present, unless the context clearly requires that there be one and only one of the elements. The indefinite article “a” or “an” thus usually means “at least one”.

Throughout the application, unless explicitly stated otherwise, the articles “a” and “an” are preferably replaced by “at least two”, more preferably by “at least three”, even more preferably by “at least four”, even more preferably by “at least five”, even more preferably by “at least six”, most preferably by “at least two”. The word “about” or “approximately” when used in association with a numerical value (e.g. “about 10”) or with a range (e.g. “about x to approximately y”) preferably means that the value or range is interpreted as being as accurate as the method used to measure it. If no error margins are specified, the expression “about” or “approximately” when used in association with a numerical value is interpreted as having the same round-off as the given value. Throughout this document and its claims, unless otherwise stated, the expression “from x to y”, wherein x and y represent numerical values, refers to a range of numerical values wherein x is the lower value of the range and y is the upper value of the range. Herein, x and y are also included in the range.

According to the present invention, the term “polynucleotide(s)” generally refers to any polyribonucleotide or polydeoxyribonucleotide, which may be unmodified RNA or DNA or modified RNA or DNA. “Polynucleotide(s)” include, without limitation, single- and double-stranded DNA, DNA that is a mixture of single- and double-stranded regions or single-, double- and triple-stranded regions, single- and double-stranded RNA, and RNA that is mixture of single- and double-stranded regions, hybrid molecules comprising DNA and RNA that may be single-stranded or, more typically, double-stranded, or triple-stranded regions, or a mixture of single- and double-stranded regions. In addition, “polynucleotide” as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide. As used herein, the term “polynucleotide(s)” also includes DNAs or RNAs as described above that contain one or more modified bases. Thus, DNAs or RNAs with backbones modified for stability or for other reasons are “polynucleotide(s)” according to the present invention. Moreover, DNAs or RNAs comprising unusual bases, such as inosine, or modified bases, such as tritylated bases, are to be understood to be covered by the term “polynucleotides”. It will be appreciated that a great variety of modifications have been made to DNA and RNA that serve many useful purposes known to those of skill in the art. The term “polynucleotide(s)” as it is employed herein embraces such chemically, enzymatically or metabolically modified forms of polynucleotides, as well as the chemical forms of DNA and RNA characteristic of viruses and cells, including, for example, simple and complex cells. The term “polynucleotide(s)” also embraces short polynucleotides often referred to as oligonucleotide(s).

“Polypeptide(s)” refers to any peptide or protein comprising two or more amino acids joined to each other by peptide bonds or modified peptide bonds. “Polypeptide(s)” refers to both short chains, commonly referred to as peptides, oligopeptides and oligomers and to longer chains generally referred to as proteins. Polypeptides may contain amino acids other than the 20 gene encoded amino acids. “Polypeptide(s)” include those modified either by natural processes, such as processing and other post-translational modifications, but also by chemical modification techniques. Such modifications are well described in basic texts and in more detailed monographs, as well as in a voluminous research literature, and they are well known to the skilled person. The same type of modification may be present in the same or varying degree at several sites in a given polypeptide. Furthermore, a given polypeptide may contain many types of modifications. Modifications can occur anywhere in a polypeptide, including the peptide backbone, the amino acid sidechains, and the amino or carboxyl termini. Modifications include, for example, acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphatidylinositol, cross-linking, cyclization, disulphide bond formation, demethylation, formation of covalent cross-links, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, lipid attachment, sulfation, gamma-carboxylation of glutamic acid residues, hydroxylation and ADP-ribosylation, selenoylation, transfer-RNA mediated addition of amino acids to proteins, such as arginylation, and ubiquitination. Polypeptides may be branched or cyclic, with or without branching. Cyclic, branched and branched circular polypeptides may result from post-translational natural processes and may be made by entirely synthetic methods, as well.

The term “polynucleotide encoding a polypeptide” as used herein encompasses polynucleotides that include a sequence encoding a polypeptide of the invention. The term also encompasses polynucleotides that include a single continuous region or discontinuous regions encoding the polypeptide (for example, interrupted by integrated phage or an insertion sequence or editing) together with additional regions that also may contain coding and/or non-coding sequences.

“Isolated” means altered “by the hand of man” from its natural state, i.e. if it occurs in nature, it has been changed or removed from its original environment, or both. For example, a polynucleotide or a polypeptide naturally present in a living organism is not “isolated,” but the same polynucleotide or polypeptide separated from the coexisting materials of its natural state is “isolated”, as the term is employed herein. Similarly, a “synthetic” sequence, as the term is used herein, means any sequence that has been generated synthetically and not directly isolated from a natural source. “Synthesized”, as the term is used herein, means any synthetically generated sequence and not directly isolated from a natural source.

“Recombinant” means genetically engineered DNA prepared by transplanting or splicing genes from one species into the cells of a host organism of a different species. Such DNA becomes part of the host's genetic makeup and is replicated.

The terms “recombinant” or “transgenic” or “metabolically engineered” or “genetically engineered” as used herein with reference to a cell or host cell are used interchangeably and indicates that the cell replicates a heterologous nucleic acid, or expresses a peptide or protein encoded by a heterologous nucleic acid (i.e. a sequence “foreign to said cell” or a sequence “foreign to said location or environment in said cell”). Such cells are described to be transformed with at least one heterologous or exogenous gene or are described to be transformed by the introduction of at least one heterologous or exogenous gene. Metabolically engineered or recombinant or transgenic or genetically engineered cells can contain genes that are not found within the native (non-recombinant) form of the cell. Recombinant cells can also contain genes found in the native form of the cell wherein the genes are modified and re-introduced into the cell by artificial means. The terms also encompass cells that contain a nucleic acid endogenous to the cell that has been modified or its expression or activity has been modified without removing the nucleic acid from the cell; such modifications include those obtained by gene replacement, replacement of a promoter; site-specific mutation; CrispR; riboswitch; recombineering; ssDNA mutagenesis; transposon mutagenesis and related techniques as known to a person skilled in the art. Accordingly, a “recombinant polypeptide” is one which has been produced by a recombinant cell. The terms also encompass cells that have been modified by removing a nucleic acid endogenous to the cell by means of common well-known technologies for a skilled person (like e.g. knocking-out genes).

A “heterologous sequence” or a “heterologous nucleic acid”, as used herein, is one that originates from a source foreign to the particular cell (e.g. from a different species), or, if from the same source, is modified from its original form or place in the genome. Thus, a heterologous nucleic acid operably linked to a promoter is from a source different from that from which the promoter was derived, or, if from the same source, is modified from its original form or place in the genome. The heterologous sequence may be stably introduced, e.g. by transfection, transformation, conjugation or transduction, into the genome of the host microorganism cell, wherein techniques may be applied which will depend on the cell and the sequence that is to be introduced. Various techniques are known to a person skilled in the art and are, e.g. disclosed in Sambrook et al., Molecular Cloning: A Laboratory Manual, 2nd Ed., Cold Spring Harbor Laboratory Press, Cold Spring Harbor, N.Y. (1989). The term “mutant” or “engineered” cell or microorganism as used within the context of the present invention refers to a cell or microorganism which is genetically engineered.

The term “endogenous,” within the context of the present disclosure refers to any polynucleotide, polypeptide or protein sequence that is a natural part of a cell and is occurring at its natural location in the cell chromosome. The term “exogenous” refers to any polynucleotide, polypeptide or protein sequence that originates from outside the cell under study and not a natural part of the cell or that is not occurring at its natural location in the cell chromosome or plasmid.

The term “heterologous” when used in reference to a polynucleotide, gene, nucleic acid, polypeptide, or enzyme refers to a polynucleotide, gene, nucleic acid, polypeptide, or enzyme that is from a source or derived from a source other than the host organism species. In contrast a “homologous” polynucleotide, gene, nucleic acid, polypeptide, or enzyme is used herein to denote a polynucleotide, gene, nucleic acid, polypeptide, or enzyme that is derived from the host organism species. When referring to a gene regulatory sequence or to an auxiliary nucleic acid sequence used for maintaining or manipulating a gene sequence (e.g. a promoter, a 5′ untranslated region, 3′ untranslated region, poly A addition sequence, intron sequence, splice site, ribosome binding site, internal ribosome entry sequence, genome homology region, recombination site, etc.), “heterologous” means that the regulatory sequence or auxiliary sequence is not naturally associated with the gene with which the regulatory or auxiliary nucleic acid sequence is juxtaposed in a construct, genome, chromosome, or episome. Thus, a promoter operably linked to a gene to which it is not operably linked to in its natural state (i.e. in the genome of a non-genetically engineered organism) is referred to herein as a “heterologous promoter,” even though the promoter may be derived from the same species (or, in some cases, the same organism) as the gene to which it is linked.

The term “modified expression” of a gene relates to a change in expression compared to the wild-type expression of said gene in any phase of the production process of the desired sialylated di- and/or oligosaccharide. Said modified expression is either a lower or higher expression compared to the wild-type, wherein the term “higher expression” is also defined as “overexpression” of said gene in the case of an endogenous gene or “expression” in the case of a heterologous gene that is not present in the wild-type strain. Lower expression is obtained by means of common well-known technologies for a skilled person (such as the usage of siRNA, CrispR, CrispRi, riboswitch, recombineering, homologous recombination, ssDNA mutagenesis, RNAi, miRNA, asRNA, mutating genes, knocking-out genes, transposon mutagenesis, etc.) which are used to change the genes in such a way that they are less-able (i.e. statistically significantly ‘less-able’ compared to a functional wild-type gene) or completely unable (such as knocked-out genes) to produce functional final products. The term “riboswitch” as used herein is defined to be part of the messenger RNA that folds into intricate structures that block expression by interfering with translation. Binding of an effector molecule induces conformational change(s) permitting regulated expression post-transcriptionally. Next to changing the gene of interest in such a way that lower expression is obtained as described above, lower expression can also be obtained by changing the transcription unit, the promoter, an untranslated region, the ribosome binding site, the Shine Dalgarno sequence or the transcription terminator. Lower expression or reduced expression can for instance be obtained by mutating one or more base pairs in the promoter sequence or changing the promoter sequence fully to a constitutive promoter with a lower expression strength compared to the wild-type or an inducible promoter which result in regulated expression or a repressible promoter which results in regulated expression.

Overexpression or expression is obtained by means of common well-known technologies for a skilled person (such as the usage of artificial transcription factors, de novo design of a promoter sequence, ribosome engineering, introduction or re-introduction of an expression module at euchromatin, usage of high-copy-number plasmids), wherein said gene is part of an “expression cassette” that relates to any sequence in which a promoter sequence, untranslated region sequence (containing either a ribosome binding sequence, Shine Dalgarno or Kozak sequence), a coding sequence (for instance a sialyltransferase gene sequence) and optionally a transcription terminator is present, and leading to the expression of a functional active protein. Said expression is either constitutive or conditional or regulated or tuneable.

The term “constitutive expression” is defined as expression that is not regulated by transcription factors other than the subunits of RNA polymerase (e.g. the bacterial sigma factors like σ⁷⁰, σ⁵⁴, or related σ-factors and the yeast mitochondrial RNA polymerase specificity factor MTF1 that co-associate with the RNA polymerase core enzyme) under certain growth conditions. Non-limiting examples of such transcription factors are CRP, Lacl, ArcA, Cra, IclR in E. coli, or, Aft2p, Crz1p, Skn7 in Saccharomyces cerevisiae, or, DeoR, GntR, Fur in B. subtilis. These transcription factors bind on a specific sequence and may block or enhance expression in certain growth conditions. The RNA polymerase is the catalytic machinery for the synthesis of RNA from a DNA template. RNA polymerase binds a specific DNA sequence to initiate transcription, for instance via a sigma factor in prokaryotic hosts or via MTF1 in yeasts. Constitutive expression offers a constant level of expression with no need for induction or repression.

The term “regulated expression” is defined as a facultative or regulatory or tuneable expression of a gene that is only expressed upon a certain natural condition of the host (e.g. mating phase of budding yeast, stationary phase of bacteria), as a response to an inducer or repressor such as but not limited to glucose, allo-lactose, lactose, galactose, glycerol, arabinose, rhamnose, fucose, IPTG, methanol, ethanol, acetate, formate, aluminium, copper, zinc, nitrogen, phosphates, xylene, carbon or nitrogen depletion, or substrates or the produced product or chemical repression, as a response to an environmental change (e.g. anaerobic or aerobic growth, oxidative stress, pH shifts, temperature changes like e.g. heat-shock or cold-shock, osmolarity, light conditions, starvation) or dependent on the position of the developmental stage or the cell cycle of said host cell including but not limited to apoptosis and autophagy. Regulated expression allows for control as to when a gene is expressed. The term “inducible expression by a natural inducer” is defined as a facultative or regulatory expression of a gene that is only expressed upon a certain natural condition of the host (e.g. organism being in labour, or during lactation), as a response to an environmental change (e.g. including but not limited to hormone, heat, cold, pH shifts, light, oxidative or osmotic stress/signalling), or dependent on the position of the developmental stage or the cell cycle of said host cell including but not limited to apoptosis and autophagy. The term “inducible expression upon chemical treatment” is defined as a facultative or regulatory expression of a gene that is only expressed upon treatment with a chemical inducer or repressor, wherein said inducer and repressor comprise but are not limited to an alcohol (e.g. ethanol, methanol), a carbohydrate (e.g. glucose, galactose, glycerol, lactose, arabinose, rhamnose, fucose, allo-lactose), metal ions (e.g. aluminium, copper, zinc), nitrogen, phosphates, IPTG, acetate, formate, xylene.

The term “control sequences” refers to sequences recognized by the cells transcriptional and translational systems, allowing transcription and translation of a polynucleotide sequence to a polypeptide. Such DNA sequences are thus necessary for the expression of an operably linked coding sequence in a particular host cell, cell or organism. Such control sequences can be, but are not limited to, promoter sequences, ribosome binding sequences, Shine Dalgarno sequences, Kozak sequences, transcription terminator sequences. The control sequences that are suitable for prokaryotes, for example, include a promoter, optionally an operator sequence, and a ribosome binding site. Eukaryotic cells are known to utilize promoters, polyadenylation signals, and enhancers. DNA for a presequence or secretory leader may be operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it affects the transcription of the sequence; or a ribosome binding site is operably linked to a coding sequence if it is positioned so as to facilitate translation. Said control sequences can furthermore be controlled with external chemicals, such as, but not limited to, IPTG, arabinose, lactose, allo-lactose, rhamnose or fucose via an inducible promoter or via a genetic circuit that either induces or represses the transcription or translation of said polynucleotide to a polypeptide.

Generally, “operably linked” means that the DNA sequences being linked are contiguous, and, in the case of a secretory leader, contiguous and in reading phase. However, enhancers do not have to be contiguous. The term “wild-type” refers to the commonly known genetic or phenotypical situation as it occurs in nature.

The term “modified expression of a protein” as used herein refers to i) higher expression or overexpression of an endogenous protein, ii) expression of a heterologous protein, iii) expression and/or overexpression of a variant protein that has a higher activity compared to the wild-type (i.e. native in the expression host) protein, iv) reduced expression of an endogenous protein or v) expression and/or overexpression of a variant protein that has a reduced activity compared to the wild-type (i.e. native in the expression host) protein. Preferably, the term “modified expression of a protein” as used herein refers to i) higher expression or overexpression of an endogenous protein, ii) expression of a heterologous protein or iii) expression and/or overexpression of a variant protein that has a higher activity compared to the wild-type (i.e. native in the expression host) protein.

The term “modified activity” of a protein relates to a non-native activity of the protein in any phase of the production process of the desired di- and/or sialylated oligosaccharide. The term “non-native”, as used herein with reference to the activity of a protein indicates that the protein has been modified to have an abolished, impaired, reduced, delayed, higher, accelerated or improved activity compared to the native activity of said protein. A modified activity of a protein is obtained by modified expression of said protein or is obtained by expression of a modified, i.e. mutant form of the protein. A mutant form of the protein can be obtained by expression of a mutant form of the gene encoding the protein, e.g. comprising a deletion, an insertion and/or a mutation of one or more nucleotides compared to the native gene sequence. A mutant form of a gene can be obtained by techniques well-known to a person skilled in the art, such as but not limited to site-specific mutation; CrispR; riboswitch; recombineering; ssDNA mutagenesis; transposon mutagenesis.

The term “non-native”, as used herein with reference to a cell producing a sialylated di- and/or oligosaccharide, indicates that the sialylated di- and/or oligosaccharide is i) not naturally produced or ii) when naturally produced not in the same amounts by the cell; and that the cell has been genetically engineered to be able to produce said sialylated di- and/or oligosaccharide or to have a higher production of the sialylated di- and/or oligosaccharide.

As used herein, the term “mammary cell(s)” generally refers to mammalian mammary epithelial cell(s), mammalian mammary-epithelial luminal cell(s), or mammalian epithelial alveolar cell(s), or any combination thereof. As used herein, the term “mammary-like cell(s)” generally refers to mammalian cell(s) having a phenotype/genotype similar (or substantially similar) to natural mammalian mammary cell(s) but is/are derived from mammalian non-mammary cell source(s). Such mammalian mammary-like cell(s) may be engineered to remove at least one undesired genetic component and/or to include at least one predetermined genetic construct that is typical of a mammalian mammary cell. Non-limiting examples of mammalian mammary-like cell(s) may include mammalian mammary epithelial-like cell(s), mammalian mammary epithelial luminal-like cell(s), mammalian non-mammary cell(s) that exhibits one or more characteristics of a cell of a mammalian mammary cell lineage, or any combination thereof. Further non-limiting examples of mammalian mammary-like cell(s) may include mammalian cell(s) having a phenotype similar (or substantially similar) to natural mammalian mammary cell(s), or more particularly a phenotype similar (or substantially similar) to natural mammalian mammary epithelial cell(s). A mammalian cell with a phenotype or that exhibits at least one characteristic similar to (or substantially similar to) a natural mammalian mammary cell or a mammalian mammary epithelial cell may comprise a mammalian cell (e.g. derived from a mammary cell lineage or a non-mammary cell lineage) that exhibits either naturally, or has been engineered to, be capable of expressing at least one milk component.

As used herein, the term “non-mammary cell(s)” may generally include any mammalian cell of non-mammary lineage. In the context of the invention, a non-mammary cell can be any mammalian cell capable of being engineered to express at least one milk component. Non-limiting examples of such non-mammary cell(s) include hepatocyte(s), blood cell(s), kidney cell(s), cord blood cell(s), epithelial cell(s), epidermal cell(s), myocyte(s), fibroblast(s), mesenchymal cell(s), or any combination thereof. In some instances, molecular biology and genome editing techniques can be engineered to eliminate, silence, or attenuate myriad genes simultaneously.

i.e. “Variant(s)” as the term is used herein, is a polynucleotide or polypeptide that differs from a reference polynucleotide or polypeptide respectively but retains essential properties. A typical variant of a polynucleotide differs in nucleotide sequence from another, reference polynucleotide. Changes in the nucleotide sequence of the variant may or may not alter the amino acid sequence of a polypeptide encoded by the reference polynucleotide. Nucleotide changes may result in amino acid substitutions, additions, deletions, fusions and truncations in the polypeptide encoded by the reference sequence, as discussed below. A typical variant of a polypeptide differs in amino acid sequence from another, reference polypeptide. Generally, differences are limited so that the sequences of the reference polypeptide and the variant are closely similar overall and, in many regions, identical. A variant and reference polypeptide may differ in amino acid sequence by one or more substitutions, additions, deletions in any combination. A substituted or inserted amino acid residue may or may not be one encoded by the genetic code. A variant of a polynucleotide or polypeptide may be a naturally occurring such as an allelic variant, or it may be a variant that is not known to occur naturally. Non-naturally occurring variants of polynucleotides and polypeptides may be made by mutagenesis techniques, by direct synthesis, and by other recombinant methods known to the persons skilled in the art.

In some embodiments, the present invention contemplates making functional variants by modifying the structure of an enzyme as used in the present invention. Variants can be produced by amino acid substitution, deletion, addition, or combinations thereof. For instance, it is reasonable to expect that an isolated replacement of a leucine with an isoleucine or valine, an aspartate with a glutamate, a threonine with a serine, or a similar replacement of an amino acid with a structurally related amino acid (e.g. conservative mutations) will not have a major effect on the biological activity of the resulting molecule. Conservative replacements are those that take place within a family of amino acids that are related in their side chains. Whether a change in the amino acid sequence of a polypeptide of the invention results in a functional homolog can be readily determined by assessing the ability of the variant polypeptide to produce a response in cells in a fashion similar to the wild-type polypeptide.

“Fragment”, with respect to a polynucleotide, refers to a clone or any part of a polynucleotide molecule, particularly a part of a polynucleotide that retains a usable, functional characteristic of the full-length polynucleotide molecule. Useful fragments include oligonucleotides and polynucleotides that may be used in hybridization or amplification technologies or in the regulation of replication, transcription or translation. A “polynucleotide fragment” refers to any subsequence of a polynucleotide SEQ ID NO, typically, comprising or consisting of at least about 9, 10, 11, 12 consecutive nucleotides from said polynucleotide SEQ ID NO, for example at least about 30 nucleotides or at least about 50 nucleotides of any of the polynucleotide sequences provided herein. Exemplary fragments can additionally or alternatively include fragments that comprise, consist essentially of, or consist of a region that encodes a conserved family domain of a polypeptide. Exemplary fragments can additionally or alternatively include fragments that comprise a conserved domain of a polypeptide. As such, a fragment of a polynucleotide SEQ ID NO preferably means a nucleotide sequence which comprises or consists of said polynucleotide SEQ ID NO wherein no more than about 200, 150, 100, 50 or 25 consecutive nucleotides are missing, preferably no more than about 50 consecutive nucleotides are missing, and which retains a usable, functional characteristic (e.g. activity) of the full-length polynucleotide molecule which can be assessed by the skilled person through routine experimentation. Alternatively, a fragment of a polynucleotide SEQ ID NO preferably means a nucleotide sequence which comprises or consists of an amount of consecutive nucleotides from said polynucleotide SEQ ID NO and wherein said amount of consecutive nucleotides is at least 50.0%, 60.0%, 70.0%, 80.0%, 81.0%, 82.0%, 83.0%, 84.0%, 85.0%, 86.0%, 87.0%, 88.0%, 89.0%, 90.0%, 91.0%, 92.0%, 93.0%, 94.0%, 95.0%, 95.5%, 96.0%, 96.5%, 97.0%, 97.5%, 98.0%, 98.5%, 99.0%, 99.5%, 100%, preferably at least 80.0%, more preferably at least 85.0%, even more preferably at least 87.0%, even more preferably at least 90.0%, even more preferably at least 95.0%, most preferably at least 97.0%, of the full-length of said polynucleotide SEQ ID NO and retains a usable, functional characteristic (e.g. activity) of the full-length polynucleotide molecule which can be routinely assessed by the skilled person. As such, a fragment of a polynucleotide SEQ ID NO preferably means a nucleotide sequence which comprises or consists of said polynucleotide SEQ ID NO, wherein an amount of consecutive nucleotides is missing and wherein said amount is no more than 50.0%, 40.0%, 30.0% of the full-length of said polynucleotide SEQ ID NO, preferably no more than 20.0%, 15.0%, 10.0%, 9.0%, 8.0%, 7.0%, 6.0%, 5.0%, 4.5%, 4.0%, 3.5%, 3.0%, 2.5%, 2.0%, 1.5%, 1.0%, 0.5%, more preferably no more than 15.0%, even more preferably no more than 10.0%, even more preferably no more than 5.0%, most preferably no more than 2.5%, of the full-length of said polynucleotide SEQ ID NO and wherein said fragment retains a usable, functional characteristic (e.g. activity) of the full-length polynucleotide molecule which can be routinely assessed by the skilled person.

“Fragment”, with respect to a polypeptide, refers to a subsequence of the polypeptide that performs at least one biological function of the intact polypeptide in substantially the same manner, or to a similar extent, as does the intact polypeptide. A “subsequence of the polypeptide” or “a stretch of amino acid residues” as described herein refers to a sequence of contiguous amino acid residues derived from the polypeptide. For example, a polypeptide fragment can comprise a recognizable structural motif or functional domain such as a DNA-binding site or domain that binds to a DNA promoter region, an activation domain, or a domain for protein-protein interactions, and may initiate transcription. Fragments can vary in size from as few as 3 amino acid residues to the full length of the intact polypeptide, for example at least about 10 amino acid residues in length, for example at least about 20 amino acid residues in length, for example at least about 30 amino acid residues in length, for example at least about 100 amino acid residues in length, for example at least about 150 amino acid residues in length, for example at least about 200 amino acid residues in length. As such, a fragment of a polypeptide SEQ ID NO (or UniProt ID) preferably means a polypeptide sequence which comprises or consists of said polypeptide SEQ ID NO (or UniProt ID) wherein no more than about 200, 150, 125, 100, 80, 60, 50, 40, 30, 20 or 15 consecutive amino acid residues are missing, preferably no more than about 100 consecutive amino acid residues are missing, more preferably no more than about 50 consecutive amino acid residues are missing, even more preferably no more than about 40 consecutive amino acid residues are missing, and performs at least one biological function of the intact polypeptide in substantially the same manner, preferably to a similar or greater extent, as does the intact polypeptide which can be routinely assessed by the skilled person. Alternatively, a fragment of a polypeptide SEQ ID NO (or UniProt ID) preferably means a polypeptide sequence which comprises or consists of an amount of consecutive amino acid residues from said polypeptide SEQ ID NO (or UniProt ID) and wherein said amount of consecutive amino acid residues is at least 50.0%, 60.0%, 70.0%, 80.0%, 81.0%, 82.0%, 83.0%, 84.0%, 85.0%, 86.0%, 87.0%, 88.0%, 89.0%, 90.0%, 91.0%, 92.0%, 93.0%, 94.0%, 95.0%, 95.5%, 96.0%, 96.5%, 97.0%, 97.5%, 98.0%, 98.5%, 99.0%, 99.5%, 100%, preferably at least 80.0%, more preferably at least 85.0%, even more preferably at least 87.0%, even more preferably at least 90.0%, even more preferably at least 95.0%, most preferably at least 97.0% of the full-length of said polypeptide SEQ ID NO (or UniProt ID) and that performs at least one biological function of the intact polypeptide in substantially the same manner, preferably to a similar or greater extent, as does the intact polypeptide which can be routinely assessed by the skilled person. As such, a fragment of a polypeptide SEQ ID NO (or UniProt ID) preferably means a polypeptide sequence which comprises or consists of said polypeptide SEQ ID NO (or UniProt ID), wherein an amount of consecutive amino acid residues is missing and wherein said amount is no more than 50.0%, 40.0%, 30.0% of the full-length of said polypeptide SEQ ID NO (or UniProt ID), preferably no more than 20.0%, 15.0%, 10.0%, 9.0%, 8.0%, 7.0%, 6.0%, 5.0%, 4.5%, 4.0%, 3.5%, 3.0%, 2.5%, 2.0%, 1.5%, 1.0%, 0.5%, more preferably no more than 15.0%, even more preferably no more than 10.0%, even more preferably no more than 5.0%, most preferably no more than 2.5%, of the full-length of said polypeptide SEQ ID NO (or UniProt ID) and that performs at least one biological function of the intact polypeptide in substantially the same manner, preferably to a similar or greater extent, as does the intact polypeptide which can be routinely assessed by the skilled person.

Throughout the application, the sequence of a polypeptide can be represented by a SEQ ID NO or alternatively by a UniProt ID. Therefore, the terms “polypeptide SEQ ID NO” and “polypeptide UniProt ID” can be interchangeably used, unless explicitly stated otherwise.

A “functional fragment” of a polypeptide has at least one property or activity of the polypeptide from which it is derived, preferably to a similar or greater extent. A functional fragment can, for example, include a functional domain or conserved domain of a polypeptide. It is understood that a polypeptide or a fragment thereof may have conservative amino acid substitutions which have substantially no effect on the polypeptide's activity. By conservative substitutions is intended substitutions of one hydrophobic amino acid for another or substitution of one polar amino acid for another or substitution of one acidic amino acid for another or substitution of one basic amino acid for another etc. Preferably, by conservative substitutions is intended combinations such as glycine by alanine and vice versa; valine, isoleucine and leucine by methionine and vice versa; aspartate by glutamate and vice versa; asparagine by glutamine and vice versa; serine by threonine and vice versa; lysine by arginine and vice versa; cysteine by methionine and vice versa; and phenylalanine and tyrosine by tryptophan and vice versa.

Homologous sequences as used herein describes those nucleotide sequences that have sequence similarity and encode polypeptides that share at least one functional characteristic such as a biochemical activity. More specifically, the term “functional homolog” as used herein describes those polypeptides that have sequence similarity (in other words, homology) and at the same time have at least one functional similarity such as a biochemical activity (Altenhoff et al., PLoS Comput. Biol. 8 (2012) e1002514).

Homologs can be identified by analysis of nucleotide and polypeptide sequence alignments. For example, performing a query on a database of nucleotide or polypeptide sequences can identify homologs of the nucleotides or polypeptides of interest. Sequence analysis can involve BLAST, Reciprocal BLAST, or PSI-BLAST analysis of non-redundant databases using the amino acid sequence of a reference polypeptide sequence. The amino acid sequence is, in some instances, deduced from the nucleotide sequence. Typically, those polypeptides in the database that have greater than 40% sequence identity to a polypeptide of interest are candidates for further evaluation for suitability as a homologous polypeptide. Amino acid sequence similarity allows for conservative amino acid substitutions, such as substitution of one hydrophobic residue for another or substitution of one polar residue for another or substitution of one acidic amino acid for another or substitution of one basic amino acid for another etc. Preferably, by conservative substitutions is intended combinations such as glycine by alanine and vice versa; valine, isoleucine and leucine by methionine and vice versa; aspartate by glutamate and vice versa; asparagine by glutamine and vice versa; serine by threonine and vice versa; lysine by arginine and vice versa; cysteine by methionine and vice versa; and phenylalanine and tyrosine by tryptophan and vice versa. If desired, manual inspection of such candidates can be carried out in order to narrow the number of candidates to be further evaluated.

A domain can be characterized, for example, by a Pfam (El-Gebali et al., Nucleic Acids Res. 47 (2019) D427-D432), an IPR (InterPro domain) (http://ebi.ac.uk/interpro) (Mitchell et al., Nucleic Acids Res. 47 (2019) D351-D360), a protein fingerprint domain (PRINTS) (Attwood et al., Nucleic Acids Res. 31 (2003) 400-402), a SUBFAM domain (Gough et al., J. Mol. Biol. 313 (2001) 903-919), a TIGRFAM domain (Selengut et al., Nucleic Acids Res. 35 (2007) D260-D264), a Conserved Domain Database (CDD) designation (https://www.ncbi.nlm.nih.gov/cdd) (Lu et al., Nucleic Acids Res. 48 (2020) D265-D268), a PTHR domain (http://www.pantherdb.org) (Mi et al., Nucleic Acids. Res. 41 (2013) D377-D386; Thomas et al., Genome Research 13 (2003) 2129-2141) or a PATRIC identifier or PATRIC DB global family domain (https://www.patricbrc.org/) (Davis et al., Nucleic Acids Res. 48(D1) (2020) D606-D612). Protein or polypeptide sequence information and functional information can be provided by a comprehensive resource for protein sequence and annotation data like e.g. the Universal Protein Resource (UniProt) (www.uniprot.org) (Nucleic Acids Res. 2021, 49(D1), D480-D489). UniProt comprises the expertly and richly curated protein database called the UniProt Knowledgebase (UniProtKB), together with the UniProt Reference Clusters (UniRef) and the UniProt Archive (UniParc). The UniProt identifiers (UniProt ID) are unique for each protein present in the database. Throughout the application, the sequence of a polypeptide is represented by a SEQ ID NO or a UniProt ID. Unless stated otherwise, the UniProt IDs of the proteins described correspond to their sequence version 01 as present in the UniProt Database (www.uniprot.org) version release 2021_03 and consulted on 9 Jun. 2021.

InterPro provides functional analysis of proteins by classifying them into families and predicting domains and important sites. To classify proteins in this way, InterPro uses predictive models, known as signatures, provided by several different databases (referred to as member databases) that make up the InterPro consortium. Protein signatures from these member databases are combined into a single searchable resource, capitalizing on their individual strengths to produce a powerful integrated database and diagnostic tool.

It should be understood for those skilled in the art that for the databases used herein, comprising Pfam 32.0 (released September 2018), CDD v3.17 (released 3 Apr. 2019), eggnogdb 4.5.1 (released September 2016), InterPro 82.0 (released 8 Oct. 2020) and PATRIC 3.6.9 (released March 2020), the content of each database is fixed at each release and is not to be changed. When the content of a specific database is changed, this specific database receives a new release version with a new release date. All release versions for each database with their corresponding release dates and specific content as annotated at these specific release dates are available and known to those skilled in the art.

The terms “identical” or “percent identity” or “% identity” in the context of two or more nucleic acid or polypeptide sequences, refer to two or more sequences or subsequences that are the same or have a specified percentage of nucleotides or amino acid residues that are the same, when compared and aligned for maximum correspondence, as measured using sequence comparison algorithms or by visual inspection. For sequence comparison, one sequence acts as a reference sequence, to which test sequences are compared. When using a sequence comparison algorithm, test and reference sequences are inputted into a computer, subsequence coordinates are designated, if necessary, and sequence algorithm program parameters are designated. The sequence comparison algorithm then calculates the % sequence identity for the test sequence(s) relative to the reference sequence, based on the designated program parameters. The percentage of sequence identity can be, preferably is, determined by alignment of the two sequences and identification of the number of positions with identical residues divided by the number of residues in the shorter of the sequences×100. Percent identity may be calculated globally over the full-length sequence of a given SEQ ID NO, i.e. the reference sequence, resulting in a global % identity score. Alternatively, % identity may be calculated over a partial sequence of the reference sequence, resulting in a local percent identity score. A partial sequence preferably means at least about 50%, 60%, 70%, 80%, 90% or 95% of the full-length reference sequence. In another preferred embodiment, a partial sequence of a reference polypeptide sequence means a stretch of at least 100 amino acid residues up to the total number of amino acid residues of a reference polypeptide sequence. In another more preferred embodiment, a partial sequence of a reference polypeptide sequence means a stretch of at least 150 amino acid residues up to the total number of amino acid residues of a reference polypeptide sequence. In a most preferred embodiment, a partial sequence of a reference polypeptide sequence means a stretch of at least 200 amino acid residues up to the total number of amino acid residues of a reference polypeptide sequence. Using the full-length of the reference sequence in a local sequence alignment results in a global percent identity score between the test and the reference sequence.

Percent identity can be determined using different algorithms like for example BLAST and PSI-BLAST (Altschul et al., 1990, J Mol Biol 215:3, 403-410; Altschul et al., 1997, Nucleic Acids Res 25: 17, 3389-402), the Clustal Omega method (Sievers et al., 2011, Mol. Syst. Biol. 7:539), the MatGAT method (Campanella et al., 2003, BMC Bioinformatics, 4:29) or EMBOSS Needle.

As used herein, a polypeptide comprising or consisting of an amino acid sequence having 60.0% or more sequence identity over a stretch of at least 100 amino acid residues of a reference polypeptide sequence is to be understood as that the amino acid sequence has 60.0%, 61.0%, 62.0%, 63.0%, 64.0%, 65.0%, 66.0%, 67.0%, 68.0%, 69.0%, 70.0%, 71.0%, 72.0%, 73.0%, 74.0%, 75.0%, 76.0%, 77.0%, 78.0%, 79.0%, 80.0%, 81.0%, 82.0%, 83.0%, 84.0%, 85.0%, 86.0%, 87.0%, 88.0%, 89.0%, 90.0%, 91.0%, 91.50%, 92.00%, 92.50%, 93.00%, 93.50%, 94.00%, 94.50%, 95.00%, 95.50%, 96.00%, 96.50%, 97.00%, 97.50%, 98.00%, 98.50%, 99.00%, 99.50%, 99.60%, 99.70%, 99.80%, 99.90%, 100% sequence identity over a stretch of at least 100 amino acid residues of the reference polypeptide sequence.

As used herein, a polypeptide comprising or consisting of an amino acid sequence having 60.0% or more sequence identity over a stretch of at least 150 amino acid residues of a reference polypeptide sequence is to be understood as that the amino acid sequence has 60.0%, 61.0%, 62.0%, 63.0%, 64.0%, 65.0%, 66.0%, 67.0%, 68.0%, 69.0%, 70.0%, 71.0%, 72.0%, 73.0%, 74.0%, 75.0%, 76.0%, 77.0%, 78.0%, 79.0%, 80.0%, 81.0%, 82.0%, 83.0%, 84.0%, 85.0%, 86.0%, 87.0%, 88.0%, 89.0%, 90.0%, 91.0%, 91.50%, 92.00%, 92.50%, 93.00%, 93.50%, 94.00%, 94.50%, 95.00%, 95.50%, 96.00%, 96.50%, 97.00%, 97.50%, 98.00%, 98.50%, 99.00%, 99.50%, 99.60%, 99.70%, 99.80%, 99.90%, 100% sequence identity over a stretch of at least 150 amino acid residues of the reference polypeptide sequence.

As used herein, a polypeptide comprising or consisting of an amino acid sequence having 60.0% or more sequence identity over a stretch of at least 200 amino acid residues of a reference polypeptide sequence is to be understood as that the amino acid sequence has 60.0%, 61.0%, 62.0%, 63.0%, 64.0%, 65.0%, 66.0%, 67.0%, 68.0%, 69.0%, 70.0%, 71.0%, 72.0%, 73.0%, 74.0%, 75.0%, 76.0%, 77.0%, 78.0%, 79.0%, 80.0%, 81.0%, 82.0%, 83.0%, 84.0%, 85.0%, 86.0%, 87.0%, 88.0%, 89.0%, 90.0%, 91.0%, 91.50%, 92.00%, 92.50%, 93.00%, 93.50%, 94.00%, 94.50%, 95.00%, 95.50%, 96.00%, 96.50%, 97.00%, 97.50%, 98.00%, 98.50%, 99.00%, 99.50%, 99.60%, 99.70%, 99.80%, 99.90%, 100% sequence identity over a stretch of at least 200 amino acid residues of the reference polypeptide sequence.

As used herein, a polypeptide comprising or consisting of an amino acid sequence having 60.0% or more sequence identity to the full-length sequence of a reference polypeptide sequence is to be understood as that the amino acid sequence has 60.0%, 61.0%, 62.0%, 63.0%, 64.0%, 65.0%, 66.0%, 67.0%, 68.0%, 69.0%, 70.0%, 71.0%, 72.0%, 73.0%, 74.0%, 75.0%, 76.0%, 77.0%, 78.0%, 79.0%, 80.0%, 81.0%, 82.0%, 83.0%, 84.0%, 85.0%, 86.0%, 87.0%, 88.0%, 89.0%, 90.0%, 91.0%, 91.50%, 92.00%, 92.50%, 93.00%, 93.50%, 94.00%, 94.50%, 95.00%, 95.50%, 96.00%, 96.50%, 97.00%, 97.50%, 98.00%, 98.50%, 99.00%, 99.50%, 99.60%, 99.70%, 99.80%, 99.90%, 100% sequence identity to the full-length of the amino acid sequence of the reference polypeptide sequence.

Throughout the application, unless explicitly specified otherwise, a polypeptide comprising, consisting of or having an amino acid sequence having 60.0% or more sequence identity to the full-length amino acid sequence of a reference polypeptide, usually indicated with a SEQ ID NO or UniProt ID, preferably has 60.0%, 65.0%, 70.0%, 75.0%, 80.0%, 85.0%, 90.0%, 91.0%, 92.0%, 93.0%, 94.0%, 95.0%, 96.0%, 97.0%, 98.0% or 99.0%, more preferably has at least 65.0%, even more preferably has at least 70.0%, even more preferably has at least 80.0%, even more preferably has at least 85.0%, most preferably has at least 90.0%, sequence identity to the full length reference sequence. Additionally, unless explicitly specified otherwise, a polynucleotide sequence comprising, consisting of or having a nucleotide sequence having 60.0% or more sequence identity to the full-length nucleotide sequence of a reference polynucleotide sequence, usually indicated with a SEQ ID NO, preferably has 60.0%, 65.0%, 70.0%, 75.0%, 80.0%, 85.0%, 90.0%, 91.0%, 92.0%, 93.0%, 94.0%, 95.0%, 96.0%, 97.0%, 98.0% or 99.0%, more preferably has at least 65.0%, even more preferably has at least 70.0%, even more preferably has at least 80.0%, even more preferably has at least 85.0%, most preferably has at least 90.0% sequence identity to the full-length reference polynucleotide sequence.

For the purposes of this invention, percent identity is determined using MatGAT2.01 (Campanella et al., 2003, BMC Bioinformatics 4:29). The following default parameters for protein are employed: (1) Gap cost Existence: 12 and Extension: 2; (2) The Matrix employed was BLOSUM50.

The terms “sialic acid”, “Neu5Ac”, “N-acetylneuraminate”, “N-acylneuraminate”, “N-acetylneuraminic acid” are used interchangeably and refer to an acidic sugar with a nine-carbon backbone.

Neu5Ac is also known as 5-acetamido-3,5-dideoxy-D-glycero-D-galacto-non-2-ulopyranosonic acid, D-glycero-5-acetamido-3,5-dideoxy-D-galacto-non-2-ulo-pyranosonic acid, 5-(acetylamino)-3,5-dideoxy-D-glycero-D-galacto-2-nonulopyranosonic acid, 5-(acetylamino)-3,5-dideoxy-D-glycero-D-galacto-2-nonulosonic acid, 5-(acetylamino)-3,5-dideoxy-D-glycero-D-galacto-non-2-nonulosonic acid or 5-(acetylamino)-3,5-dideoxy-D-glycero-D-galacto-non-2-ulopyranosonic acid and has C11H19NO9 as molecular formula. The term “glycosyltransferase” as used herein refers to an enzyme capable to catalyse the transfer of a sugar moiety of a donor to a specific acceptor, forming glycosidic bonds. Said donor can be a precursor as defined herein. A classification of glycosyltransferases using nucleotide diphospho-sugar, nucleotide monophospho-sugar and sugar phosphates and related proteins into distinct sequence-based families has been described (Campbell et al., Biochem. J. 326, 929-939 (1997)) and is available on the CAZy (CArbohydrate-Active EnZymes) website (www.cazy.org).

As used herein the glycosyltransferase can be selected from the list comprising but not limited to: fucosyltransferases, sialyltransferases, galactosyltransferases, glucosyltransferases, mannosyltransferases, N-acetylglucosaminyltransferases, N-acetylgalactosaminyltransferases, N-acetylmannosaminyltransferases, xylosyltransferases, glucuronyltransferases, galacturonyltransferases, glucosaminyltransferases, N-glycolylneuraminyltransferases, rhamnosyltransferases, N-acetylrhamnosyltransferases, UDP-4-amino-4,6-dideoxy-N-acetyl-beta-L-altrosamine transaminases, UDP-N-acetylglucosamine enolpyruvyl transferases and fucosaminyltransferases.

Sialyltransferases are glycosyltransferases that transfer a sialic acid (like Neu5Ac) from a donor (like CMP-Neu5Ac) onto an acceptor. Sialyltransferases comprise alpha-2,3-sialyltransferases, alpha-2,6-sialyltransferases and alpha-2,8-sialyltransferases that catalyse the transfer of a sialic acid onto an acceptor via alpha-glycosidic bonds. Sialyltransferases can be found but are not limited to the GT29, GT42, GT80 and GT97 CAZy families.

The terms “alpha-2,6-sialyltransferase”, “alpha 2,6 sialyltransferase”, “6-sialyltransferase”, “α-2,6-sialyltransferase”, “α 2,6 sialyltransferase”, “6 sialyltransferase”, “6-ST” or “6ST” as used in the present invention, are used interchangeably and refer to a glycosyltransferase that catalyzes the transfer of sialic acid from the donor CMP-Neu5Ac, to the acceptor molecule in an alpha-2,6-linkage.

The term “monosaccharide” as used herein refers to a sugar that is not decomposable into simpler sugars by hydrolysis, is classed either an aldose or ketose, and contains one or more hydroxyl groups per molecule. Monosaccharides are saccharides containing only one simple sugar. Examples of monosaccharides comprise Hexose, D-Glucopyranose, D-Galactofuranose, D-Galactopyranose, L-Galactopyranose, D-Mannopyranose, D-Allopyranose, L-Altropyranose, D-Gulopyranose, L-Idopyranose, D-Talopyranose, D-Ribofuranose, D-Ribopyranose, D-Arabinofuranose, D-Arabinopyranose, L-Arabinofuranose, L-Arabinopyranose, D-Xylopyranose, D-Lyxopyranose, D-Erythrofuranose, D-Threofuranose, Heptose, L-glycero-D-manno-Heptopyranose (LDmanHep), D-glycero-D-manno-Heptopyranose (DDmanHep), 6-Deoxy-L-altropyranose, 6-Deoxy-D-gulopyranose, 6-Deoxy-D-talopyranose, 6-Deoxy-D-galactopyranose, 6-Deoxy-L-galactopyranose, 6-Deoxy-D-mannopyranose, 6-Deoxy-L-mannopyranose, 6-Deoxy-D-glucopyranose, 2-Deoxy-D-arabino-hexose, 2-Deoxy-D-erythro-pentose, 2,6-Dideoxy-D-arabino-hexopyranose, 3,6-Dideoxy-D-arabino-hexopyranose, 3,6-Dideoxy-L-arabino-hexopyranose, 3,6-Dideoxy-D-xylo-hexopyranose, 3,6-Dideoxy-D-ribo-hexopyranose, 2,6-Dideoxy-D-ribo-hexopyranose, 3,6-Dideoxy-L-xylo-hexopyranose, 2-Amino-2-deoxy-D-glucopyranose, 2-Amino-2-deoxy-D-galactopyranose, 2-Amino-2-deoxy-D-mannopyranose, 2-Amino-2-deoxy-D-allopyranose, 2-Amino-2-deoxy-L-altropyranose, 2-Amino-2-deoxy-D-gulopyranose, 2-Amino-2-deoxy-L-idopyranose, 2-Amino-2-deoxy-D-talopyranose, 2-Acetamido-2-deoxy-D-glucopyranose, 2-Acetamido-2-deoxy-D-galactopyranose, 2-Acetamido-2-deoxy-D-mannopyranose, 2-Acetamido-2-deoxy-D-allopyranose, 2-Acetamido-2-deoxy-L-altropyranose, 2-Acetamido-2-deoxy-D-gulopyranose, 2-Acetamido-2-deoxy-L-idopyranose, 2-Acetamido-2-deoxy-D-talopyranose, 2-Acetamido-2,6-dideoxy-D-galactopyranose, 2-Acetamido-2,6-dideoxy-L-galactopyranose, 2-Acetamido-2,6-dideoxy-L-mannopyranose, 2-Acetamido-2,6-dideoxy-D-glucopyranose, 2-Acetamido-2,6-dideoxy-L-altropyranose, 2-Acetamido-2,6-dideoxy-D-talopyranose, D-Glucopyranuronic acid, D-Galactopyranuronic acid, D-Mannopyranuronic acid, D-Allopyranuronic acid, L-Altropyranuronic acid, D-Gulopyranuronic acid, L-Gulopyranuronic acid, L-ldopyranuronic acid, D-Talopyranuronic acid, sialic acid, 5-Amino-3,5-dideoxy-D-glycero-D-galacto-non-2-ulosonic acid, 5-Acetamido-3,5-dideoxy-D-glycero-D-galacto-non-2-ulosonic acid, 5-Glycolylamido-3,5-dideoxy-D-glycero-D-galacto-non-2-ulosonic acid, Erythritol, Arabinitol, Xylitol, Ribitol, Glucitol, Galactitol, Mannitol, D-ribo-Hex-2-ulopyranose, D-arabino-Hex-2-ulofuranose (D-fructofuranose), D-arabino-Hex-2-ulopyranose, L-xylo-Hex-2-ulopyranose, D-lyxo-Hex-2-ulopyranose, D-threo-Pent-2-ulopyranose, D-altro-Hept-2-ulopyranose, 3-C-(Hydroxymethyl)-D-erythofuranose, 2,4,6-Trideoxy-2,4-diamino-D-glucopyranose, 6-Deoxy-3-O-methyl-D-glucose, 3-O-Methyl-D-rhamnose, 2,6-Dideoxy-3-methyl-D-ribo-hexose, 2-Amino-3-O—[(R)-1-carboxyethyl]-2-deoxy-D-glucopyranose, 2-Acetamido-3-O—[(R)-carboxyethyl]-2-deoxy-D-glucopyranose, 2-Glycolylamido-3-O—[(R)-1-carboxyethyl]-2-deoxy-D-glucopyranose, 3-Deoxy-D-lyxo-hept-2-ulopyranosaric acid, 3-Deoxy-D-manno-oct-2-ulopyranosonic acid, 3-Deoxy-D-glycero-D-galacto-non-2-ulopyranosonic acid, 5,7-Diamino-3,5,7,9-tetradeoxy-L-glycero-L-manno-non-2-ulopyranosonic acid, 5,7-Diamino-3,5,7,9-tetradeoxy-L-glycero-L-altro-non-2-ulopyranosonic acid, 5,7-Diamino-3,5,7,9-tetradeoxy-D-glycero-D-galacto-non-2-ulopyranosonic acid, 5,7-Diamino-3,5,7,9-tetradeoxy-D-glycero-D-talo-non-2-ulopyranosonic acid, 2-acetamido-2,6-dideoxy-L-arabino-4-hexulose, 2-acetamido-2,6-dideoxy-L-lyxo-4-hexulose, N-acetyl-L-rhamnosamine, N-acetyl-D-fucosamine, N-acetyl-L-pneumosamine, N-acetylmuramic acid, N-acetyl-L-quinovosamine, glucose (Glc), galactose (Gal), N-acetylglucosamine (GlcNAc), glucosamine (Glcn), mannose (Man), xylose (Xyl), N-acetylmannosamine (ManNAc), N-glycolylneuraminic acid, N-acetylgalactosamine (GalNAc), galactosamine (Galn), fucose (Fuc), rhamnose (Rha), glucuronic acid, gluconic acid, fructose (Fru) and polyols. With the term polyol is meant an alcohol containing multiple hydroxyl groups. For example, glycerol, sorbitol, or mannitol.

The term “phosphorylated monosaccharide” as used herein refers to one of the above listed monosaccharides which is phosphorylated. Examples of phosphorylated monosaccharides include but are not limited to glucose-1-phosphate, glucose-6-phosphate, glucose-1,6-bisphosphate, galactose-1-phosphate, fructose-6-phosphate, fructose-1,6-bisphosphate, fructose-1-phosphate, glucosamine-1-phosphate, glucosamine-6-phosphate, N-acetylglucosamine-1-phosphate, mannose-1-phosphate, mannose-6-phosphate or fucose-1-phosphate. Some, but not all, of these phosphorylated monosaccharides are precursors or intermediates for the production of activated monosaccharide.

The terms “activated monosaccharide”, “nucleotide-activated sugar”, “nucleotide-sugar”, “activated sugar”, “nucleoside” or “nucleotide donor” are used herein interchangeably and refer to activated forms of monosaccharides. Examples of activated monosaccharides include but are not limited to UDP-N-acetylglucosamine (UDP-GlcNAc), UDP-N-acetylgalactosamine (UDP-GalNAc), UDP-N-acetylmannosamine (UDP-ManNAc), UDP-glucose (UDP-Glc), UDP-galactose (UDP-Gal), GDP-mannose (GDP-Man), UDP-glucuronate, UDP-galacturonate, UDP-2-acetamido-2,6-dideoxy-L-arabino-4-hexulose, UDP-2-acetamido-2,6-dideoxy-L-lyxo-4-hexulose, UDP-N-acetyl-L-rhamnosamine (UDP-L-RhaNAc or UDP-2-acetamido-2,6-dideoxy-L-mannose), dTDP-N-acetylfucosamine, UDP-N-acetylfucosamine (UDP-L-FucNAc or UDP-2-acetamido-2,6-dideoxy-L-galactose), UDP-N-acetyl-L-pneumosamine (UDP-L-PneNAC or UDP-2-acetamido-2,6-dideoxy-L-talose), UDP-N-acetylmuramic acid, UDP-N-acetyl-L-quinovosamine (UDP-L-QuiNAc or UDP-2-acetamido-2,6-dideoxy-L-glucose), GDP-L-quinovose, CMP-sialic acid (CMP-Neu5Ac or CMP-N-acetylneuraminic acid), GDP-fucose (GDP-Fuc), GDP-rhamnose and UDP-xylose. Nucleotide-sugars act as glycosyl donors in glycosylation reactions. Glycosylation reactions are reactions that are catalysed by glycosyltransferases.

The term “disaccharide” as used herein refers to a saccharide polymer containing two simple sugars, i.e. monosaccharides. Such disaccharides contain monosaccharides preferably selected from the list of monosaccharides as used herein above. Examples of disaccharides comprise lactose (Gal-b1,4-Glc), lacto-N-biose (Gal-b1,3-GlcNAc), N-acetyllactosamine (Gal-b1,4-GlcNAc), LacDiNAc (GalNAc-b1,4-GlcNAc), N-acetylgalactosaminylglucose (GalNAc-b1,4-Glc), Neu5Ac-α2,3-Gal, Neu5Ac-α2,6-Gal and fucopyranosyl-(1-4)-N-glycolylneuraminic acid (Fuc-(1-4)-Neu5Gc).

“Oligosaccharide” as the term is used herein and as generally understood in the state of the art, refers to a saccharide polymer containing a small number, typically three to twenty, preferably three to ten, of simple sugars, i.e. monosaccharides. Preferably the oligosaccharide as described herein contains monosaccharides selected from the list as used herein above. The oligosaccharide as used in the present invention can be a linear structure or can include branches. The linkage (e.g. glycosidic linkage, galactosidic linkage, glucosidic linkage, etc.) between two sugar units can be expressed, for example, as 1,4, 1->4, or (1-4), used interchangeably herein. For example, the terms “Gal-b1,4-Glc”, “Gal-β1,4-Glc”, “b-Gal-(1->4)-Glc”, “β-Gal-(1->4)-Glc”, “Galbeta1-4-Glc”, “Gal-b(1-4)-Glc” and “Gal-β(1-4)-Glc” have the same meaning, i.e. a beta-glycosidic bond links carbon-1 of galactose (Gal) with the carbon-4 of glucose (Glc). Each monosaccharide can be in the cyclic form (e.g. pyranose or furanose form). Linkages between the individual monosaccharide units may include alpha 1->2, alpha 1->3, alpha 1->4, alpha 1->6, alpha 2->1, alpha 2->3, alpha 2->4, alpha 2->6, beta 1->2, beta 1->3, beta 1->4, beta 1->6, beta 2->1, beta 2->3, beta 2->4, and beta 2->6. An oligosaccharide can contain both alpha- and beta-glycosidic bonds or can contain only alpha-glycosidic or only beta-glycosidic bonds. The term “polysaccharide” refers to a compound consisting of a large number, typically more than twenty, of monosaccharides linked glycosidically.

Examples of oligosaccharides include but are not limited to Lewis-type antigen oligosaccharides, mammalian (including human) milk oligosaccharides, O-antigen, enterobacterial common antigen (ECA), the glycan chain present in lipopolysaccharides (LPS), the oligosaccharide repeats present in capsular polysaccharides, peptidoglycan (PG), amino-sugars and antigens of the human ABO blood group system, neutral (non-charged) oligosaccharides, negatively charged oligosaccharides, fucosylated oligosaccharides, sialylated oligosaccharides, N-acetylglucosamine containing oligosaccharides, N-acetyllactosamine containing oligosaccharides, lacto-N-biose containing oligosaccharides, lactose containing oligosaccharides, non-fucosylated neutral (non-charged) oligosaccharides, N-acetyllactosamine containing fucosylated oligosaccharides, N-acetyllactosamine non-fucosylated oligosaccharides, lacto-N-biose containing fucosylated oligosaccharides, lacto-N-biose containing non-fucosylated oligosaccharides, N-acetyllactosamine containing negatively charged oligosaccharides, lacto-N-biose containing negatively charged oligosaccharides, animal oligosaccharides, preferably selected from the group consisting of N-glycans and O-glycans, and plant oligosaccharides, preferably selected from the group consisting of N-glycans and O-glycans.

As used herein, “mammalian milk oligosaccharide” refers to oligosaccharides such as but not limited to 3-fucosyllactose, 2′-fucosyllactose, 6-fucosyllactose, 2′,3-difucosyllactose, 2′,2-difucosyllactose, 3,4-difucosyllactose, 6′-sialyllactose, 3′-sialyllactose, 3,6-disialyllactose, 6,6′-disialyllactose, 8,3-disialyllactose, 3,6-disialyllacto-N-tetraose, lactodifucotetraose, lacto-N-tetraose, lacto-N-neotetraose, lacto-N-fucopentaose II, lacto-N-fucopentaose I, lacto-N-fucopentaose Ill, lacto-N-fucopentaose V, lacto-N-fucopentaose VI, sialyllacto-N-tetraose c, sialyllacto-N-tetraose b, sialyllacto-N-tetraose a, lacto-N-difucohexaose I, lacto-N-difucohexaose II, lacto-N-hexaose, lacto-N-neohexaose, para-lacto-N-hexaose, monofucosylmonosialyllacto-N-tetraose c, monofucosyl para-lacto-N-hexaose, monofucosyllacto-N-hexaose Ill, isomeric fucosylated lacto-N-hexaose Ill, isomeric fucosylated lacto-N-hexaose I, sialyllacto-N-hexaose, sialyllacto-N-neohexaose II, difucosyl-para-lacto-N-hexaose, difucosyllacto-N-hexaose, difucosyllacto-N-hexaose a, difucosyllacto-N-hexaose c, galactosylated chitosan, fucosylated oligosaccharides, neutral oligosaccharide and/or sialylated oligosaccharides.

As used herein, a ‘sialylated oligosaccharide’ is to be understood as a negatively charged sialic acid containing oligosaccharide, i.e. an oligosaccharide having a sialic acid residue. It has an acidic nature. Some examples are 3-SL (3′-sialyllactose or 3′SL or Neu5Ac-a2,3-Gal-b1,4-Glc), 3′-sialyllactosamine, 6-SL (6′sialyllactose, 6′-sialyllactose or 6′SL or Neu5Ac-a2,6-Gal-b1,4-Glc), 3,6-disialyllactose (Neu5Ac-a2,3-(Neu5Ac-a2,6)-Gal-b1,4-Glc), 6,6′-disialyllactose (Neu5Ac-a2,6-Gal-b1,4-(Neu5Ac-a2,6)-Glc), 8,3-disialyllactose (Neu5Ac-a2,8-Neu5Ac-a2,3-Gal-b1,4-Glc), 6′-sialyllactosamine, oligosaccharides comprising 6′sialyllactose (also known as 6′sialyllactose, 6′SL and 6′-SL), SGG hexasaccharide (Neu5Acα-2,3Galß-1,3GalNacβ-1,3Galα-1,4Galβ-1,4Gal), sialylated tetrasaccharide (Neu5Acα-2,3Galβ-1,4GlcNacß-14GlcNAc), pentasaccharide LSTD (Neu5Acα-2,3Galβ-1,4GlcNacβ-1,3Galβ-1,4Glc), sialylated lacto-N-triose, sialylated lacto-N-tetraose, sialyllacto-N-neotetraose, LSTc (Neu5Ac-α2,6-Gal-β1,4-GlcNAc-β1,3-Gal-β1,4-Glc), monosialyllacto-N-hexaose, disialyllacto-N-hexaose I, monosialyllacto-N-neohexaose I, monosialyllacto-N-neohexaose II, disialyllacto-N-neohexaose, disialyllacto-N-tetraose, disialyllacto-N-hexaose II, sialyllacto-N-tetraose a, disialyllacto-N-hexaose I, sialyllacto-N-tetraose b, 3′-sialyl-3-fucosyllactose, disialomonofucosyllacto-N-neohexaose, monofucosylmonosialyllacto-N-octaose (sialyl Lea), sialyllacto-N-fucohexaose II, disialyllacto-N-fucopentaose II, monofucosyldisialyllacto-N-tetraose and oligosaccharides bearing one or several sialic acid residu(s), including but not limited to: oligosaccharide moieties of the gangliosides selected from GM3 (3′sialyllactose, Neu5Acα-2,3Galβ-4Glc) and oligosaccharides comprising the GM3 motif, GD3 Neu5Acα-2,8Neu5Acα-2,3Galβ-1,4Glc GT3 (Neu5Acα-2,8Neu5Acα-2,8Neu5Acα-2,3Galβ-1,4Glc); GM2 GalNAcβ-1,4(Neu5Acα-2,3)Galβ-1,4Glc, GM1 Galβ-1,3GalNAcβ-1,4(Neu5Acα-2,3)Galβ-1,4Glc, GD1a Neu5Acα-2,3Galβ-1,3GalNAcβ-1,4(Neu5Acα-2,3)Galβ-1,4Glc, GT1a Neu5Acα-2,8Neu5Acα-2,3Galβ-1,3GalNAcβ-1,4(Neu5Acα-2,3)Galβ-1,4Glc, GD2 GalNAcβ-1,4(Neu5Acα-2,8Neu5Acα2,3)Galβ-1,4Glc, GT2 GalNAcβ-1,4(Neu5Acα-2,8Neu5Acα-2,8Neu5Acα2,3)Galβ-1,4Glc, GD1b, Galβ-1,3GalNAcβ-1,4(Neu5Acα-2,8Neu5Acα2,3)Galβ-1,4Glc, GT1b Neu5Acα-2,3Galβ-1,3GalNAcβ-1,4(Neu5Acα-2,8Neu5Acα2,3)Galβ-1,4Glc, GQ1b Neu5Acα-2,8Neu5Acα-2,3Galβ-1,3GalNAc 6-1,4(Neu5Acα-2,8Neu5Acα2,3)Galβ-1,4Glc, GT1c Galβ-1,3GalNAcβ-1,4(Neu5Acα-2,8Neu5Acα-2,8Neu5Acα2,3)Galβ-1,4Glc, GQ1c Neu5Acα-2,3Galβ-1,3GalNAc 6-1,4(Neu5Acα-2,8Neu5Acα-2,8Neu5Acα2,3)Galβ-1,4Glc, GP1c Neu5Acα-2,8Neu5Acα-2,3Galβ-1,3GalNAc 6-1,4(Neu5Acα-2,8Neu5Acα-2,8Neu5Acα2,3)Galβ-1,4Glc, GD1a Neu5Acα-2,3Galβ-1,3(Neu5Acα-2,6)GalNAcß-1,4Galβ-1,4Glc, Fucosyl-GM1 Fuca-1,2Galβ-1,3GalNAcß-1,4(Neu5Acα-2,3)Gal 6-1,4Glc; all of which may be extended to the production of the corresponding gangliosides by reacting the above oligosaccharide moieties with ceramide or synthetizing the above oligosaccharides on a ceramide.

As used herein, a ‘6′sialylated oligosaccharide’ is to be understood as a negatively charged sialic acid containing oligosaccharide comprising an oligosaccharide or disaccharide which is alpha-2,6-glycosidically linked to a sialic acid residue.

A ‘neutral oligosaccharide‘ or a’non-charged oligosaccharide’ as used herein and as generally understood in the state of the art is an oligosaccharide that has no negative charge originating from a carboxylic acid group. Examples of such neutral oligosaccharide are 2′-fucosyllactose (2′FL), 3-fucosyllactose (3FL), 2, 3-difucosyllactose (diFL), lacto-N-triose II (LN3), lacto-N-tetraose (LNT), lacto-N-neotetraose (LNnT), lacto-N-fucopentaose I (LNFP 1), lacto-N-neofucopentaose I (LNnFP 1), lacto-N-fucopentaose II (LNFP 11), lacto-N-fucopentaose 11 (LNFP 111), lacto-N-fucopentaose V (LNFP V), lacto-N-fucopentaose VI, lacto-N-neofucopentaose V (LNnFP V), lacto-N-difucohexaose I (LNDFH 1), lacto-N-difucohexaose II (LNDFH 11), 6′-galactosyllactose, 3′-galactosyllactose, lacto-N-hexaose, lacto-N-neohexaose, para-lacto-N-hexaose, para-lacto-N-neohexaose, difucosyl-lacto-N-hexaose and difucosyl-lacto-N-neohexaose, difucosyl-lacto-N-neohexaose (LNnDFH 11), difucosyl-para-lacto-N-neohexaose, trifucosyllacto-N-hexaose, para-lacto-N-fucohexaose and lacto-N-trifucoheptaose.

Mammalian milk oligosaccharides or MMOs comprise oligosaccharides present in milk found in any phase during lactation including colostrum milk from humans (i.e. human milk oligosaccharides or HMOs) and mammals including but not limited to cows (Bos taurus), sheep (Ovis aries), goats (Capra aegagrus hircus), bactrian camels (Camelus bactrianus), horses (Equus ferus caballus), pigs (Sus scropha), dogs (Canis lupus familiaris), ezo brown bears (Ursus arctos yesoensis), polar bear (Ursus maritimus), Japanese black bears (Ursus thibetanus japonicus), striped skunks (Mephitis mephitis), hooded seals (Cystophora cristata), Asian elephants (Elephas maximus), African elephant (Loxodonta africana), giant anteater (Myrmecophaga tridactyla), common bottlenose dolphins (Tursiops truncates), northern minke whales (Balaenoptera acutorostrata), tammar wallabies (Macropus eugenii), red kangaroos (Macropus rufus), common brushtail possum (Trichosurus vulpecula), koalas (Phascolarctos cinereus), eastern quolls (Dasyurus viverrinus), platypus (Ornithorhynchus anatinus). As used herein, “mammalian milk oligosaccharide” refers to oligosaccharides such as but not limited to 3-fucosyllactose, 2′-fucosyllactose, 6-fucosyllactose, 2′,3-difucosyllactose, 2′,2-difucosyllactose, 3,4-difucosyllactose, 6′-sialyllactose, 3′-sialyllactose, 3,6-disialyllactose, 6,6′-disialyllactose, 8,3-disialyllactose, 3,6-disialyllacto-N-tetraose, lactodifucotetraose, lacto-N-tetraose, lacto-N-neotetraose, lacto-N-fucopentaose II, lacto-N-fucopentaose I, lacto-N-fucopentaose Ill, lacto-N-fucopentaose V, lacto-N-fucopentaose VI, sialyllacto-N-tetraose c, sialyllacto-N-tetraose b, sialyllacto-N-tetraose a, lacto-N-difucohexaose I, lacto-N-difucohexaose II, lacto-N-hexaose, lacto-N-neohexaose, para-lacto-N-hexaose, monofucosylmonosialyllacto-N-tetraose c, monofucosyl para-lacto-N-hexaose, monofucosyllacto-N-hexaose Ill, isomeric fucosylated lacto-N-hexaose Ill, isomeric fucosylated lacto-N-hexaose I, sialyllacto-N-hexaose, sialyllacto-N-neohexaose II, difucosyl-para-lacto-N-hexaose, difucosyllacto-N-hexaose, difucosyllacto-N-hexaose a, difucosyllacto-N-hexaose c, galactosylated chitosan, fucosylated oligosaccharides, neutral oligosaccharide and/or sialylated oligosaccharides.

Human milk oligosaccharides are also known as human identical milk oligosaccharides which are chemically identical to the human milk oligosaccharides found in human breast milk, but which are biotechnologically produced (e.g. using cell free systems or cells and organisms comprising a bacterium, a fungus, a yeast, a plant, animal, or protozoan cell, preferably metabolically engineered cells and organisms). Human identical milk oligosaccharides are marketed under the name HiMO. HMOs comprise fucosylated oligosaccharides, non-fucosylated neutral oligosaccharides and sialylated oligosaccharides (see e.g. Chen X., Chapter Four: Human Milk Oligosaccharides (HMOS): Structure, Function, and Enzyme-Catalyzed Synthesis in Adv. Carbohydr. Chem. Biochem. 72, 113 (2015)). Examples of HMOs comprise 3-fucosyllactose, 2′-fucosyllactose, 2′,3-difucosyllactose, 6′-sialyllactose, 3′-sialyllactose, LN3, lacto-N-tetraose, lacto-N-neotetraose, lacto-N-fucopentaose II, lacto-N-fucopentaose I, lacto-N-fucopentaose Ill, lacto-N-fucopentaose V, lacto-N-fucopentaose VI, sialyllacto-N-tetraose c, sialyllacto-N-tetraose b, sialyllacto-N-tetraose a, difucosyllacto-N-tetraose, lacto-N-hexaose, lacto-N-difucohexaose I, lacto-N-difucohexaose II, disialyllacto-N-tetraose, fucosyllacto-N-hexaose, difucosyllacto-N-hexaose, fucodisialyllacto-N-hexaose, disialyllacto-N-hexaose.

The term “amino-sugar” as used herein refers to a sugar molecule in which a hydroxyl group has been replaced with an amine group. As used herein, an antigen of the human ABO blood group system is an oligosaccharide. Such antigens of the human ABO blood group system are not restricted to human structures. Said structures involve the A determinant GalNAc-alpha1,3(Fuc-alpha1,2)-Gal-, the B determinant Gal-alpha1,3(Fuc-alpha1,2)-Gal- and the H determinant Fuc-alpha1,2-Gal- that are present on disaccharide core structures comprising Gal-beta1,3-GlcNAc, Gal-beta1,4-GlcNAc, Gal-beta1,3-GalNAc and Gal-beta1,4-Glc.

The terms “LNT II”, “LNT-II”, “LN3”, “lacto-N-triose II”, “lacto-N-triose II”, “lacto-N-triose”, “lacto-N-triose” or “GlcNAc(31-3Galβ1-4Glc” as used in the present invention, are used interchangeably.

The terms “LNT”, “lacto-N-tetraose”, “lacto-N-tetraose” or “Gal 31-3GlcNAc(31-3Galβ1-4Glc” as used in the present invention, are used interchangeably.

The terms “LNnT”, “lacto-N-neotetraose”, “lacto-N-neotetraose”, “neo-LNT” or “Galβ1-4GlcNAcβ1-3Galβ1-4Glc” as used in the present invention, are used interchangeably.

The terms “LSTa”, “LS-Tetrasaccharide a”, “Sialyl-lacto-N-tetraose a”, “sialyllacto-N-tetraose a” or “Neu5Ac-a2,3-Gal-b1,3-GlcNAc-b1,3-Gal-b1,4-Glc” as used in the present invention, are used interchangeably.

The terms “LSTb”, “LS-Tetrasaccharide b”, “Sialyl-lacto-N-tetraose b”, “sialyllacto-N-tetraose b” or “Gal-b1,3-(Neu5Ac-a2,6)-GlcNAc-b1,3-Gal-b1,4-Glc” as used in the present invention, are used interchangeably.

The terms “LSTc”, “LS-Tetrasaccharide c”, “Sialyl-lacto-N-tetraose c”, “sialyllacto-N-tetraose c”, “sialyllacto-N-neotetraose c” or “Neu5Ac-a2,6-Gal-b1,4-GlcNAc-b1,3-Gal-b1,4-Glc” as used in the present invention, are used interchangeably.

The terms “LSTd”, “LS-Tetrasaccharide d”, “Sialyl-lacto-N-tetraose d”, “sialyllacto-N-tetraose d”, “sialyllacto-N-neotetraose d” or “Neu5Ac-a2,3-Gal-b1,4-GlcNAc-b1,3-Gal-b1,4-Glc” as used in the present invention, are used interchangeably.

The term “membrane transporter proteins” as used herein refers to proteins that are part of or interact with the cell membrane and control the flow of molecules and information across the cell. The membrane proteins are thus involved in transport, be it import into or export out of the cell. Such membrane transporter proteins can be but are not limited to porters, P-P-bond-hydrolysis-driven transporters, β-Barrel Porins, auxiliary transport proteins and phosphotransfer-driven group translocators (Forrest et al., Biochim. Biophys. Acta 1807 (2011) 167-188; Lengeler, J. Mol. Microbiol. Biotechnol. 25 (2015) 79-93; Moraes and Reithmeier, Biochim. Biophys. Acta 1818 (2012), 2687-2706; Saier et al., Nucleic Acids Res. 44 (2016) D372-D379).

The major facilitator superfamily (MFS) is a superfamily of membrane transporter proteins that are single-polypeptide secondary carriers with InterPro domain IPR036259, capable of transporting small solutes in response to chemiosmotic ion gradients (Pao et al., J. Microbiol. Mol. Biol. Rev. 62 (1998) 1-34; Walmsley et al., Trends Biochem. Sci. 23 (1998) 476-481; Wang et al., Jr. Biochim. Biophys. Acta Biomembr. 1862 (2020) 183277; Teelucksingh et al. 202 (2020) e00367-20).

“SET” or “Sugar Efflux Transporter” as used herein is part of the MFS superfamily and refers to membrane proteins of the SET family which are proteins with InterPRO domain IPR004750 and/or are proteins that belong to the eggNOGv5.0.0 family ENOG410XTE9. This family of proteins is an efflux system for lactose, glucose, aromatic glucosides and galactosides, cellobiose, maltose, a-methyl glucoside and other sugar compounds. They are found in both Gram-negative and Gram-positive bacteria (Liu et al., Mol. Microbiol. 31 (1999) 1845-1851; Liu et al., J. Biol. Chem. 274 (1999) 22977-22984; Sun and Vanderpool, J. Bacteriol. 193 (2011) 143-153).

The term “Siderophore” as used herein is referring to the secondary metabolite of various microorganisms which are mainly ferric ion specific chelators (Neilands, J. Biol. Chem. 270 (1995) 26723-26726). A transporter is needed to export the siderophore outside the cell. Four superfamilies of membrane proteins are identified so far in this process: the major facilitator superfamily (MFS); the Multidrug/Oligosaccharidyl-lipid/Polysaccharide Flippase Superfamily (MOP); the resistance, nodulation and cell division superfamily (RND); and the ABC superfamily (Teelucksingh et al. 202 (2020) e00367-20). In general, the genes involved in siderophore export are clustered together with the siderophore biosynthesis genes. The term “siderophore exporter” as used herein refers to such transporters needed to export the siderophore outside of the cell.

The ATP-binding cassette (ABC) superfamily contains both uptake and efflux transport systems, and the members of these two groups generally cluster loosely together. ATP hydrolysis without protein phosphorylation energizes transport. There are dozens of families within the ABC superfamily, and family generally correlates with substrate specificity (Davidson et al. Microbiol. Mol. Biol. Rev. 72 (2008) 317-364; Goffeau et al. (2013). “ABC Transporters”. In Lane W J, Lennarz MD (eds.). Encyclopedia of Biological Chemistry (Second ed.). London: Academic Press. pp. 7-11).

The terms “cell genetically modified for the production of a sialylated di- and/or oligosaccharide” or “cell metabolically engineered for the production of a sialylated di- and/or oligosaccharide” within the context of the present disclosure refers to a cell of a microorganism which is genetically manipulated to comprise at least one sialyltransferase combined with any one or more of i) a gene encoding a glycosyltransferase necessary for the synthesis of said sialylated di- and/or oligosaccharide, ii) a biosynthetic pathway to produce a nucleotide donor suitable to be transferred by said glycosyltransferase to a carbohydrate precursor, and/or iii) a biosynthetic pathway to produce a precursor or a mechanism of internalization of a precursor from the culture medium into the cell where it is glycosylated to produce the sialylated di- and/or oligosaccharide.

The term “pathway for production of a sialylated di- and/or oligosaccharide” as used herein is a biochemical pathway consisting of the enzymes and their respective genes involved in the synthesis of a sialylated di- and/or oligosaccharide as defined herein. Said pathway for production of a sialylated di- and/or oligosaccharide can comprise but is not limited to pathways involved in the synthesis of a nucleotide-activated sugar and the transfer of said nucleotide-activated sugar to an acceptor to create a sialylated di- and/or oligosaccharide of the present invention. An example of such pathway is a sialylation pathway. Further examples of such pathway comprise but are not limited to a fucosylation, galactosylation, N-acetylglucosaminylation, N-acetylgalactosaminylation, mannosylation, N-acetylmannosaminylation pathway.

A ‘sialylation pathway’ is a biochemical pathway consisting of at least one of the enzymes and their respective genes chosen from the list comprising an L-glutamine-D-fructose-6-phosphate aminotransferase, a phosphoglucosamine mutase, an N-acetylglucosamine-6-P deacetylase, an N-acylglucosamine 2-epimerase, a UDP-N-acetylglucosamine 2-epimerase, an N-acetylmannosamine-6-phosphate 2-epimerase, a UDP-GlcNAc 2-epimerase/kinase, a glucosamine 6-phosphate N-acetyltransferase, an N-acetylglucosamine-6-phosphate phosphatase, a phosphoacetylglucosamine mutase, an N-acetylglucosamine 1-phosphate uridylyltransferase, a glucosamine-1-phosphate acetyltransferase, an Neu5Ac synthase, an N-acetylneuraminate lyase, an N-acylneuraminate-9-phosphate synthase, an N-acylneuraminate-9-phosphatase, a sialic acid transporter and a CMP-sialic acid synthase, combined with a sialyltransferase leading to α 2,3; α 2,6 and/or α 2,8 sialylated oligosaccharides. The terms “L-glutamine-D-fructose-6-phosphate aminotransferase”, “glutamine---fructose-6-phosphate transaminase (isomerizing)”, “hexosephosphate aminotransferase”, “glucosamine-6-phosphate isomerase (glutamine-forming)”, “glutamine-fructose-6-phosphate transaminase (isomerizing)”, “D-fructose-6-phosphate amidotransferase”, “fructose-6-phosphate aminotransferase”, “glucosaminephosphate isomerase”, “glucosamine 6-phosphate synthase”, “GlcN6P synthase”, “GFA”, “glms”, “glmS” and “glmS*54” are used interchangeably and refer to an enzyme that catalyses the conversion of D-fructose-6-phosphate into D-glucosamine-6-phosphate using L-glutamine.

The terms “glucosamine-6-P deaminase”, “glucosamine-6-phosphate deaminase”, “GlcN6P deaminase”, “glucosamine-6-phosphate isomerase”, “glmD” and “nagB” are used interchangeably and refer to an enzyme that catalyses the reversible isomerization-deamination of glucosamine-6-phosphate (GlcN6P) to form fructose-6-phosphate and an ammonium ion.

The terms “phosphoglucosamine mutase” and “glmM” are used interchangeably and refer to an enzyme that catalyses the conversion of glucosamine-6-phosphate to glucosamine-1-phosphate. Phosphoglucosamine mutase can also catalyse the formation of glucose-6-P from glucose-1-P, although at a 1400-fold lower rate.

The terms “N-acetylglucosamine-6-P deacetylase”, “N-acetylglucosamine-6-phosphate deacetylase” and “nagA” are used interchangeably and refer to an enzyme that catalyses the hydrolysis of the N-acetyl group of N-acetylglucosamine-6-phosphate (GlcNAc-6-P) to yield glucosamine-6-phosphate (GlcN6P) and acetate.

An N-acylglucosamine 2-epimerase is an enzyme that catalyses the reaction N-acyl-D-glucosamine=N-acyl-D-mannosamine. Alternative names for this enzyme comprise N-acetylglucosamine 2-epimerase, N-acetyl-D-glucosamine 2-epimerase, GlcNAc 2-epimerase, N-acyl-D-glucosamine 2-epimerase and N-acetylglucosamine epimerase.

A UDP-N-acetylglucosamine 2-epimerase is an enzyme that catalyses the reaction N-acetyl-D-glucosamine=N-acetylmannosamine. Alternative names for this enzyme comprise UDP-N-acylglucosamine 2-epimerase, UDP-GlcNAc-2-epimerase, “neuC” and UDP-N-acetyl-D-glucosamine 2-epimerase.

An N-acetylmannosamine-6-phosphate 2-epimerase is an enzyme that catalyses the reaction N-acetyl-D-glucosamine 6-phosphate=N-acetyl-D-mannosamine 6-phosphate.

A bifunctional UDP-GlcNAc 2-epimerase/kinase is a bifunctional enzyme that catalyses the reaction UDP-N-acetyl-D-glucosamine=N-acetyl-D-mannosamine and the reaction N-acetyl-D-mannosamine+ATP=ADP+N-acetyl-D-mannosamine 6-phosphate.

A glucosamine 6-phosphate N-acetyltransferase is an enzyme that catalyses the transfer of an acetyl group from acetyl-CoA to D-glucosamine-6-phosphate thereby generating a free CoA and N-acetyl-D-glucosamine 6-phosphate. Alternative names comprise aminodeoxyglucosephosphate acetyltransferase, D-glucosamine-6-P N-acetyltransferase, glucosamine 6-phosphate acetylase, glucosamine 6-phosphate N-acetyltransferase, glucosamine-phosphate N-acetyltransferase, glucosamine-6-phosphate acetylase, N-acetylglucosamine-6-phosphate synthase, phosphoglucosamine acetylase, phosphoglucosamine N-acetylase phosphoglucosamine N-acetylase, phosphoglucosamine transacetylase, GNA and GNA1.

The term “N-acetylglucosamine-6-phosphate phosphatase” refers to an enzyme that dephosphorylates N-acetylglucosamine-6-phosphate (GlcNAc-6-P) hereby synthesizing N-acetylglucosamine (GlcNAc).

The term “N-acetylmannosamine-6-phosphate phosphatase” refers to an enzyme that dephosphorylates N-acetylmannosamine-6-phosphate (ManNAc-6P) to N-acetylmannosamine (ManNAc).

The terms “N-acetylneuraminate kinase”, “ManNAc kinase”, “N-acetyl-D-mannosamine kinase” and “nanK” are used interchangeably and refer to an enzyme that phosphorylates ManNAc to synthesize N-acetylmannosamine-phosphate (ManNAc-6-P).

The terms “N-acetylmannosamine-6-phosphate 2-epimerase”, “ManNAc-6-P isomerase”, “ManNAc-6-P 2-epimerase”, N-acetylglucosamine-6P 2-epimerase and “nanE” are used interchangeably and refer to an enzyme that converts ManNAc-6-P to N-acetylglucosamine-6-phosphate (GlcNAc-6-P).

The terms “phosphoacetylglucosamine mutase”, “acetylglucosamine phosphomutase”, “acetylaminodeoxyglucose phosphomutase”, “phospho-N-acetylglucosamine mutase” and “N-acetyl-D-glucosamine 1,6-phosphomutase” are used interchangeably and refer to an enzyme that catalyses the conversion of N-acetyl-glucosamine 1-phosphate into N-acetylglucosamine 6-phosphate.

The terms “N-acetylglucosamine 1-phosphate uridylyltransferase”, “N-acetylglucosamine-1-phosphate uridyltransferase”, “UDP-N-acetylglucosamine diphosphorylase”, “UDP-N-acetylglucosamine pyrophosphorylase”, “uridine diphosphoacetylglucosamine pyrophosphorylase”, “UTP:2-acetamido-2-deoxy-alpha-D-glucose-1-phosphate uridylyltransferase”, “UDP-GlcNAc pyrophosphorylase”, “GlmU uridylyltransferase”, “Acetylglucosamine 1-phosphate uridylyltransferase”, “UDP-acetylglucosamine pyrophosphorylase”, “uridine diphosphate-N-acetylglucosamine pyrophosphorylase”, “uridine diphosphoacetylglucosamine phosphorylase”, and “acetylglucosamine 1-phosphate uridylyltransferase” are used interchangeably and refer to an enzyme that catalyses the conversion of N-acetylglucosamine 1-phosphate (GlcNAc-1-P) into UDP-N-acetylglucosamine (UDP-GlcNAc) by the transfer of uridine 5-monophosphate (from uridine 5-triphosphate (UTP)).

The term glucosamine-1-phosphate acetyltransferase refers to an enzyme that catalyses the transfer of the acetyl group from acetyl coenzyme A to glucosamine-1-phosphate (GlcN-1-P) to produce N-acetylglucosamine-1-phosphate (GlcNAc-1-P).

The term “glmU” refers to a bifunctional enzyme that has both N-acetylglucosamine-1-phosphate uridyltransferase and glucosamine-1-phosphate acetyltransferase activity and that catalyses two sequential reactions in the de novo biosynthetic pathway for UDP-GlcNAc. The C-terminal domain catalyses the transfer of acetyl group from acetyl coenzyme A to GlcN-1-P to produce GlcNAc-1-P, which is converted into UDP-GlcNAc by the transfer of uridine 5-monophosphate, a reaction catalysed by the N-terminal domain.

The terms “Neu5Ac synthase”, “N-acetylneuraminic acid synthase”, “N-acetylneuraminate synthase”, “sialic acid synthase”, “NeuAc synthase”, “NeuB”, “NeuB1”, “NANA condensing enzyme”, “N-acetylneuraminate lyase synthase”, “N-acetylneuraminic acid condensing enzyme” as used herein are used interchangeably and refer to an enzyme capable to synthesize sialic acid (Neu5Ac) from N-acetylmannosamine (ManNAc) in a reaction using phosphoenolpyruvate (PEP).

The terms “N-acetylneuraminate lyase”, “Neu5Ac lyase”, “N-acetylneuraminate pyruvate-lyase”, “N-acetylneuraminic acid aldolase”, “NALase”, “sialate lyase”, “sialic acid aldolase”, “sialic acid lyase” and “nanA” are used interchangeably and refer to an enzyme that degrades N-acetylneuraminate into N-acetylmannosamine (ManNAc) and pyruvate.

The terms “N-acylneuraminate-9-phosphate synthase”, “N-acylneuraminate-9-phosphate synthetase”, “NANA synthase”, “NANAS”, “NANS”, “NmeNANAS”, “N-acetylneuraminate pyruvate-lyase (pyruvate-phosphorylating)” as used herein are used interchangeably and refer to an enzyme capable to synthesize N-acylneuraminate-9-phosphate from N-acetylmannosamine-6-phosphate (ManNAc-6-phosphate) in a reaction using phosphoenolpyruvate (PEP).

The term “N-acylneuraminate-9-phosphatase” refers to an enzyme capable to dephosphorylate N-acylneuraminate-9-phosphate to synthesise N-acylneuraminate.

The terms “CMP-sialic acid synthase”, “N-acylneuraminate cytidylyltransferase”, “CMP-sialate synthase”, “CMP-NeuAc synthase”, “NeuA” and “CMP-N-acetylneuraminic acid synthase” as used herein are used interchangeably and refer to an enzyme capable to synthesize CMP-N-acetylneuraminate from N-acetylneuraminate using CTP in the reaction.

A ‘fucosylation pathway’ as used herein is a biochemical pathway comprising at least one of the enzymes and their respective genes chosen from the list comprising mannose-6-phosphate isomerase, phosphomannomutase, mannose-1-phosphate guanylyltransferase, GDP-mannose 4,6-dehydratase, GDP-L-fucose synthase, fucose permease, fucose kinase, fucose-1-phosphate guanylyltransferase combined with a fucosyltransferase leading to a 1,2; a 1,3; a 1,4 and/or a 1,6 fucosylated compounds.

A ‘galactosylation pathway’ as used herein is a biochemical pathway comprising at least one of the enzymes and their respective genes chosen from the list comprising galactose-1-epimerase, galactokinase, glucokinase, galactose-1-phosphate uridylyltransferase, UDP-glucose 4-epimerase, glucose-1-phosphate uridylyltransferase, phosphoglucomutase combined with a galactosyltransferase leading to a galactosylated compound comprising a mono-, di-, or oligosaccharide having an alpha or beta bound galactose on any one or more of the 2, 3, 4 and 6 hydroxyl group of said mono-, di-, or oligosaccharide.

An ‘N-acetylglucosaminylation pathway’ as used herein is a biochemical pathway comprising at least one of the enzymes and their respective genes chosen from the list comprising L-glutamine-D-fructose-6-phosphate aminotransferase, N-acetylglucosamine-6-phosphate deacetylase, phosphoglucosamine mutase, N-acetylglucosamine-1-phosphate uridylyltransferase/glucosamine-1-phosphate acetyltransferase combined with a glycosyltransferase leading to a GlcNAc-modified compound comprising a mono-, di-, or oligosaccharide having an alpha or beta bound N-acetylglucosamine (GlcNAc) on any one or more of the 3, 4 and 6 hydroxyl group of said mono-, di- or oligosaccharide.

An ‘N-acetylgalactosaminylation pathway’ as used herein is a biochemical pathway comprising at least one of the enzymes and their respective genes chosen from the list comprising L-glutamine-D-fructose-6-phosphate aminotransferase, phosphoglucosamine mutase, N-acetylglucosamine 1-phosphate uridylyltransferase, glucosamine-1-phosphate acetyltransferase, UDP-N-acetylglucosamine 4-epimerase, UDP-glucose 4-epimerase, N-acetylgalactosamine kinase and UDP-GalNAc pyrophosphorylase combined with a glycosyltransferase leading to a GalNAc-modified compound comprising a mono-, di- or oligosaccharide having an alpha or beta bound N-acetylgalactosamine on said mono-, di- or oligosaccharide.

A ‘mannosylation pathway’ as used herein is a biochemical pathway comprising at least one of the enzymes and their respective genes chosen from the list comprising mannose-6-phosphate isomerase, phosphomannomutase and mannose-1-phosphate guanylyltransferase combined with a glycosyltransferase leading to a mannosylated compound comprising a mono-, di- or oligosaccharide having an alpha or beta bound mannose on said mono-, di- or oligosaccharide.

An ‘N-acetylmannosaminylation pathway’ as used herein is a biochemical pathway comprising at least one of the enzymes and their respective genes chosen from the list comprising L-glutamine-D-fructose-6-phosphate aminotransferase, glucosamine-6-phosphate deaminase, phosphoglucosamine mutase, N-acetylglucosamine-6-phosphate deacetylase, glucosamine 6-phosphate N-acetyltransferase, N-acetylglucosamine-1-phosphate uridyltransferase, glucosamine-1-phosphate acetyltransferase, UDP-GlcNAc 2-epimerase and ManNAc kinase combined with a glycosyltransferase leading to a ManNAc-modified compound comprising a mono-, di- or oligosaccharide having an alpha or beta bound N-acetylmannosamine on said mono-, di- or oligosaccharide.

The terms “pyruvate dehydrogenase”, “pyruvate oxidase”, “POX”, “poxB” and “pyruvate:ubiquinone-8 oxidoreductase” are used interchangeably and refer to an enzyme that catalyses the oxidative decarboxylation of pyruvate to produce acetate and C02.

The terms “lactate dehydrogenase”, “D-lactate dehydrogenase”, “IdhA”, “hslI”, “htpH”, “D-LDH”, “fermentative lactate dehydrogenase” and “D-specific 2-hydroxyacid dehydrogenase” are used interchangeably and refer to an enzyme that catalyses the conversion of lactate into pyruvate hereby generating NADH.

The term “purified” refers to material that is substantially or essentially free from components that interfere with the activity of the biological molecule. For cells, saccharides, nucleic acids, and polypeptides, the term “purified” refers to material that is substantially or essentially free from components that normally accompany the material as found in its native state. Typically, purified saccharides, oligosaccharides, proteins or nucleic acids of the invention are at least about 50%, 55%, 60%, 65%, 70%, 75%, 80% or 85% pure, usually at least about 90%, 91%, 92%, 93%, 94%, 95%, 96%, 97%, 98%, or 99.0% pure as measured by band intensity on a silver-stained gel or other method for determining purity. Purity or homogeneity can be indicated by a number of means well known in the art, such as polyacrylamide gel electrophoresis of a protein or nucleic acid sample, followed by visualization upon staining. For certain purposes high resolution will be needed and HPLC or a similar means for purification utilized. For di- and/or oligosaccharides, purity can be determined using methods such as but not limited to thin layer chromatography, gas chromatography, NMR, HPLC, capillary electrophoresis or mass spectroscopy. Further herein, the terms “contaminants” and “impurities” preferably mean particulates, cells, cell components, metabolites, cell debris, proteins, peptides, amino acids, nucleic acids, glycolipids and/or endotoxins which can be present in an aqueous medium like e.g. a cultivation or an incubation.

The term “clarifying” as used herein refers to the act of treating an aqueous medium like e.g. a cultivation or an incubation, to remove suspended particulates and contaminants from the production process, like e.g. cells, cell components, insoluble metabolites and debris, that could interfere with the eventual purification of the one or more bioproduct(s). Such treatment can be carried out in a conventional manner by centrifugation, flocculation, flocculation with optional ultrasonic treatment, gravity filtration, microfiltration, foam separation or vacuum filtration (e.g. through a ceramic filter which can include a Celite™ filter aid).

The term “cultivation” refers to the culture medium wherein the cell is cultivated, or fermented, the cell itself, and a sialylated oligosaccharide, preferably a 6′sialylated oligosaccharide, more preferably LSTc, that is produced by the cell in whole broth, i.e. inside (intracellularly) as well as outside (extracellularly) of the cell.

The term “incubation” refers to a mixture wherein a sialylated oligosaccharide, preferably a 6′sialylated oligosaccharide, more preferably LSTc, is produced. Said mixture can comprise one or more enzyme(s), one or more precursor(s) and one or more acceptor(s) as defined herein present in a buffered solution and incubated for a certain time at a certain temperature enabling production of a sialylated oligosaccharide, preferably a 6′sialylated oligosaccharide, more preferably LSTc, catalysed by said one or more enzyme(s) using said one or more precursor(s) and said one or more acceptor(s) in said mixture. Said mixture can also comprise i) the cell obtained after cultivation or incubation, optionally said cell is subjected to cell lysis, ii) a buffered solution or the cultivation or incubation medium wherein the cell was cultivated or fermented, and iii) a sialylated oligosaccharide, preferably a 6′sialylated oligosaccharide, more preferably LSTc, that is produced by the cell in whole broth, i.e. inside (intracellularly) as well as outside (extracellularly) of the cell. Said incubation can also be the cultivation as defined herein.

The terms “reactor” and “incubator” refer to the recipient filled with the cultivation or incubation. Examples of reactors and incubators comprise but are not limited to microfluidic devices, well plates, tubes, shake flasks, fermenters, bioreactors, process vessels, cell culture incubators, CO2 incubators.

As used herein, the term “cell productivity index (CPI)” refers to the mass of the sialylated oligosaccharide produced by the cells divided by the mass of the cells produced in the culture or cultivation.

The term “precursor” as used herein refers to substances that are taken up or synthetized by the cell for the specific production of a sialylated oligosaccharide according to the present invention. In this sense a precursor can be an acceptor as defined herein, but can also be another substance, metabolite, that is first modified within the cell as part of the biochemical synthesis route of a sialylated oligosaccharide, preferably a 6′sialylated oligosaccharide, more preferably LSTc. The term “precursor” as used herein is also to be understood as a chemical compound that participates in a chemical or enzymatic reaction to produce another compound like e.g. an intermediate or an acceptor as defined herein, as part in the metabolic pathway of a sialylated oligosaccharide, preferably a 6′sialylated oligosaccharide, more preferably LSTc. The term “precursor” as used herein is also to be understood as a donor that is used by a glycosyltransferase to modify an acceptor as defined herein with a sugar moiety in a glycosidic bond, as part in the metabolic pathway of a sialylated oligosaccharide, preferably a 6′sialylated oligosaccharide, more preferably LSTc.

Examples of such precursors comprise the acceptors as defined herein, and/or dihydroxyacetone, glucosamine, N-acetylglucosamine, N-acetylmannosamine, galactosamine, N-acetylgalactosamine, galactosyllactose, phosphorylated sugars or sugar phosphates like e.g. but not limited to glucose-1-phosphate, galactose-1-phosphate, glucose-6-phosphate, fructose-6-phosphate, fructose-1,6-bisphosphate, mannose-6-phosphate, mannose-1-phosphate, glycerol-3-phosphate, glyceraldehyde-3-phosphate, dihydroxyacetone-phosphate, glucosamine-6-phosphate, N-acetylglucosamine-6-phosphate, N-acetylmannosamine-6-phosphate, N-acetylglucosamine-1-phosphate, N-acetylneuraminic acid-9-phosphate and nucleotide-activated sugars like nucleotide diphospho-sugars and nucleotide monophospho-sugars as defined herein like e.g. UDP-glucose, UDP-galactose, UDP-N-acetylglucosamine, CMP-sialic acid, GDP-mannose, GDP-4-dehydro-6-deoxy-α-D-mannose, GDP-fucose.

Optionally, the cell is transformed to comprise and to express at least one nucleic acid sequence encoding a protein selected from the group consisting of lactose transporter, N-acetylneuraminic acid transporter, fucose transporter, glucose transporter, galactose transporter, transporter for a nucleotide-activated sugar wherein said transporter internalizes a to the medium added precursor for the synthesis of the sialylated oligosaccharide, preferably 6′sialylated oligosaccharide, more preferably LSTc, of present invention.

The term “acceptor” as used herein refers to a mono-, di- or oligosaccharide, which can be modified by a glycosyltransferase. Examples of such acceptors comprise glucose, galactose, fructose, glycerol, sialic acid, fucose, mannose, maltose, sucrose, lactose, lacto-N-triose, lacto-N-tetraose (LNT), lacto-N-neotetraose (LNnT), lacto-N-pentaose (LNP), lacto-N-neopentaose, para lacto-N-pentaose, para lacto-N-neopentaose, lacto-N-novopentaose I, lacto-N-hexaose (LNH), lacto-N-neohexaose (LNnH), para lacto-N-neohexaose (pLNnH), para lacto-N-hexaose (pLNH), lacto-N-heptaose, lacto-N-neoheptaose, para lacto-N-neoheptaose, para lacto-N-heptaose, lacto-N-octaose (LNO), lacto-N-neooctaose, iso lacto-N-octaose, para lacto-N-octaose, iso lacto-N-neooctaose, novo lacto-N-neooctaose, para lacto-N-neooctaose, iso lacto-N-nonaose, novo lacto-N-nonaose, lacto-N-nonaose, lacto-N-decaose, iso lacto-N-decaose, novo lacto-N-decaose, lacto-N-neodecaose, and oligosaccharide containing 1 or more N-acetyllactosamine units and/or 1 or more lacto-N-biose units or an intermediate into oligosaccharide, fucosylated and sialylated versions thereof, ceramide, N-acylated sphingoid, glucosylceramide, lactosylceramide, sphingosine, phytosphingosine, sphingosine synthons, peptide backbones with beta-GlcNAc-Asn residues, glycoproteins with terminal GlcNAc and Gal residues, immunoglobulins.

### DETAILED DESCRIPTION OF THE INVENTION

According to a first aspect, the present invention provides an alpha-2,6-sialyltransferase having alpha-2,6-sialyltransferase activity on the terminal galactose (Gal) residue of lacto-N-neotetraose (LNnT, Gal-β1,4-GlcNAc-β1,3-Gal-β1,4-Glc) and comprising an amino acid sequence that is at least 60.0% identical over a stretch of at least 100 amino acid residues, preferably at least 150 amino acid residues, more preferably at least 200 amino acid residues, to the amino acid sequence as represented by SEQ ID NO 1.

Preferably, said alpha-2,6-sialyltransferase comprises an amino acid sequence that is at least 60.0%, at least 65.0%, at least 70.0%, at least 75.0%, at least 80.0%, at least 85.0%, at least 90.0%, at least 95.0%, at least 96.0%, at least 97.0%, at least 98.0%, at least 98.5%, or at least 99.0% identical to the amino acid sequence as represented by SEQ ID NO 1 over a stretch of at least 100, at least 110, at least 120, at least 130, at least 140, at least 150, at least 160, at least 170, at least 180, at least 190, at least 200, at least 210, at least 220, at least 230, at least 240, at least 250, at least 260, at least 270, at least 280, at least 290 or at least 300 amino acid residues.

More preferably, said alpha-2,6-sialyltransferase comprises an amino acid sequence that is at least 60.0%, at least 65.0%, at least 70.0%, at least 75.0%, at least 80.0%, at least 85.0%, at least 90.0%, at least 95.0%, at least 96.0%, at least 97.0%, at least 98.0%, at least 98.5%, or at least 99.0% identical to the full-length amino acid sequence as represented by SEQ ID NO 1.

Alternatively, said alpha-2,6-sialyltransferase comprises an amino acid sequence comprising a fragment of an amino acid sequence as represented by SEQ ID NO 1 and having alpha-2,6-sialyltransferase activity on the terminal Gal residue of LNnT.

Most preferably, said alpha-2,6-sialyltransferase comprises an amino acid sequence as represented by SEQ ID NO 1.

In a second aspect, the present invention provides a method for the production of a sialylated oligosaccharide, preferably a 6′sialylated oligosaccharide, more preferably LSTc (Neu5Ac-α2,6-Gal-β1,4-GlcNAc-β1,3-Gal-β1,4-Glc). The method comprising contacting an alpha-2,6-sialyltransferase as described herein with a mixture comprising a donor comprising a sialic acid residue, and an acceptor chosen from the list comprising an oligosaccharide or disaccharide, under conditions where said alpha-2,6-sialyltransferase catalyses the transfer of a sialic acid residue from the donor to the acceptor, thereby producing said sialylated oligosaccharide. Preferably, the sialylated oligosaccharide is a 6′sialylated oligosaccharide, more preferably LSTc and said acceptor substrate is lacto-N-neotetraose (LNnT, Gal-β1,4-GlcNAc-β1,3-Gal-β1,4-Glc).

In a specific embodiment, the present invention provides a method for the production of a sialylated oligosaccharide, preferably a 6′sialylated oligosaccharide, more preferably LSTc (Neu5Ac-α2,6-Gal-β1,4-GlcNAc-β1,3-Gal-β1,4-Glc), wherein said method comprises the steps of:
a) providing
   i. CMP-sialic acid
   ii. an acceptor, preferably lacto-N-neotetraose (LNnT, Gal-β1,4-GlcNAc-β1,3-Gal-β1,4-Glc)
   iii. a sialyltransferase, wherein said sialyltransferase is an alpha-2,6-sialyltransferase which has alpha-2,6-sialyltransferase activity on the terminal galactose (Gal) residue of lacto-N-neotetraose (LNnT, Gal-β1,4-GlcNAc-β1,3-Gal-β1,4-Glc) and comprises an amino acid sequence that is at least 60.0% identical over a stretch of at least 100 amino acid residues, preferably at least 150 amino acid residues, more preferably at least 200 amino acid residues, to the amino acid sequence as represented by SEQ ID NO 1,
b) contacting said sialyltransferase and CMP-sialic acid with said acceptor, preferably LNnT, under conditions where the sialyltransferase catalyses the transfer of a sialic acid residue from said CMP-sialic acid to the acceptor resulting in the production of a sialylated oligosaccharide, preferably a 6′sialylated oligosaccharide, more preferably LSTc (Neu5Ac-α2,6-Gal-β1,4-GlcNAc-β1,3-Gal-β1,4-Glc),
c) preferably, separating said produced sialylated oligosaccharide.

Preferably, the sialyltransferase is any one of the alpha-2,6-sialyltransferase as described herein.

In an alternative specific embodiment, the present invention provides a method wherein a cell extract comprising an alpha-2,6-sialyltransferase as described herein is contacted with a mixture comprising a donor comprising a sialic acid residue, and an acceptor comprising an oligosaccharide or disaccharide, under conditions where said alpha-2,6-sialyltransferase catalyzes the transfer of a sialic acid residue from the donor to the acceptor, thereby producing said sialylated oligosaccharide. Preferably said sialylated oligosaccharide is a 6′sialylated oligosaccharide, more preferably LSTc.

In an alternative embodiment of the method of the present invention, the sialylated oligosaccharide is produced in a cell-free system. Preferably said sialylated oligosaccharide is a 6′sialylated oligosaccharide, more preferably LSTc.

In another aspect, the present invention provides a method for the production of a sialylated oligosaccharide, preferably a 6′sialylated oligosaccharide, more preferably LSTc (Neu5Ac-α2,6-Gal-β1,4-GlcNAc-β1,3-Gal-β1,4-Glc), wherein said method comprises the steps of:
i. providing a cell, preferably a single cell, expressing, preferably heterologously expressing, more preferably overexpressing, even more preferably heterologously overexpressing, an alpha-2,6-sialyltransferase as described herein,
ii. providing CMP-sialic acid, optionally said CMP-sialic acid is produced by said cell, and
iii. providing an oligosaccharide or disaccharide, optionally said oligosaccharide or disaccharide is produced by said cell, and
iv. cultivating and/or incubating said cell under conditions permissive to express said alpha-2,6-sialyltransferase, optionally permissive to produce said CMP-sialic acid and/or said oligosaccharide or disaccharide,

preferably, separating said sialylated oligosaccharide, preferably 6′sialylated oligosaccharide, more preferably LSTc, from said cultivation or incubation.

In a specific embodiment of the method of the present invention, the sialylated oligosaccharide is produced by a cell, preferably a single cell, wherein said cell expresses i) an alpha-2,6-sialyltransferase which has alpha-2,6-sialyltransferase activity on the terminal Gal residue of LNnT and comprises an amino acid sequence that is at least 60.0% identical over a stretch of at least 100 amino acid residues, preferably at least 150 amino acid residues, more preferably at least 200 amino acid residues, to the amino acid sequence as represented by SEQ ID NO 1 or ii) any of the alpha-2,6-sialyltransferases as described herein. The cell used can be a metabolically engineered cell as described herein, preferably wherein said cell is metabolically engineered for the production of said sialylated oligosaccharide. Preferably said sialylated oligosaccharide is a 6′sialylated oligosaccharide, more preferably LSTc.

In the scope of the present invention, permissive conditions are understood to be conditions relating to physical or chemical parameters including but not limited to temperature, pH, pressure, osmotic pressure and product/precursor/acceptor concentration.

In a particular embodiment, the permissive conditions may include a temperature-range of about 30+/−20 degrees centigrade, a pH-range of 2.0-10.0.

Preferably, the sialylated oligosaccharide is a 6′sialylated oligosaccharide, more preferably LSTc and said acceptor is LNnT.

In an embodiment of the method of present invention, the cultivation medium contains at least one carbon source selected from the group consisting of glucose, fructose, sucrose, and glycerol.

In another embodiment of the method of present invention, the cultivation or incubation medium contains at least one compound selected from the group consisting of lactose, galactose, lacto-N-neotetraose (LNnT), UDP-galactose (UDP-Gal), UDP-N-acetylglucosamine (UDP-GlcNAc), sialic acid and CMP-sialic acid.

Preferably, the sialylated oligosaccharide is recovered from the cultivation or incubation medium and/or the cell or separated from the cultivation or incubation as explained herein.

According to an embodiment of the method of the invention, the conditions permissive to produce said sialylated oligosaccharide comprise the use of a cultivation or incubation medium comprising at least one precursor and/or acceptor for the production of a sialylated oligosaccharide as described herein. Preferably, the cultivation or incubation medium contains at least one precursor and/or acceptor, wherein said precursor is selected from the group comprising a monosaccharide like e.g. galactose, fucose, sialic acid, GlcNAc, GalNAc; a nucleotide-activated sugar like e.g. CMP-sialic acid, UDP-Gal, UDP-GlcNAc, GDP-fucose; a disaccharide like e.g. lactose; and an oligosaccharide like e.g. lacto-N-triose (LN3), lacto-N-tetraose (LNT) and lacto-N-neotetraose (LNnT) and/or wherein said acceptor is selected from the group comprising a disaccharide like e.g. lactose, and an oligosaccharide like e.g. LN3, LNT, LNnT. In a more preferred embodiment of the method of the invention, said precursor is chosen from the list comprising sialic acid, CMP-sialic acid and lactose. In another more preferred embodiment of the method of the invention, said acceptor is LNnT.

According to an alternative and/or additional embodiment of the method of the invention, the conditions permissive to produce said sialylated oligosaccharide comprise adding to the cultivation or incubation medium at least one precursor and/or acceptor feed for the production of said sialylated oligosaccharide. According to an alternative embodiment of the method of the invention, the conditions permissive to produce said sialylated oligosaccharide comprise the use of a cultivation or incubation medium wherein said cultivation or incubation medium lacks any precursor and/or acceptor for the production of said sialylated oligosaccharide and is combined with a further addition to said cultivation or incubation medium of at least one precursor and/or acceptor feed for the production of said sialylated oligosaccharide.

According to an embodiment of the method of the invention, the cultivation or incubation is contained in a reactor or incubator, as defined herein. The volume of said reactor or incubator ranges from microlitre (μL) scale to 10.000 m3 (cubic meter). In a preferred embodiment, the volume of said reactor or incubator ranges from 250 mL (millilitre) to 10.000 m3 (cubic meter).

In a preferred embodiment, the method for the production of a sialylated oligosaccharide as described herein comprises at least one of the following steps:
i) Use of a cultivation or incubation medium comprising at least one precursor and/or acceptor;
ii) Adding to the cultivation or incubation medium in a reactor or incubator at least one precursor and/or acceptor feed wherein the total reactor or incubator volume ranges from 250 mL (millilitre) to 10.000 m³ (cubic meter), preferably in a continuous manner, and preferably so that the final volume of the cultivation or incubation medium is not more than three-fold, preferably not more than two-fold, more preferably less than two-fold of the volume of the cultivation or incubation medium before the addition of said precursor and/or acceptor feed;
iii) Adding to the cultivation or incubation medium in a reactor or incubator at least one precursor and/or acceptor feed wherein the total reactor or incubator volume ranges from 250 mL (millilitre) to 10.000 m³ (cubic meter), preferably in a continuous manner, and preferably so that the final volume of the cultivation or incubation medium is not more than three-fold, preferably not more than two-fold, more preferably less than two-fold of the volume of the cultivation or incubation medium before the addition of said precursor and/or acceptor feed and wherein preferably, the pH of said precursor and/or acceptor feed is set between 2.0 and 10.0 and wherein preferably, the temperature of said precursor and/or acceptor feed is kept between 20° C. and 80° C.;
iv) Adding at least one precursor and/or acceptor feed in a continuous manner to the cultivation or incubation medium over the course of 1 day, 2 days, 3 days, 4 days, 5 days by means of a precursor and/or acceptor feeding solution;
v) Adding at least one precursor and/or acceptor feed in a continuous manner to the cultivation or incubation medium over the course of 1 day, 2 days, 3 days, 4 days, 5 days by means of a precursor and/or acceptor feeding solution and wherein preferably, the pH of said precursor and/or acceptor feeding solution is set between 2.0 and 10.0 and wherein preferably, the temperature of said precursor and/or acceptor feeding solution is kept between 20° C. and 80° C.;

said method resulting in a sialylated oligosaccharide, preferably a 6′sialylated oligosaccharide, more preferably LSTc, with a concentration of at least 50 g/L, preferably at least 75 g/L, more preferably at least 90 g/L, more preferably at least 100 g/L, more preferably at least 125 g/L, more preferably at least 150 g/L, more preferably at least 175 g/L, more preferably at least 200 g/L in the final volume of the cultivation or incubation. In a more preferred embodiment of the method of the invention, said precursor is chosen from the list comprising sialic acid, CMP-sialic acid and lactose. In another more preferred embodiment of the method of the invention, said acceptor is LNnT.

In another and/or additional preferred embodiment, the method for the production of a sialylated oligosaccharide as described herein comprises at least one of the following steps:
i) Use of a cultivation or incubation medium comprising at least one precursor and/or acceptor;
ii) Adding to the cultivation or incubation medium in a reactor or incubator at least one precursor and/or acceptor in one pulse or in a discontinuous (pulsed) manner wherein the total reactor or incubator volume ranges from 250 mL (millilitre) to 10.000 m³ (cubic meter), preferably so that the final volume of the cultivation or incubation medium is not more than three-fold, preferably not more than two-fold, more preferably less than two-fold of the volume of the cultivation or incubation medium before the addition of said precursor and/or acceptor feed pulse(s);
iii) Adding to the cultivation or incubation medium in a reactor or incubator at least one precursor and/or acceptor feed in one pulse or in a discontinuous (pulsed) manner wherein the total reactor or incubator volume ranges from 250 mL (millilitre) to 10.000 m³ (cubic meter), preferably so that the final volume of the cultivation or incubation medium is not more than three-fold, preferably not more than two-fold, more preferably less than two-fold of the volume of the cultivation or incubation medium before the addition of said precursor and/or acceptor feed and wherein preferably, the pH of said precursor and/or acceptor feed pulse(s) is set between 2.0 and 10.0 and wherein preferably, the temperature of said precursor and/or acceptor feed pulse(s) is kept between 20° C. and 80° C.;
iv) Adding at least one precursor and/or acceptor feed in a discontinuous (pulsed) manner to the cultivation or incubation medium over the course of 5 minutes, 10 minutes, 30 minutes, 1 hour, 2 hours, 4 hours, 10 hours, 12 hours, 1 day, 2 days, 3 days, 4 days, 5 days by means of a precursor and/or acceptor feeding solution;
v) Adding at least one precursor and/or acceptor feed in a discontinuous (pulsed) manner to the cultivation or incubation medium over the course of 5 minutes, 10 minutes, 30 minutes, 1 hour, 2 hours, 4 hours, 10 hours, 12 hours, 1 day, 2 days, 3 days, 4 days, 5 days by means of a precursor and/or acceptor feeding solution and wherein preferably, the pH of said precursor and/or acceptor feeding solution is set between 2.0 and 10.0 and wherein preferably, the temperature of said precursor and/or acceptor feeding solution is kept between 20° C. and 80° C.;
said method resulting in a sialylated oligosaccharide, preferably a 6′sialylated oligosaccharide, more preferably LSTc, with a concentration of at least 50 g/L, preferably at least 75 g/L, more preferably at least 90 g/L, more preferably at least 100 g/L, more preferably at least 125 g/L, more preferably at least 150 g/L, more preferably at least 175 g/L, more preferably at least 200 g/L in the final volume of the cultivation or incubation. In a more preferred embodiment of the method of the invention, said precursor is chosen from the list comprising sialic acid, CMP-sialic acid and lactose. In another more preferred embodiment of the method of the invention, said acceptor is LNnT.

In another and/or additional preferred embodiment, the method for the production of a sialylated oligosaccharide as described herein comprises at least one of the following steps:
i) Use of a cultivation or incubation medium comprising at least 50, more preferably at least 75, more preferably at least 100, more preferably at least 120, more preferably at least 150 grams of precursor per litre of initial reactor or incubator volume wherein the reactor or incubator volume ranges from 250 mL to 10.000 m³ (cubic meter);
ii) Adding to the cultivation or incubation medium in a reactor or incubator at least one precursor feed comprising at least 50, more preferably at least 75, more preferably at least 100, more preferably at least 120, more preferably at least 150 grams of precursor per litre of initial reactor or incubator volume wherein the total reactor or incubator volume ranges from 250 mL (millilitre) to 10.000 m³ (cubic meter), preferably in a continuous manner, and preferably so that the final volume of the cultivation or incubation medium is not more than three-fold, preferably not more than two-fold, more preferably less than two-fold of the volume of the cultivation or incubation medium before the addition of said precursor feed;
iii) Adding to the cultivation or incubation medium in a reactor or incubator at least one precursor feed comprising at least 50, more preferably at least 75, more preferably at least 100, more preferably at least 120, more preferably at least 150 grams of precursor per litre of initial reactor or incubator volume wherein the total reactor or incubator volume ranges from 250 mL (millilitre) to 10.000 m³ (cubic meter), preferably in a continuous manner, and preferably so that the final volume of the cultivation or incubation medium is not more than three-fold, preferably not more than two-fold, more preferably less than two-fold of the volume of the cultivation or incubation medium before the addition of said precursor feed and wherein preferably, the pH of said precursor feed is set between 2.0 and 10.0 and wherein preferably, the temperature of said precursor feed is kept between 20° C. and 80° C.;
iv) Adding at least one precursor feed in a continuous manner to the cultivation or incubation medium over the course of 1 day, 2 days, 3 days, 4 days, 5 days by means of a precursor feeding solution;
v) Adding at least one precursor feed in a continuous manner to the cultivation or incubation medium over the course of 1 day, 2 days, 3 days, 4 days, 5 days by means of a precursor feeding solution and wherein the concentration of said precursor feeding solution is 50 g/L, preferably 75 g/L, more preferably 100 g/L, more preferably 125 g/L, more preferably 150 g/L, more preferably 175 g/L, more preferably 200 g/L, more preferably 225 g/L, more preferably 250 g/L, more preferably 275 g/L, more preferably 300 g/L, more preferably 325 g/L, more preferably 350 g/L, more preferably 375 g/L, more preferably, 400 g/L, more preferably 450 g/L, more preferably 500 g/L, even more preferably, 550 g/L, most preferably 600 g/L; and wherein preferably, the pH of said precursor feeding solution is set between 2.0 and 10.0 and wherein preferably, the temperature of said precursor feeding solution is kept between 20° C. and 80° C.;

said method resulting in a sialylated oligosaccharide, preferably a 6′sialylated oligosaccharide, more preferably LSTc, with a concentration of at least 50 g/L, preferably at least 75 g/L, more preferably at least 90 g/L, more preferably at least 100 g/L, more preferably at least 125 g/L, more preferably at least 150 g/L, more preferably at least 175 g/L, more preferably at least 200 g/L in the final volume of the cultivation or incubation. In a more preferred embodiment of the method of the invention, said precursor is chosen from the list comprising sialic acid, CMP-sialic acid and lactose.

In another and/or additional preferred embodiment, the method for the production of a sialylated oligosaccharide as described herein comprises at least one of the following steps:
i) Use of a cultivation or incubation medium comprising at least 50, more preferably at least 75, more preferably at least 100, more preferably at least 120, more preferably at least 150 grams of acceptor per litre of initial reactor or incubator volume wherein the reactor or incubator volume ranges from 250 mL to 10.000 m³ (cubic meter);
ii) Adding to the cultivation or incubation medium in a reactor or incubator at least one acceptor feed comprising at least 50, more preferably at least 75, more preferably at least 100, more preferably at least 120, more preferably at least 150 grams of acceptor per litre of initial reactor or incubator volume wherein the total reactor or incubator volume ranges from 250 mL (millilitre) to 10.000 m³ (cubic meter), preferably in a continuous manner, and preferably so that the final volume of the cultivation or incubation medium is not more than three-fold, preferably not more than two-fold, more preferably less than two-fold of the volume of the cultivation or incubation medium before the addition of said acceptor feed;
iii) Adding to the cultivation or incubation medium in a reactor or incubator at least one acceptor feed comprising at least 50, more preferably at least 75, more preferably at least 100, more preferably at least 120, more preferably at least 150 grams of acceptor per litre of initial reactor or incubator volume wherein the total reactor or incubator volume ranges from 250 mL (millilitre) to 10.000 m³ (cubic meter), preferably in a continuous manner, and preferably so that the final volume of the cultivation or incubation medium is not more than three-fold, preferably not more than two-fold, more preferably less than two-fold of the volume of the cultivation or incubation medium before the addition of said acceptor feed and wherein preferably, the pH of said acceptor feed is set between 2.0 and 10.0 and wherein preferably, the temperature of said acceptor feed is kept between 20° C. and 80° C.;
iv) Adding at least one acceptor feed in a continuous manner to the cultivation or incubation medium over the course of 1 day, 2 days, 3 days, 4 days, 5 days by means of an acceptor feeding solution;
v) Adding at least one acceptor feed in a continuous manner to the cultivation or incubation medium over the course of 1 day, 2 days, 3 days, 4 days, 5 days by means of an acceptor feeding solution and wherein the concentration of said acceptor feeding solution is 50 g/L, preferably 75 g/L, more preferably 100 g/L, more preferably 125 g/L, more preferably 150 g/L, more preferably 175 g/L, more preferably 200 g/L, more preferably 225 g/L, more preferably 250 g/L, more preferably 275 g/L, more preferably 300 g/L, more preferably 325 g/L, more preferably 350 g/L, more preferably 375 g/L, more preferably, 400 g/L, more preferably 450 g/L, more preferably 500 g/L, even more preferably, 550 g/L, most preferably 600 g/L; and wherein preferably, the pH of said acceptor feeding solution is set between 2.0 and 10.0 and wherein preferably, the temperature of said acceptor feeding solution is kept between 20° C. and 80° C.;

said method resulting in a sialylated oligosaccharide, preferably a 6′sialylated oligosaccharide, more preferably LSTc, with a concentration of at least 50 g/L, preferably at least 75 g/L, more preferably at least 90 g/L, more preferably at least 100 g/L, more preferably at least 125 g/L, more preferably at least 150 g/L, more preferably at least 175 g/L, more preferably at least 200 g/L in the final volume of the cultivation or incubation. In a more preferred embodiment of the method of the invention, said acceptor is LNnT.

In another and/or additional preferred embodiment, the method for the production of a sialylated oligosaccharide as described herein comprises at least one of the following steps:
i) Use of a cultivation or incubation medium comprising at least 50, more preferably at least 75, more preferably at least 100, more preferably at least 120, more preferably at least 150 grams of precursor per litre of initial reactor or incubator volume wherein the reactor or incubator volume ranges from 250 mL to 10.000 m³ (cubic meter);
ii) Adding to the cultivation or incubation medium in a reactor or incubator at least one precursor feed comprising at least 50, more preferably at least 75, more preferably at least 100, more preferably at least 120, more preferably at least 150 grams of precursor per litre of initial reactor or incubator volume wherein the total reactor or incubator volume ranges from 250 mL (millilitre) to 10.000 m³ (cubic meter) in one pulse or in a discontinuous (pulsed), preferably so that the final volume of the cultivation or incubation medium is not more than three-fold, preferably not more than two-fold, more preferably less than two-fold of the volume of the cultivation or incubation medium before the addition of said precursor feed pulse(s);
iii) Adding to the cultivation or incubation medium in a reactor or incubator at least one precursor feed comprising at least 50, more preferably at least 75, more preferably at least 100, more preferably at least 120, more preferably at least 150 grams of precursor per litre of initial reactor or incubator volume wherein the total reactor or incubator volume ranges from 250 mL (millilitre) to 10.000 m³ (cubic meter) in one pulse or in a discontinuous (pulsed) manner, preferably so that the final volume of the cultivation or incubation medium is not more than three-fold, preferably not more than two-fold, more preferably less than two-fold of the volume of the cultivation or incubation medium before the addition of said precursor feed and wherein preferably, the pH of said precursor feed pulse(s) is set between 2.0 and 10.0 and wherein preferably, the temperature of said precursor feed pulse(s) is kept between 20° C. and 80° C.;
iv) Adding at least one precursor feed in a discontinuous (pulsed) manner to the cultivation or incubation medium over the course of 5 minutes, 10 minutes, 30 minutes, 1 hour, 2 hours, 4 hours, 10 hours, 12 hours, 1 day, 2 days, 3 days, 4 days, 5 days by means of a precursor feeding solution;
v) Adding at least one precursor feed in a discontinuous (pulsed) manner to the cultivation or incubation medium over the course of 5 minutes, 10 minutes, 30 minutes, 1 hour, 2 hours, 4 hours, 10 hours, 12 hours, 1 day, 2 days, 3 days, 4 days, 5 days by means of a precursor feeding solution and wherein the concentration of said precursor feeding solution is 50 g/L, preferably 75 g/L, more preferably 100 g/L, more preferably 125 g/L, more preferably 150 g/L, more preferably 175 g/L, more preferably 200 g/L, more preferably 225 g/L, more preferably 250 g/L, more preferably 275 g/L, more preferably 300 g/L, more preferably 325 g/L, more preferably 350 g/L, more preferably 375 g/L, more preferably, 400 g/L, more preferably 450 g/L, more preferably 500 g/L, even more preferably, 550 g/L, most preferably 600 g/L; and wherein preferably, the pH of said precursor feeding solution is set between 2.0 and 10.0 and wherein preferably, the temperature of said precursor feeding solution is kept between 20° C. and 80° C.;

said method resulting in a sialylated oligosaccharide, preferably a 6′sialylated oligosaccharide, more preferably LSTc, with a concentration of at least 50 g/L, preferably at least 75 g/L, more preferably at least 90 g/L, more preferably at least 100 g/L, more preferably at least 125 g/L, more preferably at least 150 g/L, more preferably at least 175 g/L, more preferably at least 200 g/L in the final volume of the cultivation or incubation. In a more preferred embodiment of the method of the invention, said precursor is chosen from the list comprising sialic acid, CMP-sialic acid and lactose.

In another and/or additional preferred embodiment, the method for the production of a sialylated oligosaccharide as described herein comprises at least one of the following steps:
i) Use of a cultivation or incubation medium comprising at least 50, more preferably at least 75, more preferably at least 100, more preferably at least 120, more preferably at least 150 grams of acceptor per litre of initial reactor or incubator volume wherein the reactor or incubator volume ranges from 250 mL to 10.000 m³ (cubic meter);
ii) Adding to the cultivation or incubation medium in a reactor or incubator at least one acceptor feed comprising at least 50, more preferably at least 75, more preferably at least 100, more preferably at least 120, more preferably at least 150 grams of acceptor per litre of initial reactor or incubator volume wherein the total reactor or incubator volume ranges from 250 mL (millilitre) to 10.000 m³ (cubic meter) in one pulse or in a discontinuous (pulsed), preferably so that the final volume of the cultivation or incubation medium is not more than three-fold, preferably not more than two-fold, more preferably less than two-fold of the volume of the cultivation or incubation medium before the addition of said acceptor feed pulse(s);
iii) Adding to the cultivation or incubation medium in a reactor or incubator at least one acceptor feed comprising at least 50, more preferably at least 75, more preferably at least 100, more preferably at least 120, more preferably at least 150 grams of acceptor per litre of initial reactor or incubator volume wherein the total reactor or incubator volume ranges from 250 mL (millilitre) to 10.000 m³ (cubic meter) in one pulse or in a discontinuous (pulsed) manner, preferably so that the final volume of the cultivation or incubation medium is not more than three-fold, preferably not more than two-fold, more preferably less than two-fold of the volume of the cultivation or incubation medium before the addition of said acceptor feed and wherein preferably, the pH of said acceptor feed pulse(s) is set between 2.0 and 10.0 and wherein preferably, the temperature of said acceptor feed pulse(s) is kept between 20° C. and 80° C.;
iv) Adding at least one acceptor feed in a discontinuous (pulsed) manner to the cultivation or incubation medium over the course of 5 minutes, 10 minutes, 30 minutes, 1 hour, 2 hours, 4 hours, 10 hours, 12 hours, 1 day, 2 days, 3 days, 4 days, 5 days by means of an acceptor feeding solution;
v) Adding at least one acceptor feed in a discontinuous (pulsed) manner to the cultivation or incubation medium over the course of 5 minutes, 10 minutes, 30 minutes, 1 hour, 2 hours, 4 hours, 10 hours, 12 hours, 1 day, 2 days, 3 days, 4 days, 5 days by means of an acceptor feeding solution and wherein the concentration of said acceptor feeding solution is 50 g/L, preferably 75 g/L, more preferably 100 g/L, more preferably 125 g/L, more preferably 150 g/L, more preferably 175 g/L, more preferably 200 g/L, more preferably 225 g/L, more preferably 250 g/L, more preferably 275 g/L, more preferably 300 g/L, more preferably 325 g/L, more preferably 350 g/L, more preferably 375 g/L, more preferably, 400 g/L, more preferably 450 g/L, more preferably 500 g/L, even more preferably, 550 g/L, most preferably 600 g/L; and wherein preferably, the pH of said acceptor feeding solution is set between 2.0 and 10.0 and wherein preferably, the temperature of said acceptor feeding solution is kept between 20° C. and 80° C.;

said method resulting in a sialylated oligosaccharide, preferably a 6′sialylated oligosaccharide, more preferably LSTc, with a concentration of at least 50 g/L, preferably at least 75 g/L, more preferably at least 90 g/L, more preferably at least 100 g/L, more preferably at least 125 g/L, more preferably at least 150 g/L, more preferably at least 175 g/L, more preferably at least 200 g/L in the final volume of the cultivation or incubation. In a more preferred embodiment of the method of the invention, said acceptor is LNnT.

In a more preferred embodiment, the method for the production of a sialylated oligosaccharide as described herein comprises at least one of the following steps:
i) Use of a cultivation or incubation medium comprising at least 50, more preferably at least 75, more preferably at least 100, more preferably at least 120, more preferably at least 150 grams of lactose per litre of initial reactor or incubator volume wherein the reactor or incubator volume ranges from 250 mL to 10.000 m³ (cubic meter);
ii) Adding to the cultivation or incubation medium in a reactor or incubator a lactose feed comprising at least 50, more preferably at least 75, more preferably at least 100, more preferably at least 120, more preferably at least 150 gram of lactose per litre of initial reactor or incubator volume wherein the total reactor or incubator volume ranges from 250 mL (millilitre) to 10.000 m³ (cubic meter), preferably in a continuous manner, and preferably so that the final volume of the cultivation or incubation medium is not more than three-fold, preferably not more than two-fold, more preferably less than two-fold of the volume of the cultivation or incubation medium before the addition of said lactose feed;
iii) Adding to the cultivation or incubation medium in a reactor or incubator a lactose feed comprising at least 50, more preferably at least 75, more preferably at least 100, more preferably at least 120, more preferably at least 150 grams of lactose per litre of initial reactor or incubator volume wherein the total reactor or incubator volume ranges from 250 mL (millilitre) to 10.000 m³ (cubic meter), preferably in a continuous manner, and preferably so that the final volume of the cultivation or incubation medium is not more than three-fold, preferably not more than two-fold, more preferably less than two-fold of the volume of the cultivation or incubation medium before the addition of said lactose feed and wherein preferably, the pH of said lactose feed is set between 2.0 and 10.0, preferably between 3.0 and 7.0, and wherein preferably, the temperature of said lactose feed is kept between 20° C. and 80° C.;
iv) Adding a lactose feed in a continuous manner to the cultivation or incubation medium over the course of 1 day, 2 days, 3 days, 4 days, 5 days by means of a feeding solution;
v) Adding a lactose feed in a continuous manner to the cultivation or incubation medium over the course of 1 day, 2 days, 3 days, 4 days, 5 days by means of a feeding solution and wherein the concentration of said lactose feeding solution is 50 g/L, preferably 75 g/L, more preferably 100 g/L, more preferably 125 g/L, more preferably 150 g/L, more preferably 175 g/L, more preferably 200 g/L, more preferably 225 g/L, more preferably 250 g/L, more preferably 275 g/L, more preferably 300 g/L, more preferably 325 g/L, more preferably 350 g/L, more preferably 375 g/L, more preferably, 400 g/L, more preferably 450 g/L, more preferably 500 g/L, even more preferably, 550 g/L, most preferably 600 g/L; and wherein preferably, the pH of said lactose feed is set between 2.0 and 10.0, preferably between 3.0 and 7.0 and wherein preferably, the temperature of said lactose feed is kept between 20° C. and 80° C.;

said method resulting in a sialylated oligosaccharide, preferably a 6′sialylated oligosaccharide, more preferably LSTc, with a concentration of at least 50 g/L, preferably at least 75 g/L, more preferably at least 90 g/L, more preferably at least 100 g/L, more preferably at least 125 g/L, more preferably at least 150 g/L, more preferably at least 175 g/L, more preferably at least 200 g/L in the final volume of the cultivation or incubation.

Preferably the lactose feed is accomplished by adding lactose from the beginning of the cultivation or incubation in a concentration of at least 5 mM, preferably in a concentration of 30, 40, 50, 60, 70, 80, 90, 100, 150 mM, more preferably in a concentration>300 mM.

In another embodiment of the methods, the lactose feed is accomplished by adding lactose to the cultivation or incubation medium in a concentration, such that throughout the production phase of the cultivation or incubation a lactose concentration of at least 5 mM, preferably 10 mM or 30 mM is obtained.

In a further embodiment of the methods described herein the cells are cultivated or incubated for at least about 60, 80, 100, or about 120 hours or in a continuous manner.

In a preferred embodiment, a carbon source is provided, preferably sucrose, in the cultivation medium for 3 or more days, preferably up to 7 days; and/or provided, in the cultivation medium, at least 100, advantageously at least 105, more advantageously at least 110, even more advantageously at least 120 grams of sucrose per litre of initial cultivation volume in a continuous manner, so that the final volume of the cultivation medium is not more than three-fold, advantageously not more than two-fold, more advantageously less than two-fold of the volume of the cultivation medium before the cultivation.

Preferably, when performing the method as described herein, a first phase of exponential cell growth is provided by adding a carbon source, preferably glucose or sucrose, to the cultivation medium before the lactose is added to the cultivation medium in a second phase.

In an alternative preferable embodiment, in the method as described herein, the lactose is added already in the first phase of exponential growth together with the carbon-based substrate.

According to another aspect, the present invention provides a metabolically engineered cell for the production of a sialylated oligosaccharide, preferably a 6′sialylated oligosaccharide, more preferably LSTc (Neu5Ac-α2,6-Gal-β1,4-GlcNAc-β1,3-Gal-β1,4-Glc), wherein said cell has been metabolically engineered to possess, preferably to express, more preferably to heterologously express, even more preferably to overexpress, most preferably to heterologously overexpress, an alpha-2,6-sialyltransferase which has alpha-2,6-sialyltransferase activity on the terminal Gal residue of LNnT and comprises an amino acid sequence that is at least 60.0% identical over a stretch of at least 100 amino acid residues, preferably at least 150 amino acid residues, more preferably at least 200 amino acid residues, to the amino acid sequence as represented by SEQ ID NO 1. Preferably, said alpha-2,6-sialyltransferase used in the cell is an alpha-2,6-sialyltransferase as described herein.

Alternatively or preferably, the cell contains a nucleic acid molecule which comprises a polynucleotide sequence which encodes any one of the alpha-2,6-sialyltransferases as described herein.

Herein, a metabolically engineered cell comprising a pathway for production of said sialylated oligosaccharide is provided. Examples of such pathways comprise but are not limited to pathways involved in the synthesis of monosaccharide, phosphorylated monosaccharide, nucleotide-activated sugar, and/or glycosylation pathways like e.g. a fucosylation, sialylation, galactosylation, N-acetylglucosaminylation, N-acetylgalactosylation, mannosylation and/or N-acetylmannosaminylation pathway. Said pathway for production of a sialylated oligosaccharide preferably comprises at least one alpha-2,6-sialyltransferase as described herein.

In a preferred embodiment of the method and/or cell of present invention, the cell comprises one or more pathway(s) for monosaccharide synthesis. Said pathways for monosaccharide synthesis comprise enzymes like e.g. carboxylases, decarboxylases, isomerases, epimerases, reductases, enolases, phosphorylases, carboxykinases, kinases, phosphatases, aldolases, hydrolases, dehydrogenases, enzymes involved in the synthesis of one or more nucleoside triphosphate(s) like UTP, GTP, ATP and CTP, enzymes involved in the synthesis of any one or more nucleoside mono- or diphosphates like e.g. UMP and UDP, respectively, and enzymes involved in the synthesis of phosphoenolpyruvate (PEP).

In another and/or additional preferred embodiment of the method and/or cell of present invention, the cell comprises one or more pathway(s) for phosphorylated monosaccharide synthesis. Said pathways for phosphorylated monosaccharide synthesis comprise enzymes involved in the synthesis of one or more monosaccharide(s), one or more nucleoside mono-, di- and/or triphosphate(s) and enzymes involved in the synthesis of phosphoenolpyruvate (PEP) like e.g. but not limited to PEP synthase, carboxylases, decarboxylases, isomerases, epimerases, reductases, enolases, phosphorylases, carboxykinases, kinases, phosphatases, aldolases, hydrolases and dehydrogenases. In another and/or additional preferred embodiment of the method and/or cell of present invention, the cell comprises one or more pathways for the synthesis of one or more nucleotide-activated sugars. Said pathways for nucleotide-activated sugar synthesis comprise enzymes like e.g. PEP synthase, carboxylases, decarboxylases, isomerases, epimerases, reductases, enolases, phosphorylases, carboxykinases, kinases, phosphatases, aldolases, hydrolases, dehydrogenases, mannose-6-phosphate isomerase, phosphomannomutase, mannose-1-phosphate guanylyltransferase, GDP-mannose 4,6-dehydratase, GDP-L-fucose synthase, L-fucokinase/GDP-fucose pyrophosphorylase, L-glutamine-D-fructose-6-phosphate aminotransferase, glucosamine-6-phosphate deaminase, phosphoglucosamine mutase, N-acetylglucosamine-6-phosphate deacetylase, N-acylglucosamine 2-epimerase, UDP-N-acetylglucosamine 2-epimerase, N-acetylglucosamine-6P 2-epimerase, glucosamine 6-phosphate N-acetyltransferase, N-acetylglucosamine-6-phosphate phosphatase, N-acetylmannosamine-6-phosphate 2-epimerase, N-acetylmannosamine-6-phosphate phosphatase, N-acetylmannosamine kinase, phosphoacetylglucosamine mutase, N-acetylglucosamine-1-phosphate uridylyltransferase, glucosamine-1-phosphate acetyltransferase, sialic acid synthase, N-acetylneuraminate lyase, N-acylneuraminate-9-phosphate synthase, N-acylneuraminate-9-phosphatase, CMP-sialic acid synthase, galactose-1-epimerase, galactokinase, glucokinase, galactose-1-phosphate uridylyltransferase, UDP-glucose 4-epimerase, glucose-1-phosphate uridylyltransferase and/or phosphoglucomutase.

Said cell may further comprise and express at least one further glycosyltransferase that is involved in the production of said sialylated oligosaccharide.

In a preferred embodiment of the method and/or cell of present invention, the cell is metabolically engineered to comprise a pathway for production of a sialylated oligosaccharide as defined herein, preferably for production of a 6′sialylated oligosaccharide, more preferably LSTc, as defined herein. In an alternative preferred embodiment of the method and/or cell of present invention, the cell is metabolically engineered to comprise a pathway for production of a sialylated oligosaccharide as defined herein, preferably for production of a 6′sialylated oligosaccharide, preferably LSTc, and to have modified expression or activity of an alpha-2,6-sialyltransferase of present invention.

In a further preferred embodiment of the method and/or cell of present invention, the cell comprises a recombinant alpha-2,6-sialyltransferase capable of modifying LNnT or another acceptor as defined herein with one or more sialic acid molecules that is/are synthesized by any one or more Neu5Ac synthases expressed in the cell, into a sialylated oligosaccharide, preferably a 6′sialylated oligosaccharide, more preferably LSTc.

In a preferred embodiment of the method and/or cell of the invention, the metabolically engineered cell is modified with one or more expression modules.

Said expression modules are also known as transcriptional units and comprise polynucleotides for expression of recombinant genes including coding gene sequences and appropriate transcriptional and/or translational control signals that are operably linked to the coding genes. Said control signals comprise promoter sequences, untranslated regions, ribosome binding sites, terminator sequences. Said expression modules can contain elements for expression of one single recombinant gene but can also contain elements for expression of more recombinant genes or can be organized in an operon structure for integrated expression of two or more recombinant genes. Said polynucleotides may be produced by recombinant DNA technology using techniques well-known in the art. Methods which are well known to those skilled in the art to construct expression modules include, for example, in vitro recombinant DNA techniques, synthetic techniques, and in vivo genetic recombination. See, for example, the techniques described in Sambrook et al. (2001) Molecular Cloning: a laboratory manual, 3rd Edition, Cold Spring Harbor Laboratory Press, CSH, New York or to Current Protocols in Molecular Biology, John Wiley and Sons, N.Y. (1989 and yearly updates).

The expression of each of said expression modules can be constitutive or is created by a natural or chemical inducer. As used herein, constitutive expression should be understood as expression of a gene that is transcribed continuously in an organism. Expression that is created by a natural inducer should be understood as a facultative or regulatory expression of a gene that is only expressed upon a certain natural condition of the host (e.g. organism being in labour, or during lactation), as a response to an environmental change (e.g. including but not limited to hormone, heat, cold, pH shifts, light, oxidative or osmotic stress/signalling), or dependent on the position of the developmental stage or the cell cycle of said host cell including but not limited to apoptosis and autophagy. Expression that is created by a chemical inducer should be understood as a facultative or regulatory expression of a gene that is only expressed upon sensing of external chemicals (e.g. IPTG, arabinose, lactose, allo-lactose, rhamnose or fucose) via an inducible promoter or via a genetic circuit that either induces or represses the transcription or translation of said polynucleotide to a polypeptide.

The expression modules can be integrated in the genome of said cell or can be presented to said cell on a vector. Said vector can be present in the form of a plasmid, cosmid, phage, liposome, or virus, which is to be stably transformed/transfected into said metabolically engineered cell. Such vectors include, among others, chromosomal, episomal and virus-derived vectors, e.g. vectors derived from bacterial plasmids, from bacteriophage, from transposons, from yeast episomes, from insertion elements, from yeast chromosomal elements, from viruses, and vectors derived from combinations thereof, such as those derived from plasmid and bacteriophage genetic elements, such as cosmids and phagemids. These vectors may contain selection markers such as but not limited to antibiotic markers, auxotrophic markers, toxin-antitoxin markers, RNA sense/antisense markers. The expression system constructs may contain control regions that regulate as well as engender expression. Generally, any system or vector suitable to maintain, propagate or express polynucleotides and/or to express a polypeptide in a host may be used for expression in this regard. The appropriate DNA sequence may be inserted into the expression system by any of a variety of well-known and routine techniques, such as, for example, those set forth in Sambrook et al., see above. For recombinant production, cells can be genetically engineered to incorporate expression systems or portions thereof or polynucleotides of the invention. Introduction of a polynucleotide into the cell can be effected by methods described in many standard laboratory manuals, such as Davis et al., Basic Methods in Molecular Biology, (1986), and Sambrook et al., 1989, supra.

As used herein an expression module comprises polynucleotides for expression of at least one recombinant gene. Said recombinant gene is involved in the expression of a polypeptide acting in the synthesis of a sialylated oligosaccharide, such as e.g. LSTc; or said recombinant gene is linked to other pathways in said cell that are not involved in the synthesis of a sialylated oligosaccharide. Said recombinant genes encode endogenous proteins with a modified expression or activity, preferably said endogenous proteins are overexpressed; or said recombinant genes encode heterologous proteins that are heterogeneously introduced and expressed in said modified cell, preferably overexpressed. The endogenous proteins can have a modified expression in the cell which also expresses a heterologous protein.

In a preferred embodiment of the method and/or cell of the invention, the expression of each of said expression modules present in said metabolically engineered cell is constitutive or tuneable as described herein.

In a further embodiment of the method and/or cell of the invention, the cell is modified in the expression or activity of at least one of said alpha-2,6-sialyltransferases. In a preferred embodiment, said alpha-2,6-sialyltransferase is an endogenous protein of the cell with a modified expression or activity, preferably said endogenous alpha-2,6-sialyltransferase is overexpressed; alternatively said alpha-2,6-sialyltransferase is a heterologous protein that is heterogeneously introduced and expressed in said cell, preferably overexpressed. Said endogenous alpha-2,6-sialyltransferase can have a modified expression in the cell which also expresses a heterologous alpha-2,6-sialyltransferase.

According to a preferred embodiment of the method and/or cell of the invention, the cell comprises a pathway for production of a sialylated oligosaccharide, preferably a 6′sialylated oligosaccharide, more preferably LSTc, comprising at least one alpha-2,6-sialyltransferase according to present invention. According to another preferred embodiment of the method and/or cell of the invention, said pathway for production of a sialylated oligosaccharide, preferably a 6′sialylated oligosaccharide, more preferably LSTc, further comprises at least one enzyme chosen from the list comprising L-glutamine-D-fructose-6-phosphate aminotransferase, a phosphoglucosamine mutase, an N-acetylglucosamine-6-P deacetylase, an N-acylglucosamine 2-epimerase, a UDP-N-acetylglucosamine 2-epimerase, an N-acetylmannosamine-6-phosphate 2-epimerase, a UDP-GlcNAc 2-epimerase/kinase, a glucosamine 6-phosphate N-acetyltransferase, an N-acetylglucosamine-6-phosphate phosphatase, a phosphoacetylglucosamine mutase, an N-acetylglucosamine 1-phosphate uridylyltransferase, a glucosamine-1-phosphate acetyltransferase, an Neu5Ac synthase, an N-acetylneuraminate lyase, an N-acylneuraminate-9-phosphate synthase, an N-acylneuraminate-9-phosphatase, a sialic acid transporter and a CMP-sialic acid synthase.

In a preferred embodiment, the cell comprises a pathway for production of a sialylated oligosaccharide, preferably a 6′sialylated oligosaccharide, more preferably LSTc, wherein said cell expresses at least one enzyme chosen from the list comprising an N-acylglucosamine 2-epimerase like is known e.g. from several species including Bacteroides ovatus, E. coli, Homo sapiens, Rattus norvegicus, a Neu5Ac synthase, a CMP sialic acid synthase like is known e.g. from Neisseria meningitidis, and an alpha-2,6-sialyltransferase according to present invention, wherein the enzymes are as defined herein. N-acetylglucosamine (GlcNAc) can be added to the cell and/or can be provided by an enzyme expressed in the cell or by the mechanism of the cell. Such cell producing GlcNAc can express a phosphatase converting GlcNAc-6-phosphate into GlcNAc, like any one or more of e.g. the E. coli HAD-like phosphatase genes comprising aphA, Cof, HisB, OtsB, SurE, Yaed, YcjU, YedP, YfbT, YidA, YigB, YihX, YniC, YqaB, YrbL, AppA, Gph, SerB, YbhA, YbiV, YbjL, Yfb, YieH, YjgL, YjjG, YrfG and YbiU, PsMupP from Pseudomonas putida, ScDOG1 from S. cerevisiae and BsAraL from Bacillus subtilis as described in WO 2018/122225. Preferably, the cell is modified to produce GlcNAc. More preferably, the cell is modified for enhanced GlcNAc production. Said modification can be any one or more chosen from the group comprising knockout of a glucosamine-6-phosphate deaminase, an N-acetylglucosamine-6-phosphate deacetylase and/or an N-acetyl-D-glucosamine kinase and over-expression of an L-glutamine-D-fructose-6-phosphate aminotransferase and/or a glucosamine 6-phosphate N-acetyltransferase.

In an alternative and/or additional preferred embodiment, the cell comprises a pathway for production of a sialylated oligosaccharide, preferably a 6′sialylated oligosaccharide, more preferably LSTc, wherein said cell expresses at least one enzyme chosen from the list comprising a UDP-N-acetylglucosamine 2-epimerase like is known e.g. from several species including Campylobacter jejuni, E. coli, Neisseria meningitidis, Bacillus subtilis, Citrobacter rodentium, a Neu5Ac synthase, a CM P sialic acid synthase like is known e.g from Neisseria meningitidis, and an alpha-2,6-sialyltransferase according to present invention, wherein the enzymes are as defined herein. UDP-N-acetylglucosamine (UDP-GlcNAc) can be added to the cell and/or can be provided by an enzyme expressed in the cell or by the metabolism of the cell. Such cell producing a UDP-GlcNAc can express enzymes converting, e.g. GlcNAc, which is to be added to the cell, to UDP-GlcNAc. These enzymes may be any one or more enzymes chosen from the list comprising an N-acetyl-D-glucosamine kinase, an N-acetylglucosamine-6-phosphate deacetylase, a phosphoglucosamine mutase, and an N-acetylglucosamine-1-phosphate uridyltransferase/glucosamine-1-phosphate acetyltransferase from several species including Homo sapiens, Escherichia coli. Preferably, the cell is modified to produce UDP-GlcNAc. More preferably, the cell is modified for enhanced UDP-GlcNAc production. Said modification can be any one or more chosen from the group comprising knock-out of an N-acetylglucosamine-6-phosphate deacetylase, over-expression of an L-glutamine-D-fructose-6-phosphate aminotransferase, over-expression of a phosphoglucosamine mutase, and over-expression of an N-acetylglucosamine-1-phosphate uridylyltransferase/glucosamine-1-phosphate acetyltransferase.

In an alternative and/or additional preferred embodiment, the cell comprises a pathway for production of a sialylated oligosaccharide, preferably a 6′sialylated oligosaccharide, more preferably LSTc, wherein said cell expresses at least one enzyme chosen from the list comprising an N-acetylmannosamine-6-phosphate 2-epimerase like is known e.g. from several species including E. coli, Haemophilus influenzae, Enterobacter sp., Streptomyces sp., an N-acylneuraminate-9-phosphate synthetase, an N-acylneuraminate-9-phosphatase like is known e.g. from Candidatus magnetomorum sp. HK-1 or Bacteroides thetaiotaomicron, a Neu5Ac synthase, a CMP sialic acid synthase like is known e.g. from Neisseria meningitidis, and an alpha-2,6-sialyltransferase according to present invention, wherein the enzymes are as defined herein. N-acetyl-D-glucosamine 6-phosphate (GlcNAc-6P) can be added to the cell and/or can be provided by an enzyme expressed in the cell or by the metabolism of the cell. Such cell producing GlcNAc-6P can express an enzyme converting, e.g. GlcN6P, which is to be added to the cell, to GlcNAc-6P. This enzyme may be a glucosamine 6-phosphate N-acetyltransferase from several species including Saccharomyces cerevisiae, Kluyveromyces lactis, Homo sapiens. Preferably, the cell is modified to produce GlcNAc-6P. More preferably, the cell is modified for enhanced GlcNAc-6P production. Said modification can be any one or more chosen from the group comprising knockout of a glucosamine-6-phosphate deaminase, an N-acetylglucosamine-6-phosphate deacetylase and over-expression of an L-glutamine-D-fructose-6-phosphate aminotransferase and/or a glucosamine 6-phosphate N-acetyltransferase.

In an alternative and/or additional preferred embodiment, the cell comprises a pathway for production of a sialylated oligosaccharide, preferably a 6′sialylated oligosaccharide, more preferably LSTc, wherein said cell expresses at least one enzyme chosen from the list comprising a bifunctional UDP-GlcNAc 2-epimerase/kinase like is known e.g. from several species including Homo sapiens, Rattus norvegicus and Mus musculus, an N-acylneuraminate-9-phosphate synthetase, an N-acylneuraminate-9-phosphatase like is known e.g. from Candidatus magnetomorum sp. HK-1 or Bacteroides thetaiotaomicron, a Neu5Ac synthase, a CMP sialic acid synthase like is known e.g. from Neisseria meningitidis, and an alpha-2,6-sialyltransferase according to present invention, wherein the enzymes are as defined herein. UDP-N-acetylglucosamine can be added to the cell and/or can be provided by an enzyme expressed in the cell or by the metabolism of the cell. Such cell producing a UDP-GlcNAc can express enzymes converting, e.g. GlcNAc, which is to be added to the cell, to UDP-GlcNAc. These enzymes may be an N-acetyl-D-glucosamine kinase, an N-acetylglucosamine-6-phosphate deacetylase, a phosphoglucosamine mutase, and an N-acetylglucosamine-1-phosphate uridylyltransferase/glucosamine-1-phosphate acetyltransferase from several species including Homo sapiens, Escherichia coli. Preferably, the cell is modified to produce UDP-GlcNAc. More preferably, the cell is modified for enhanced UDP-GlcNAc production. Said modification can be any one or more chosen from the group comprising knock-out of an N-acetylglucosamine-6-phosphate deacetylase, over-expression of an L-glutamine-D-fructose-6-phosphate aminotransferase, over-expression of a phosphoglucosamine mutase, and over-expression of an N-acetylglucosamine-1-phosphate uridylyltransferase/glucosamine-1-phosphate acetyltransferase.

Additionally, or alternatively, the cell used herein is optionally genetically engineered to import a precursor and/or an acceptor in the cell, by the introduction and/or overexpression of a transporter able to import the respective precursor and/or acceptor in the cell. Such transporter is for example a membrane protein belonging to the major facilitator superfamily (MFS), the ATP-binding cassette (ABC) transporter family or the PTS system involved in the uptake of e.g. mono-, di- and/or oligosaccharides.

Additionally, or alternatively, the cell used herein is optionally genetically engineered to produce polyisoprenoid alcohols like e.g. phosphorylated dolichol that can act as lipid carrier.

Additionally, or alternatively, the cell used herein is optionally genetically engineered to import lactose in the cell, by the introduction and/or overexpression of a lactose permease. Said lactose permease is for example encoded by the lacY gene or the lac12 gene.

Additionally, or alternatively, the cell expresses a membrane protein that is a transporter protein involved in transport of compounds and/or a sialylated oligosaccharide as defined in present invention out of the cell. Preferably the cell is transformed to comprise at least one nucleic acid sequence encoding a protein selected from the group comprising a lactose transporter like e.g. the LacY or lac12 permease, a glucose transporter, a galactose transporter, a transporter for a nucleotide-activated sugar like for example a transporter for UDP-GlcNAc, a transporter protein involved in transport of a sialylated oligosaccharide, preferably a 6′sialylated oligosaccharide, more preferably LSTc, out of the cell. Preferably the cell is transformed to comprise at least one nucleic acid sequence encoding a membrane transporter protein selected from the group comprising a siderophore exporter, a major facilitator superfamily (MFS) transporter, an ATP-binding cassette (ABC) transporter or a sugar efflux transporter.

According to another preferred aspect of the method and/or cell of the present invention, the cell is capable to synthesize N-acetylmannosamine (ManNAc), N-acetylmannosamine-6-phosphate (ManNAc-6-phosphate) and/or phosphoenolpyruvate (PEP).

In a preferred embodiment, the cell comprises a pathway for production of a sialylated oligosaccharide, preferably a 6′sialylated oligosaccharide, more preferably LSTc, comprising a pathway for production of ManNAc. ManNAc can be provided by an enzyme expressed in the cell or by the mechanism of the cell.

Such cell producing ManNAc can express an N-acylglucosamine 2-epimerase like is known e.g. from several species including Bacteroides ovatus, E. coli, Homo sapiens, Rattus norvegicus that converts GlcNAc into ManNAc. Alternatively, and/or additionally, the cell producing ManNAc can express a UDP-N-acetylglucosamine 2-epimerase like is known e.g. from several species including Campylobacter jejuni, E. coli, Neisseria meningitidis, Bacillus subtilis, Citrobacter rodentium that converts UDP-GlcNAc into ManNAc. GlcNAc and/or UDP-GlcNAc can be added to the cell and/or provided by an enzyme expressed in the cell or by the mechanism of the cell as described herein.

In a more preferred embodiment, the cell is modified for enhanced ManNAc production. Said modification can be any one or more chosen from the group comprising knock-out of N-acetylmannosamine kinase, over-expression of N-acetylneuraminate lyase.

In another preferred embodiment, the cell comprises a pathway for production of a sialylated oligosaccharide, preferably a 6′sialylated oligosaccharide, more preferably LSTc, comprising a pathway for production of ManNAc-6-phosphate. ManNAc-6-phosphate can be provided by an enzyme expressed in the cell or by the mechanism of the cell. Such cell producing ManNAc-6-phosphate can express a bifunctional UDP-GlcNAc 2-epimerase/kinase like is known e.g. from several species including Homo sapiens, Rattus norvegicus and Mus musculus that converts UDP-GlcNAc into ManNAc-6-phosphate.

Alternatively, and/or additionally, the cell producing ManNAc-6-phosphate can express an N-acetylmannosamine-6-phosphate 2-epimerase that converts GlcNAc-6-phosphate into ManNAc-6-phosphate. UDP-GlcNAc and/or GlcNAc-6-phosphate can be added to the cell and/or provided by an enzyme expressed in the cell or by the mechanism of the cell as described herein. In a more preferred embodiment, the cell is modified for enhanced ManNAc-6-phosphate production. Said modification can be any one or more chosen from the group comprising over-expression of N-acetylglucosamine-6-phosphate deacetylase, over-expression of N-acetyl-D-glucosamine kinase, over-expression of phosphoglucosamine mutase, over-expression of N-acetylglucosamine-1-phosphate uridyltransferase/glucosamine-1-phosphate acetyltransferase.

According to another embodiment of the method and/or cell of the invention, the cell is further capable to synthesize any one or more nucleotide-activated sugars. In a preferred embodiment of the method and/or cell of the invention, the cell is capable to synthesize one or more nucleotide-activated sugars chosen from the list comprising UDP-N-acetylglucosamine (UDP-GlcNAc), UDP-N-acetylgalactosamine (UDP-GalNAc), UDP-N-acetylmannosamine (UDP-ManNAc), UDP-glucose (UDP-Glc), UDP-galactose (UDP-Gal), GDP-mannose (GDP-Man), UDP-glucuronate, UDP-galacturonate, UDP-2-acetamido-2,6-dideoxy-L-arabino-4-hexulose, UDP-2-acetamido-2,6-dideoxy-L-lyxo-4-hexulose, UDP-N-acetyl-L-rhamnosamine (UDP-L-RhaNAc or UDP-2-acetamido-2,6-dideoxy-L-mannose), dTDP-N-acetylfucosamine, UDP-N-acetylfucosamine (UDP-L-FucNAc or UDP-2-acetamido-2,6-dideoxy-L-galactose), UDP-N-acetyl-L-pneumosamine (UDP-L-PneNAC or UDP-2-acetamido-2,6-dideoxy-L-talose), UDP-N-acetylmuramic acid, UDP-N-acetyl-L-quinovosamine (UDP-L-QuiNAc or UDP-2-acetamido-2,6-dideoxy-L-glucose), CMP-sialic acid (CMP-Neu5Ac), GDP-fucose (GDP-Fuc), GDP-rhamnose and UDP-xylose. In a more preferred embodiment of the method and/or cell of the invention, the cell is capable to synthesize at least the nucleotide-activated sugar CMP-Neu5Ac. In an even more preferred embodiment of the method and/or cell of the invention, the cell uses at least one of the synthesized nucleotide-activated sugars in the production of a sialylated oligosaccharide, preferably a 6′sialylated oligosaccharide, more preferably LSTc. The cell used herein is optionally genetically engineered to express the de novo synthesis of UDP-GlcNAc. UDP-GlcNAc can be provided by an enzyme expressed in the cell or by the metabolism of the cell. Such cell producing a UDP-GlcNAc can express enzymes converting, e.g. GlcNAc, which is to be added to the cell, to UDP-GlcNAc. These enzymes may be any one or more of the list comprising an N-acetyl-D-glucosamine kinase, an N-acetylglucosamine-6-phosphate deacetylase, a phosphoglucosamine mutase, and an N-acetylglucosamine-1-phosphate uridyltransferase/glucosamine-1-phosphate acetyltransferase from several species including Homo sapiens, Escherichia coli. Preferably, the cell is modified to produce UDP-GlcNAc. More preferably, the cell is modified for enhanced UDP-GlcNAc production. Said modification can be any one or more chosen from the group comprising knock-out of an N-acetylglucosamine-6-phosphate deacetylase, over-expression of an L-glutamine-D-fructose-6-phosphate aminotransferase, over-expression of a phosphoglucosamine mutase, and over-expression of an N-acetylglucosamine-1-phosphate uridyltransferase/glucosamine-1-phosphate acetyltransferase.

Additionally, or alternatively, the cell used herein is optionally genetically engineered to express the de novo synthesis of CMP-Neu5Ac. CMP-Neu5Ac can be provided by an enzyme expressed in the cell or by the metabolism of the cell. Such cell producing CMP-Neu5Ac can express an enzyme converting, e.g. sialic acid to CMP-Neu5Ac. This enzyme may be a CMP-sialic acid synthetase, like the N-acylneuraminate cytidylyltransferase from several species including Homo sapiens, Neisseria meningitidis, and Pasteurella multocida. Preferably, the cell is modified to produce CMP-Neu5Ac. More preferably, the cell is modified for enhanced CMP-Neu5Ac production. Said modification can be any one or more chosen from the group comprising knock-out of an N-acetylglucosamine-6-phosphate deacetylase, knock-out of a glucosamine-6-phosphate deaminase, over-expression of a CMP-sialic acid synthetase, and over-expression of an N-acetyl-D-glucosamine-2-epimerase encoding gene.

Additionally, or alternatively, the cell used herein is optionally genetically engineered to express the de novo synthesis of GDP-fucose. GDP-fucose can be provided by an enzyme expressed in the cell or by the metabolism of the cell. Such cell producing GDP-fucose can express an enzyme converting, e.g. fucose, which is to be added to the cell, to GDP-fucose. This enzyme may be, e.g. a bifunctional fucose kinase/fucose-1-phosphate guanylyltransferase, like Fkp from Bacteroides fragilis, or the combination of one separate fucose kinase together with one separate fucose-1-phosphate guanylyltransferase like they are known from several species including Homo sapiens, Sus scrofa and Rattus norvegicus. Preferably, the cell is modified to produce GDP-fucose. More preferably, the cell is modified for enhanced GDP-fucose production. Said modification can be any one or more chosen from the group comprising knock-out of a UDP-glucose:undecaprenyl-phosphate glucose-1-phosphate transferase encoding gene, over-expression of a GDP-L-fucose synthase encoding gene, over-expression of a GDP-mannose 4,6-dehydratase encoding gene, over-expression of a mannose-1-phosphate guanylyltransferase encoding gene, over-expression of a phosphomannomutase encoding gene and over-expression of a mannose-6-phosphate isomerase encoding gene.

Additionally, or alternatively, the cell used herein is optionally genetically engineered to express the de novo synthesis of UDP-Gal. UDP-Gal can be provided by an enzyme expressed in the cell or by the metabolism of the cell. Such cell producing UDP-Gal can express an enzyme converting, e.g. UDP-glucose, to UDP-Gal. This enzyme may be, e.g. the UDP-glucose-4-epimerase GalE like as known from several species including Homo sapiens, Escherichia coli, and Rattus norvegicus. Preferably, the cell is modified to produce UDP-Gal. More preferably, the cell is modified for enhanced UDP-Gal production. Said modification can be any one or more chosen from the group comprising knock-out of a bifunctional 5′-nucleotidase/UDP-sugar hydrolase encoding gene, knock-out of a galactose-1-phosphate uridylyltransferase encoding gene and over-expression of a UDP-glucose-4-epimerase encoding gene.

Additionally, or alternatively, the cell used herein is optionally genetically engineered to express the de novo synthesis of UDP-GalNAc. UDP-GalNAc can be synthesized from UDP-GlcNAc by the action of a single-step reaction using a UDP-N-acetylglucosamine 4-epimerase like e.g. wbgU from Plesiomonas shigelloides, gne from Yersinia enterocolitica or wbpP from Pseudomonas aeruginosa serotype O6. Preferably, the cell is modified to produce UDP-GalNAc. More preferably, the cell is modified for enhanced UDP-GalNAc production.

Additionally, or alternatively, the cell used herein is optionally genetically engineered to express the de novo synthesis of UDP-ManNAc. UDP-ManNAc can be synthesized directly from UDP-GlcNAc via an epimerization reaction performed by a UDP-GlcNAc 2-epimerase (like e.g. cap5P from Staphylococcus aureus, RffE from E. coli, Cps19fK from S. pneumoniae, and RfbC from S. enterica). Preferably, the cell is modified to produce UDP-ManNAc. More preferably, the cell is modified for enhanced UDP-ManNAc production.

According to another embodiment of the method and/or cell of the invention, the cell expresses at least one further glycosyltransferase chosen from the list comprising fucosyltransferases, sialyltransferases, galactosyltransferases, glucosyltransferases, mannosyltransferases, N-acetylglucosaminyltransferases, N-acetylgalactosaminyltransferases, N-acetylmannosaminyltransferases, xylosyltransferases, glucuronyltransferases, galacturonyltransferases, glucosaminyltransferases, N-glycolylneuraminyltransferases, rhamnosyltransferases, N-acetylrhamnosyltransferases, UDP-4-amino-4,6-dideoxy-N-acetyl-beta-L-altrosamine transaminases, UDP-N-acetylglucosamine enolpyruvyl transferases and fucosaminyltransferases.

In a preferred embodiment of the method and/or cell of the invention, the fucosyltransferase is chosen from the list comprising alpha-1,2-fucosyltransferase, alpha-1,3-fucosyltransferase, alpha-1,3/4-fucosyltransferase, alpha-1,4-fucosyltransferase and alpha-1,6-fucosyltransferase.

In an alternative and/or additional embodiment of the method and/or cell of the invention, the further sialyltransferase is chosen from the list comprising alpha-2,3-sialyltransferase, alpha-2,6-sialyltransferase, and alpha-2,8-sialyltransferase.

In an alternative and/or additional embodiment of the method and/or cell of the invention, the galactosyltransferase is chosen from the list comprising beta-1,3-galactosyltransferase, N-acetylglucosamine beta-1,3-galactosyltransferase, beta-1,4-galactosyltransferase, N-acetylglucosamine beta-1,4-galactosyltransferase, alpha-1,3-galactosyltransferase and alpha-1,4-galactosyltransferase.

In an alternative and/or additional embodiment of the method and/or cell of the invention, the glucosyltransferase is chosen from the list comprising alpha-glucosyltransferase, beta-1,2-glucosyltransferase, beta-1,3-glucosyltransferase and beta-1,4-glucosyltransferase.

In an alternative and/or additional embodiment of the method and/or cell of the invention, the mannosyltransferase is chosen from the list comprising alpha-1,2-mannosyltransferase, alpha-1,3-mannosyltransferase and alpha-1,6-mannosyltransferase.

In an alternative and/or additional embodiment of the method and/or cell of the invention, the N-acetylglucosaminyltransferase is chosen from the list comprising galactoside beta-1,3-N-acetylglucosaminyltransferase and beta-1,6-N-acetylglucosaminyltransferase.

In an alternative and/or additional embodiment of the method and/or cell of the invention, the N-acetylgalactosaminyltransferase is chosen from the list comprising alpha-1,3-N-acetylgalactosaminyltransferase.

In a further embodiment of the method and/or cell of the invention, the cell is modified in the expression or activity of at least one of said glycosyltransferases. In a preferred embodiment, said glycosyltransferase is an endogenous protein of the cell with a modified expression or activity, preferably said endogenous glycosyltransferase is overexpressed; alternatively said glycosyltransferase is a heterologous protein that is heterogeneously introduced and expressed in said cell, preferably overexpressed. Said endogenous glycosyltransferase can have a modified expression in the cell which also expresses a heterologous glycosyltransferase.

According to another and/or alternative preferred embodiment of the method and/or cell of the invention, the cell comprises a fucosylation pathway comprising at least one enzyme chosen from the list comprising mannose-6-phosphate isomerase, phosphomannomutase, mannose-1-phosphate guanylyltransferase, GDP-mannose 4,6-dehydratase, GDP-L-fucose synthase, fucose permease, fucose kinase, fucose-1-phosphate guanylyltransferase, fucosyltransferase.

According to another and/or alternative preferred embodiment of the method and/or cell of the invention, the cell comprises a galactosylation pathway comprising at least one enzyme chosen from the list comprising galactose-1-epimerase, galactokinase, glucokinase, galactose-1-phosphate uridylyltransferase, UDP-glucose 4-epimerase, glucose-1-phosphate uridylyltransferase, glucophosphomutase, galactosyltransferase.

According to another and/or alternative preferred embodiment of the method and/or cell of the invention, the cell comprises an N-acetylglucosaminylation pathway comprising at least one enzyme chosen from the list comprising L-glutamine-D-fructose-6-phosphate aminotransferase, N-acetylglucosamine-6-phosphate deacetylase, phosphoglucosamine mutase, N-acetylglucosamine-1-phosphate uridylyltransferase/glucosamine-1-phosphate acetyltransferase, N-acetylglucosaminyltransferase.

In an alternative and/or additional further embodiment of the method and/or cell of the invention, the cell is modified in the expression or activity of at least one pyruvate dehydrogenase like e.g. from E. coli, S. cerevisiae, H. sapiens and R. norvegicus. In a preferred embodiment, the cell has been modified to have at least one partially or fully knocked out or mutated pyruvate dehydrogenase encoding gene by means generally known by the person skilled in the art resulting in at least one protein with less functional or being disabled for pyruvate dehydrogenase activity. In a more preferred embodiment, the cell has a full knock-out in the poxB encoding gene resulting in a cell lacking pyruvate dehydrogenase activity.

In an alternative and/or additional further embodiment of the method and/or cell of the invention, the cell is modified in the expression or activity of at least one lactate dehydrogenase like e.g. from E. coli, S. cerevisiae, H. sapiens and R. norvegicus. In a preferred embodiment, the cell has been modified to have at least one partially or fully knocked out or mutated lactate dehydrogenase encoding gene by means generally known by the person skilled in the art resulting in at least one protein with less functional or being disabled for lactate dehydrogenase activity. In a more preferred embodiment, the cell has a full knock-out in the ldhA encoding gene resulting in a cell lacking lactate dehydrogenase activity.

According to another preferred embodiment of the method and/or cell of the invention, the cell comprises a lower or reduced expression and/or abolished, impaired, reduced or delayed activity of any one or more of the proteins comprising beta-galactosidase, galactoside O-acetyltransferase, N-acetylglucosamine-6-phosphate deacetylase, glucosamine-6-phosphate deaminase, N-acetylglucosamine repressor, ribonucleotide monophosphatase, EIICBA-Nag, UDP-glucose:undecaprenyl-phosphate glucose-1-phosphate transferase, L-fuculokinase, L-fucose isomerase, N-acetylneuraminate lyase, N-acetylmannosamine kinase, N-acetylmannosamine-6-phosphate 2-epimerase, EIIAB-Man, EIIC-Man, EIID-Man, ushA, galactose-1-phosphate uridylyltransferase, glucose-1-phosphate adenylyltransferase, glucose-1-phosphatase, ATP-dependent 6-phosphofructokinase isozyme 1, ATP-dependent 6-phosphofructokinase isozyme 2, glucose-6-phosphate isomerase, aerobic respiration control protein, transcriptional repressor IcIR, Ion protease, glucose-specific translocating phosphotransferase enzyme IIBC component ptsG, glucose-specific translocating phosphotransferase (PTS) enzyme IIBC component malX, enzyme IIA^{Glc}, beta-glucoside specific PTS enzyme II, fructose-specific PTS multiphosphoryl transfer protein FruA and FruB, ethanol dehydrogenase aldehyde dehydrogenase, pyruvate-formate lyase, acetate kinase, phosphoacyltransferase, phosphate acetyltransferase, pyruvate decarboxylase.

According to another preferred embodiment of the method and/or cell of the invention, the cell is using a precursor for the synthesis of a sialylated oligosaccharide, preferably a 6′sialylated oligosaccharide, more preferably LSTc. Herein, the precursor is fed to the cell from the cultivation or incubation medium. In another preferred embodiment, the cell is producing a precursor for the synthesis of said sialylated oligosaccharide.

According to another preferred embodiment of the method and/or cell of the invention, the cell produces 90 g/L or more of a sialylated oligosaccharide, preferably a 6′sialylated oligosaccharide, more preferably LSTc, in the whole broth and/or supernatant. In a more preferred embodiment, a sialylated oligosaccharide, preferably a 6′sialylated oligosaccharide, more preferably LSTc, produced in the whole broth and/or supernatant has a purity of at least 80% measured on the total amount of the sialylated oligosaccharide, preferably a 6′sialylated oligosaccharide, more preferably LSTc, and its precursor produced by the cell in the whole broth and/or supernatant, respectively.

According to another embodiment of the method and/or cell of the invention, the sialylated oligosaccharide is chosen from the list comprising a milk oligosaccharide, 0-antigen, the oligosaccharide repeats present in capsular polysaccharides and amino-sugars. In a more preferred embodiment, the milk oligosaccharide is a mammalian milk oligosaccharide. In an even more preferred embodiment, the milk oligosaccharide is a human milk oligosaccharide.

According to another embodiment of the method and/or cell of the invention, the cell is capable to synthesize a mixture of oligosaccharides. In an alternative and/or additional embodiment, the cell is capable to synthesize a mixture of di- and oligosaccharides, alternatively, the cell is capable to synthesize a mixture of sialic acid, di- and/or oligosaccharides.

Another aspect of the invention provides for a method and a cell wherein a sialylated oligosaccharide, preferably a 6′sialylated oligosaccharide, more preferably LSTc, is produced in and/or by a cell which is a bacterium, fungus, yeast, a plant cell, an animal cell, or a protozoan cell. Preferably said bacterium is an Escherichia coli strain, more preferably an Escherichia coli strain which is a K-12 strain, even more preferably the Escherichia coli K-12 strain is E. coli MG1655. Preferably said fungus belongs to a genus chosen from the group comprising Rhizopus, Dictyostelium, Penicillium, Mucor or Aspergillus. The latter yeast preferably belongs to the phylum of the Ascomycota or the phylum of the Basidiomycota or the phylum of the Deuteromycota or the phylum of the Zygomycetes. Preferably said yeast belongs to a genus chosen from the group comprising Saccharomyces (with members like e.g. Saccharomyces cerevisiae, S. bayanus, S. boulardii), Zygosaccharomyces, Pichia (with members like e.g. Pichia pastoris, P. anomala, P. kluyveri), Komagataella, Hansenula, Candida, Schizosaccharomyces, Schwanniomyces, Torulaspora, Yarrowia like e.g. Yarrowia lipolytica), Starmerella like e.g. Starmerella bombicola), Kluyveromyces (with members like e.g. Kluyveromyces lactis, K. marxianus, K. thermotolerans) or Debaromyces. The latter yeast is preferably selected from Pichia pastoris, Yarrowia lipolitica, Saccharomyces cerevisiae, Kluyveromyces lactis, Hansenula polymorpha, Kluyveromyces marxianus, Pichia methanolica, Pichia stipites, Candida boidinii, Schizosaccharomyces pombe, Schwanniomyces occidentalis, Torulaspora delbrueckii, Zygosaccharomyces rouxii, and Zygosaccharomyces bailii. Plant cells include cells of flowering and non-flowering plants, as well as algal cells, for example Chlamydomonas, Chlorella, etc. Preferably, said plant cell is an algal cell or is derived from tobacco, alfalfa, rice, tomato, cotton, rapeseed, soy, maize, or corn plant. Preferably said animal cell is derived from non-human mammals (e.g. cattle, buffalo, pig, sheep, mouse, rat, primate (e.g. chimpanzee, orangutan, gorilla, monkey (e.g. Old World, New World), lemur), dog, cat, rabbit, horse, cow, goat, ox, deer, musk deer, bovid, whale, dolphin, hippopotamus, elephant, rhinoceros, giraffe, zebra, lion, cheetah, tiger, panda, red panda, otter), birds (e.g. chicken, duck, ostrich, turkey, pheasant), fish (e.g. swordfish, salmon, tuna, sea bass, trout, catfish), invertebrates (e.g. lobster, crab, shrimp, clams, oyster, mussel, sea urchin), reptiles (e.g. snake, alligator, turtle), amphibians (e.g. frogs) or insects (e.g. fly, nematode) or is a genetically modified or engineered cell line derived from human cells excluding embryonic stem cells. Both human and non-human mammalian cells are preferably chosen from the list comprising an epithelial cell like e.g. a mammary epithelial cell, an embryonic kidney cell (e.g. HEK293 or HEK 293T cell), a fibroblast cell, a COS cell, a Chinese hamster ovary (CHO) cell, a murine myeloma cell like e.g. an N20, SP2/0 or YB2/0 cell, an NIH-3T3 cell, a non-mammary adult stem cell or derivatives thereof such as described in WO21067641, a lactocyte derived from mammalian induced pluripotent stem cells, preferably human induced pluripotent stem cells, a lactocyte as part of mammary-like gland organoids, a post-parturition mammary epithelium cell, a polarized mammary cell, preferably a polarized mammary cell selected from the group comprising live primary mammary epithelial cells, live mammary myoepithelial cells, live mammary progenitor cells, live immortalized mammary epithelial cells, live immortalized mammary myoepithelial cells, live immortalized mammary progenitor cells, a non-mammary adult stem cell or derivatives thereof as well-known to the person skilled in the art from e.g. WO 2021/219634, WO 2022/054053, WO 2021/141762, WO 2021/142241, WO 2021/067641 and WO 2021/242866. More preferably said insect cell is derived from Spodoptera frugiperda like e.g. Sf9 or Sf21 cells, Bombyx mori, Mamestra brassicae, Trichoplusia ni like e.g. BTI-TN-5B1-4 cells or Drosophila melanogaster like e.g. Drosophila S2 cells. Preferably, said protozoan cell is a Leishmania tarentolae cell. More preferably, the cell is selected from the group consisting of prokaryotic cells and eukaryotic cells, preferably from the group consisting of yeast cells, bacterial cells, archaebacterial cells, algae cells, and fungal cells as described herein.

According to another embodiment of the method and/or cell of the invention, the cell as described herein comprises a nucleic acid molecule comprising a polynucleotide sequence encoding an alpha-2,6-sialyltransferase as described herein and operably linked to control sequences recognized by the cell, wherein said sequence is foreign to the cell, said sequence further i) being integrated in the genome of said cell and/or ii) presented to said cell on a vector.

According to another preferred embodiment of the method and/or cell of the invention, the cell comprises a catabolic pathway for selected mono-, di- or oligosaccharides which is at least partially inactivated, the mono-, di-, or oligosaccharides being involved in and/or required for the synthesis of a sialylated oligosaccharide. Preferably, said sialylated oligosaccharide is a 6′sialylated oligosaccharide, more preferably LSTc.

A further aspect of the present invention provides for an isolated nucleic acid molecule encoding an alpha-2,6-sialyltransferase as described herein.

Another aspect of the present invention provides for a vector comprising an isolated nucleic acid molecule encoding an alpha-2,6-sialyltransferase as described herein.

Another aspect provides for a cell to be stably cultured in a medium, wherein said medium can be any type of growth medium comprising minimal medium, complex medium or growth medium enriched in certain compounds like for example but not limited to vitamins, trace elements, amino acids.

The microorganism or cell as used herein is capable to grow on a monosaccharide, disaccharide, oligosaccharide, polysaccharide, polyol, glycerol, a complex medium or a mixture thereof as the main carbon source. With the term main is meant the most important carbon source for the microorganism or cell for the production of the sialylated oligosaccharide of interest, biomass formation, carbon dioxide and/or by-products formation (such as acids and/or alcohols, such as acetate, lactate, and/or ethanol), i.e. 20, 30, 40, 50, 60, 70, 75, 80, 85, 90, 95, 98, 99% of all the required carbon is derived from the above-indicated carbon source. In one embodiment of the invention, said carbon source is the sole carbon source for said organism, i.e. 100% of all the required carbon is derived from the above-indicated carbon source.

Common main carbon sources comprise but are not limited to glucose, glycerol, fructose, sucrose, maltose, lactose, arabinose, malto-oligosaccharides, maltotriose, sorbitol, xylose, rhamnose, galactose, mannose, methanol, ethanol, trehalose, starch, cellulose, hemi-cellulose, molasses, corn-steep liquor, high-fructose syrup, acetate, citrate, lactate and pyruvate. As used herein, a precursor as defined herein cannot be used as a carbon source for the production of the sialylated oligosaccharide of present invention.

According to the present invention, the methods as described herein preferably comprises a step of separating the sialylated oligosaccharide of present invention from said cultivation or incubation, otherwise said recovering the sialylated oligosaccharide from the cultivation or incubation medium and/or the cell.

The terms “separating from said cultivation or incubation” means harvesting, collecting, or retrieving said sialylated oligosaccharide from the cell and/or the medium of its cultivation or incubation.

The sialylated oligosaccharide can be separated in a conventional manner from the aqueous culture medium, in which the cell was cultivated or incubated. In case said sialylated oligosaccharide is still present in the cells producing the sialylated oligosaccharide, conventional manners to free or to extract said sialylated oligosaccharide out of the cells can be used, such as cell destruction using high pH, heat shock, sonication, French press, homogenization, enzymatic hydrolysis, chemical hydrolysis, solvent hydrolysis, detergent, hydrolysis, etc. The cultivation or incubation medium and/or cell extract together and separately can then be further used for separating said sialylated oligosaccharide.

This preferably involves clarifying said sialylated oligosaccharide to remove suspended particulates and contaminants, particularly cells, cell components, insoluble metabolites and debris produced by culturing or incubating the genetically engineered cell. In this step, said sialylated oligosaccharide can be clarified in a conventional manner. Preferably, said sialylated oligosaccharide is clarified by centrifugation, flocculation, decantation and/or filtration. Another step of separating said sialylated oligosaccharide preferably involves removing substantially all the eventually remaining proteins, peptides, amino acids, RNA and DNA, and any endotoxins and glycolipids that could interfere with the subsequent separation step, from said sialylated oligosaccharide, preferably after it has been clarified. In this step, remaining proteins and related impurities can be removed from said sialylated oligosaccharide in a conventional manner. Preferably, remaining proteins, salts, by-products, colour, endotoxins and other related impurities are removed from said sialylated oligosaccharide by ultrafiltration, nanofiltration, two-phase partitioning, reverse osmosis, microfiltration, activated charcoal or carbon treatment, treatment with non-ionic surfactants, enzymatic digestion, tangential flow high-performance filtration, tangential flow ultrafiltration, electrophoresis (e.g. using slab-polyacrylamide or sodium dodecyl sulphate-polyacrylamide gel electrophoresis (PAGE)), affinity chromatography (using affinity ligands including e.g. DEAE-Sepharose, poly-L-lysine and polymyxin-B, endotoxin-selective adsorber matrices), ion exchange chromatography (such as but not limited to cation exchange, anion exchange, mixed bed ion exchange, inside-out ligand attachment), hydrophobic interaction chromatography and/or gel filtration (i.e. size exclusion chromatography), particularly by chromatography, more particularly by ion exchange chromatography or hydrophobic interaction chromatography or ligand exchange chromatography or electrodialysis. With the exception of size exclusion chromatography, remaining proteins and related impurities are retained by a chromatography medium or a selected membrane.

In a further preferred embodiment, the methods as described herein also provide for a further purification of the sialylated oligosaccharide of present invention. A further purification of said sialylated oligosaccharide may be accomplished, for example, by use of (activated) charcoal or carbon, nanofiltration, ultrafiltration, electrophoresis, enzymatic treatment or ion exchange, temperature adjustment, pH adjustment or pH adjustment with an alkaline or acidic solution to remove any remaining DNA, protein, LPS, endotoxins, or other impurity. Alcohols, such as ethanol, and aqueous alcohol mixtures can also be used. Another purification step is accomplished by crystallization, evaporation or precipitation of said sialylated oligosaccharide. Another purification step is to dry, e.g. spray dry, lyophilize, spray freeze dry, freeze spray dry, band dry, belt dry, vacuum band dry, vacuum belt dry, drum dry, roller dry, vacuum drum dry or vacuum roller dry the produced sialylated oligosaccharide.

In an exemplary embodiment, the separation and purification of the sialylated oligosaccharide is made in a process, comprising the following steps in any order:
a) contacting the cultivation or incubation or a clarified version thereof with a nanofiltration membrane with a molecular weight cut-off (MWCO) of 600-3500 Da ensuring the retention of the produced sialylated oligosaccharide and allowing at least a part of the proteins, salts, by-products, colour and other related impurities to pass,
b) conducting a diafiltration process on the retentate from step a), using said membrane, with an aqueous solution of an inorganic electrolyte, followed by optional diafiltration with pure water to remove excess of the electrolyte,
c) and collecting the retentate enriched in said sialylated oligosaccharide in the form of a salt from the cation of said electrolyte.

In an alternative exemplary embodiment, the separation and purification of said sialylated oligosaccharide is made in a process, comprising the following steps in any order: subjecting the cultivation or incubation or a clarified version thereof to two membrane filtration steps using different membranes, wherein
one membrane has a molecular weight cut-off of between about 300 to about 500 Dalton, and
the other membrane as a molecular weight cut-off of between about 600 to about 800 Dalton.

In an alternative exemplary embodiment, the separation and purification of said sialylated oligosaccharide is made in a process, comprising treating the cultivation or incubation or a clarified version thereof with a strong cation exchange resin in H+-form in a step and a weak anion exchange resin in free base form, in another step, wherein said steps can be performed in any order.

In an alternative exemplary embodiment, the separation and purification of said sialylated oligosaccharide is made in the following way. The cultivation or incubation comprising the produced sialylated oligosaccharide, biomass, medium components and contaminants is applied to the following purification steps:
i) separation of biomass from the cultivation or incubation,
ii) cationic ion exchanger treatment for the removal of positively charged material,
iii) anionic ion exchanger treatment for the removal of negatively charged material,
iv) nanofiltration step and/or electrodialysis step,

wherein a purified solution comprising the produced sialylated oligosaccharide at a purity of greater than or equal to 80% is provided. Optionally the purified solution is dried by any one or more drying steps chosen from the list comprising spray drying, lyophilization, spray freeze drying, freeze spray drying, band drying, belt drying, vacuum band drying, vacuum belt drying, drum drying, roller drying, vacuum drum drying and vacuum roller drying.

In an alternative exemplary embodiment, the separation and purification of the sialylated oligosaccharide is made in a process, comprising the following steps in any order: enzymatic treatment of the cultivation or incubation; removal of the biomass from the cultivation or incubation; ultrafiltration; nanofiltration; and a column chromatography step. Preferably such column chromatography is a single column or a multiple column. Further preferably the column chromatography step is simulated moving bed chromatography. Such simulated moving bed chromatography preferably comprises i) at least 4 columns, wherein at least one column comprises a weak or strong cation exchange resin; and/or ii) four zones I, II, 11 and IV with different flow rates; and/or iii) an eluent comprising water; and/or iv) an operating temperature of 15 degrees to 60 degrees centigrade.

In a specific embodiment, the present invention provides the produced sialylated oligosaccharide which is dried to powder by any one or more drying steps chosen from the list comprising spray drying, lyophilization, spray freeze drying, freeze spray drying, band drying, belt drying, vacuum band drying, vacuum belt drying, drum drying, roller drying, vacuum drum drying and vacuum roller drying, wherein the dried powder contains <15%-wt. of water, preferably <10%-wt. of water, more preferably <7%-wt. of water, most preferably <5%-wt. of water.

Another aspect of the present invention provides the use of an alpha-2,6-sialyltransferase as described herein for production of a sialylated oligosaccharide, preferably a 6′sialylated oligosaccharide, more preferably LSTc.

A further aspect of the present invention provides the use of a cell as described herein for production of a sialylated oligosaccharide, preferably a 6′sialylated oligosaccharide, more preferably LSTc.

Still a further aspect of the present invention provides i) use of a method as described herein for production of a sialylated oligosaccharide, preferably a 6′sialylated oligosaccharide, more preferably LSTc, ii) use of an isolated nucleic acid molecule as described herein for production of a sialylated oligosaccharide, preferably a 6′sialylated oligosaccharide, more preferably LSTc, or iii) use of a vector as described herein for production of a sialylated oligosaccharide, preferably a 6′sialylated oligosaccharide, more preferably LSTc.

Furthermore, the invention also relates to the sialylated oligosaccharide obtained by the methods according to the invention Said sialylated oligosaccharide may be used for the manufacture of a preparation, as food additive, prebiotic, symbiotic, for the supplementation of baby food, adult food, infant animal feed, adult animal feed, or as either therapeutically or pharmaceutically active compound or in cosmetic applications. In a preferred embodiment, said preparation comprises at least one sialylated oligosaccharide, preferably a 6′sialylated oligosaccharide, more preferably LSTc, that is obtainable, preferably obtained, by the methods as described herein. In another preferred embodiment, a preparation is provided that further comprises at least one probiotic microorganism. In another preferred embodiment of present invention, said preparation is a nutritional composition. In a more preferred embodiment, said preparation is a medicinal formulation, a dietary supplement, a dairy drink or an infant formula.

With the novel methods, the sialylated oligosaccharide can easily and effectively be provided, without the need for complicated, time and cost consuming synthetic processes.

For identification of the sialylated oligosaccharide of present invention produced as described herein, the monosaccharide or the monomeric building blocks (e.g. the monosaccharide or glycan unit composition), the anomeric configuration of side chains, the presence and location of substituent groups, degree of polymerization/molecular weight and the linkage pattern can be identified by standard methods known in the art, such as, e.g. methylation analysis, reductive cleavage, hydrolysis, GC-MS (gas chromatography-mass spectrometry), MALDI-MS (Matrix-assisted laser desorption/ionization-mass spectrometry), ESI-MS (Electrospray ionization-mass spectrometry), HPLC (High-Performance Liquid chromatography with ultraviolet or refractive index detection), HPAEC-PAD (High-Performance Anion-Exchange chromatography with Pulsed Amperometric Detection), CE (capillary electrophoresis), IR (infrared)/Raman spectroscopy, and NMR (Nuclear magnetic resonance) spectroscopy techniques. The crystal structure can be solved using, e.g. solid-state NMR, FT-IR (Fourier transform infrared spectroscopy), and WAXS (wide-angle X-ray scattering). The degree of polymerization (DP), the DP distribution, and polydispersity can be determined by, e.g. viscosimetry and SEC (SEC-HPLC, high performance size-exclusion chromatography). To identify the monomeric components of the sialylated oligosaccharide methods such as e.g. acid-catalysed hydrolysis, HPLC (high performance liquid chromatography) or GLC (gas-liquid chromatography) (after conversion to alditol acetates) may be used. To determine the glycosidic linkages, the sialylated oligosaccharide is methylated with methyl iodide and strong base in DMSO, hydrolysis is performed, a reduction to partially methylated alditols is achieved, an acetylation to methylated alditol acetates is performed, and the analysis is carried out by GLC/MS (gas-liquid chromatography coupled with mass spectrometry). To determine the glycan sequence, a partial depolymerization is carried out using an acid or enzymes to determine the structures. To identify the anomeric configuration, the sialylated oligosaccharide is subjected to enzymatic analysis, e.g. it is contacted with an enzyme that is specific for a particular type of linkage, e.g. beta-galactosidase, or alpha-glucosidase, etc., and NMR may be used to analyse the products.

The separated and preferably also purified sialylated oligosaccharide as described herein is incorporated into a food (e.g. human food or feed), dietary supplement, pharmaceutical ingredient, cosmetic ingredient or medicine. In some embodiments, the sialylated oligosaccharide is mixed with one or more ingredients suitable for food, feed, dietary supplement, pharmaceutical ingredient, cosmetic ingredient or medicine.

In some embodiments, the dietary supplement comprises at least one prebiotic ingredient and/or at least one probiotic ingredient.

A “prebiotic” is a substance that promotes growth of microorganisms beneficial to the host, particularly microorganisms in the gastrointestinal tract. In some embodiments, a dietary supplement provides multiple prebiotics, including the 6′sialylated oligosaccharide being a prebiotic produced and/or purified by a process disclosed in this specification, to promote growth of one or more beneficial microorganisms.

Examples of prebiotic ingredients for dietary supplements include other prebiotic molecules (such as HMOs) and plant polysaccharides (such as inulin, pectin, b-glucan and xylooligosaccharide). A “probiotic” product typically contains live microorganisms that replace or add to gastrointestinal microflora, to the benefit of the recipient. Examples of such microorganisms include Lactobacillus species (for example, L. acidophilus and L. bulgaricus), Bifidobacterium species (for example, B. animalis, B. longum and B. infantis (e.g. Bi-26)), and Saccharomyces boulardii. In some embodiments, a sialylated oligosaccharide produced and/or purified by a process of this specification is orally administered in combination with such microorganism.

Examples of further ingredients for dietary supplements include oligosaccharides (such as 2′-fucosyllactose, 3-fucosyllactose, 3′-sialyllactose, 6′-sialyllactose), disaccharides (such as lactose), monosaccharides (such as glucose, galactose, L-fucose, sialic acid, glucosamine and N-acetylglucosamine), thickeners (such as gum arabic), acidity regulators (such as trisodium citrate), water, skimmed milk, and flavourings.

In some embodiments, the sialylated oligosaccharide is incorporated into a human baby food (e.g. infant formula). Infant formula is generally a manufactured food for feeding to infants as a complete or partial substitute for human breast milk. In some embodiments, infant formula is sold as a powder and prepared for bottle- or cup-feeding to an infant by mixing with water. The composition of infant formula is typically designed to be roughly mimic human breast milk. In some embodiments, a sialylated oligosaccharide produced and/or purified by a process in this specification is included in infant formula to provide nutritional benefits similar to those provided by the oligosaccharides in human breast milk. In some embodiments, the sialylated oligosaccharide is mixed with one or more ingredients of the infant formula.

Examples of infant formula ingredients include non-fat milk, carbohydrate sources (e.g. lactose), protein sources (e.g. whey protein concentrate and casein), fat sources (e.g. vegetable oils—such as palm, high oleic safflower oil, rapeseed, coconut and/or sunflower oil; and fish oils), vitamins (such as vitamins A, Bb, Bi2, C and D), minerals (such as potassium citrate, calcium citrate, magnesium chloride, sodium chloride, sodium citrate and calcium phosphate) and possibly human milk oligosaccharides (HMOs). Such HMOs may include, for example, DiFL, lacto-N-triose II, LNT, LNnT, lacto-N-fucopentaose I, lacto-N-neofucopentaose, lacto-N-fucopentaose II, lacto-N-fucopentaose Ill, lacto-N-fucopentaose V, lacto-N-neofucopentaose V, lacto-N-difucohexaose I, lacto-N-difucohexaose II, 6′-galactosyllactose, 3′-galactosyllactose, lacto-N-hexaose and lacto-N-neohexaose.

In some embodiments, the one or more infant formula ingredients comprise non-fat milk, a carbohydrate source, a protein source, a fat source, and/or a vitamin and mineral.

In some embodiments, the one or more infant formula ingredients comprise lactose, whey protein concentrate and/or high oleic safflower oil.

In some embodiments, the concentration of the sialylated oligosaccharide in the infant formula is approximately the same concentration as the concentration of the sialylated oligosaccharide generally present in human breast milk.

In some embodiments, the sialylated oligosaccharide is incorporated into a feed preparation, wherein said feed is chosen from the list comprising pet food, animal milk replacer, veterinary product, veterinary feed supplement, nutrition supplement, post weaning feed, or creep feed.

As will be shown in the examples herein, the newly identified alpha-2,6-sialyltransferase have proven to be useful in the enzymatic and cell-based production of sialylated oligosaccharides, preferably a 6′sialylated oligosaccharide, more preferably LSTc. The methods and the cell of the invention preferably provide at least one of the following further surprising advantages when using the alpha-2,6-sialyltransferase as defined herein:
Higher preference for LNnT than lactose as an acceptor for sialylation,
Higher titres of the sialylated oligosaccharide (g/L),
Higher production rate r (g sialylated oligosaccharide/L/h),
Higher cell performance index CPI (g sialylated oligosaccharide/g X),
Higher specific productivity Qp (g sialylated oligosaccharide/g X/h),
Higher yield on sucrose Ys (g sialylated oligosaccharide/g sucrose),
Higher sucrose uptake/conversion rate Qs (g sucrose/g X/h),
Higher lactose conversion/consumption rate rs (g lactose/h),
Higher secretion of the sialylated oligosaccharide, and/or
Higher growth speed of the production host,

when compared to a method or a cell using an identical setup or enzymatic or genetic background but lacking the use of the newly identified alpha-2,6-sialyltransferase.

Unless defined otherwise, all technical and scientific terms used herein generally have the same meaning as commonly understood by one of ordinary skill in the art to which this invention belongs. Generally, the nomenclature used herein and the laboratory procedures in cell culture, molecular genetics, organic chemistry and nucleic acid chemistry and hybridization described above and below are those well-known and commonly employed in the art. Standard techniques are used for nucleic acid and peptide synthesis. Generally, purification steps are performed according to the manufacturer's specifications.

Further advantages follow from the specific embodiments and the examples. It goes without saying that the abovementioned features and the features which are still to be explained below can be used not only in the respectively specified combinations, but also in other combinations or on their own, without departing from the scope of the present invention.

Moreover, the present invention relates to the following specific embodiments:
1. An alpha-2,6-sialyltransferase having alpha-2,6-sialyltransferase activity on the terminal galactose (Gal) residue of lacto-N-neotetraose (LNnT, Gal-β1,4-GlcNAc-β1,3-Gal-β1,4-Glc) and comprising an amino acid sequence that is at least 60.0% identical over a stretch of at least 100 amino acid residues, preferably at least 150 amino acid residues, more preferably at least 200 amino acid residues, to the amino acid sequence as represented by SEQ ID NO 1.
2. An alpha-2,6-sialyltransferase according to embodiment 1, wherein said alpha-2,6-sialyltransferase comprises an amino acid sequence that is at least 60.0%, at least 65.0%, at least 70.0%, at least 75.0%, at least 80.0%, at least 85.0%, at least 90.0%, at least 95.0%, at least 96.0%, at least 97.0%, at least 98.0%, at least 98.5%, or at least 99.0% identical to the amino acid sequence as represented by SEQ ID NO 1 over a stretch of at least 100, at least 110, at least 120, at least 130, at least 140, at least 150, at least 160, at least 170, at least 180, at least 190, at least 200, at least 210, at least 220, at least 230, at least 240, at least 250, at least 260, at least 270, at least 280, at least 290 or at least 300 amino acid residues.
3. An alpha-2,6-sialyltransferase according to any one of embodiment 1 or 2, wherein said alpha-2,6-sialyltransferase comprises an amino acid sequence that is at least 60.0%, at least 65.0%, at least 70.0%, at least 75.0%, at least 80.0%, at least 85.0%, at least 90.0%, at least 95.0%, at least 96.0%, at least 97.0%, at least 98.0%, at least 98.5%, or at least 99.0% identical to the full-length amino acid sequence as represented by SEQ ID NO 1.
4. An alpha-2,6-sialyltransferase according to any one of embodiment 1 to 3, wherein said alpha-2,6-sialyltransferase comprises an amino acid sequence as represented by SEQ ID NO 1.
5. An alpha-2,6-sialyltransferase according to any one of embodiment 1 to 4 for use in the production of a sialylated oligosaccharide, preferably a 6′sialylated oligosaccharide, more preferably LSTc (Neu5Ac-α2,6-Gal-β1,4-GlcNAc-β1,3-Gal-β1,4-Glc).
6. Method for the production of a sialylated oligosaccharide, preferably a 6′sialylated oligosaccharide, more preferably LSTc (Neu5Ac-α2,6-Gal-β1,4-GlcNAc-β1,3-Gal-β1,4-Glc), the method comprising: contacting an alpha-2,6-sialyltransferase of any one of embodiments 1 to 5 with a mixture comprising a donor comprising a sialic acid residue, and an acceptor chosen from the list comprising an oligosaccharide or a disaccharide, under conditions where said alpha-2,6-sialyltransferase catalyses the transfer of a sialic acid residue from the donor to the acceptor, thereby producing said sialylated oligosaccharide.
7. Method according to embodiment 6, wherein said sialylated oligosaccharide is LSTc and said acceptor is lacto-N-neotetraose (LNnT, Gal-β1,4-GlcNAc-β1,3-Gal-β1,4-Glc).
8. Method for the production of a sialylated oligosaccharide, preferably a 6′sialylated oligosaccharide, more preferably LSTc (Neu5Ac-α2,6-Gal-β1,4-GlcNAc-β1,3-Gal-β1,4-Glc), said method comprising the steps of:
   a) providing
      i. CMP-sialic acid,
      ii. an acceptor, preferably lacto-N-neotetraose (LNnT, Gal-β1,4-GlcNAc-β1,3-Gal-β1,4-Glc), and
      iii. a sialyltransferase, wherein said sialyltransferase is an alpha-2,6-sialyltransferase of any one of embodiments 1 to 5,
   b) contacting said sialyltransferase and CMP-sialic acid with said acceptor, preferably LNnT, under conditions where the sialyltransferase catalyses the transfer of a sialic acid residue from said CMP-sialic acid to the acceptor resulting in the production of a sialylated oligosaccharide, preferably a 6′sialylated oligosaccharide, more preferably LSTc (Neu5Ac-α2,6-Gal-β1,4-GlcNAc-(β1,3-Gal-β1,4-Glc),
   c) preferably, separating said produced sialylated oligosaccharide.
9. Method according to any one of embodiments 6 to 8, the method comprising: contacting a cell extract comprising an alpha-2,6-sialyltransferase of any one of embodiments 1 to 5 with a mixture comprising a donor comprising a sialic acid residue, and an acceptor comprising an oligosaccharide or disaccharide, under conditions where said alpha-2,6-sialyltransferase catalyses the transfer of a sialic acid residue from the donor to the acceptor, thereby producing said sialylated oligosaccharide, preferably a 6′sialylated oligosaccharide, more preferably LSTc.
10. Method according to any one of embodiments 6 to 9, wherein said sialylated oligosaccharide, preferably a 6′sialylated oligosaccharide, more preferably LSTc, is produced in a cell-free system.
11. Method for the production of a sialylated oligosaccharide, preferably a 6′sialylated oligosaccharide, more preferably LSTc (Neu5Ac-α2,6-Gal-β1,4-GlcNAc-β1,3-Gal-β1,4-Glc), said method comprising the steps of:
   i. providing a cell, preferably a single cell, expressing, preferably heterologously expressing, more preferably overexpressing, even more preferably heterologously overexpressing, an alpha-2,6-sialyltransferase of any one of embodiments 1 to 5,
   ii. providing CMP-sialic acid, optionally said CMP-sialic acid is produced by said cell, and
   iii. providing an oligosaccharide or disaccharide, optionally said oligosaccharide or disaccharide is produced by said cell, and
   iv. cultivating and/or incubating said cell under conditions permissive to express said alpha-2,6-sialyltransferase, optionally permissive to produce said CMP-sialic acid and/or said oligosaccharide or disaccharide,
   v. preferably, separating said sialylated oligosaccharide, preferably 6′sialylated oligosaccharide, more preferably LSTc, from said cultivation or incubation.
12. Method according to embodiment 11, wherein said cell is a metabolically engineered cell, preferably wherein said cell is metabolically engineered for the production of said sialylated oligosaccharide, preferably 6′sialylated oligosaccharide, more preferably LSTc.
13. Method according to any one of embodiment 11 or 12, wherein the cultivation medium contains at least one carbon source selected from the group consisting of glucose, fructose, sucrose, and glycerol.
14. Method according to any one of embodiments 6 to 13, wherein the cultivation or incubation medium contains at least one compound selected from the group consisting of lactose, galactose, lacto-N-neotetraose (LNnT), UDP-galactose (UDP-Gal), UDP-N-acetylglucosamine (UDP-GlcNAc), sialic acid and CMP-sialic acid.
15. Method according to any one of embodiments 6 to 14, wherein said sialylated oligosaccharide, preferably 6′sialylated oligosaccharide, more preferably LSTc, is recovered from the cultivation or incubation medium and/or the cell.
16. Method according to any one of embodiments 6 to 15, the method comprising:
   i) Use of a cultivation or incubation medium comprising at least one precursor and/or acceptor for the production of said sialylated oligosaccharide, preferably 6′sialylated oligosaccharide, more preferably LSTc, and/or
   ii) Adding to the cultivation or incubation medium at least one precursor and/or acceptor feed for the production of said sialylated oligosaccharide, preferably 6′sialylated oligosaccharide, more preferably LSTc, preferably said precursor is chosen from the list comprising sialic acid, CMP-sialic acid and lactose, preferably said acceptor is LNnT.
17. Method according to any one of embodiments 6 to 16, the method comprising at least one of the following steps:
   i) Use of a cultivation or incubation medium comprising at least one precursor and/or acceptor;
   ii) Adding to the cultivation or incubation medium in a reactor or incubator at least one precursor and/or acceptor feed wherein the total reactor or incubator volume ranges from 250 mL to 10.000 m³ (cubic meter), preferably in a continuous manner, and preferably so that the final volume of the cultivation or incubation medium is not more than three-fold, preferably not more than two-fold, more preferably less than 2-fold of the volume of the cultivation or incubation medium before the addition of said precursor and/or acceptor feed;
   iii) Adding to the cultivation or incubation medium in a reactor or incubator at least one precursor and/or acceptor feed wherein the total reactor or incubator volume ranges from 250 mL to 10.000 m³ (cubic meter), preferably in a continuous manner, and preferably so that the final volume of the cultivation or incubation medium is not more than three-fold, preferably not more than two-fold, more preferably less than two-fold of the volume of the cultivation or incubation medium before the addition of said precursor and/or acceptor feed and wherein preferably, the pH of said precursor and/or acceptor feed is set between 2.0 and 10.0 and wherein preferably, the temperature of said precursor and/or acceptor feed is kept between 20° C. and 80° C.;
   iv) Adding at least one precursor and/or acceptor feed in a continuous manner to the cultivation or incubation medium over the course of 1 day, 2 days, 3 days, 4 days, 5 days by means of a precursor and/or acceptor feeding solution;
   v) Adding at least one precursor and/or acceptor feed in a continuous manner to the cultivation or incubation medium over the course of 1 day, 2 days, 3 days, 4 days, 5 days by means of a precursor and/or acceptor feeding solution and wherein the concentration of said precursor and/or acceptor feeding solution is 50 g/L, preferably 75 g/L, more preferably 100 g/L, more preferably 125 g/L, more preferably 150 g/L, more preferably 175 g/L, more preferably 200 g/L, more preferably 225 g/L, more preferably 250 g/L, more preferably 275 g/L, more preferably 300 g/L, more preferably 325 g/L, more preferably 350 g/L, more preferably 375 g/L, more preferably, 400 g/L, more preferably 450 g/L, more preferably 500 g/L, even more preferably, 550 g/L, most preferably 600 g/L; and wherein preferably, the pH of said precursor and/or acceptor feeding solution is set between 2.0 and 10.0 and wherein preferably, the temperature of said precursor and/or acceptor feeding solution is kept between 20° C. and 80° C.;

said method resulting in said sialylated oligosaccharide, preferably a 6′sialylated oligosaccharide, more preferably LSTc, with a concentration of at least 50 g/L, preferably at least 75 g/L, more preferably at least 90 g/L, more preferably at least 100 g/L, more preferably at least 125 g/L, more preferably at least 150 g/L, more preferably at least 175 g/L, more preferably at least 200 g/L in the final volume of the cultivation or incubation,

wherein preferably said precursor is chosen from the list comprising sialic acid, CMP-sialic acid and lactose, and wherein preferably said acceptor is LNnT.
18. Method according to any one of embodiments 6 to 16, the method comprising at least one of the following steps:
   i) Use of a cultivation or incubation medium comprising at least 50, more preferably at least 75, more preferably at least 100, more preferably at least 120, more preferably at least 150 grams of precursor per litre of initial reactor or incubator volume wherein the reactor or incubator volume ranges from 250 mL to 10.000 m³ (cubic meter);
   ii) Use of a cultivation or incubation medium comprising at least 50, more preferably at least 75, more preferably at least 100, more preferably at least 120, more preferably at least 150 grams of acceptor per litre of initial reactor or incubator volume wherein the reactor or incubator volume ranges from 250 mL to 10.000 m³ (cubic meter);
   iii) Adding to the cultivation or incubation medium in a reactor or incubator a precursor feed comprising at least 50, more preferably at least 75, more preferably at least 100, more preferably at least 120, more preferably at least 150 grams of precursor per litre of initial reactor or incubator volume wherein the reactor or incubator volume ranges from 250 mL to 10.000 m³ (cubic meter), preferably in a continuous manner, and preferably so that the final volume of the cultivation or incubation medium is not more than three-fold, preferably not more than two-fold, more preferably less than 2-fold of the volume of the cultivation or incubation medium before the addition of said precursor feed;
   iv) Adding to the cultivation or incubation medium in a reactor or incubator an acceptor feed comprising at least 50, more preferably at least 75, more preferably at least 100, more preferably at least 120, more preferably at least 150 grams of acceptor per litre of initial reactor or incubator volume wherein the reactor or incubator volume ranges from 250 mL to 10.000 m³ (cubic meter), preferably in a continuous manner, and preferably so that the final volume of the cultivation or incubation medium is not more than three-fold, preferably not more than two-fold, more preferably less than 2-fold of the volume of the cultivation or incubation medium before the addition of said acceptor feed;
   v) Adding to the cultivation or incubation medium a precursor feed comprising at least 50, more preferably at least 75, more preferably at least 100, more preferably at least 120, more preferably at least 150 grams of precursor per litre of initial reactor or incubator volume wherein the total reactor or incubator volume ranges from 250 mL to 10.000 m³ (cubic meter), preferably in a continuous manner, and preferably so that the final volume of the cultivation or incubation medium is not more than three-fold, preferably not more than two-fold, more preferably less than 2-fold of the volume of the cultivation or incubation medium before the addition of said precursor feed and wherein preferably, the pH of said precursor feed is set between 2.0 and 10.0 and wherein preferably, the temperature of said precursor feed is kept between 20° C. and 80° C.;
   vi) Adding to the cultivation or incubation medium an acceptor feed comprising at least 50, more preferably at least 75, more preferably at least 100, more preferably at least 120, more preferably at least 150 grams of acceptor per litre of initial reactor or incubator volume wherein the total reactor or incubator volume ranges from 250 mL to 10.000 m³ (cubic meter), preferably in a continuous manner, and preferably so that the final volume of the cultivation or incubation medium is not more than three-fold, preferably not more than two-fold, more preferably less than 2-fold of the volume of the cultivation or incubation medium before the addition of said acceptor feed and wherein preferably, the pH of said acceptor feed is set between 2.0 and 10.0 and wherein preferably, the temperature of said acceptor feed is kept between 20° C. and 80° C.;
   vii) Adding a precursor and/or acceptor feed in a continuous manner to the cultivation or incubation medium over the course of 1 day, 2 days, 3 days, 4 days, 5 days by means of a precursor and/or acceptor feeding solution;
   viii) Adding a precursor feed in a continuous manner to the cultivation or incubation medium over the course of 1 day, 2 days, 3 days, 4 days, 5 days by means of a precursor feeding solution and wherein the concentration of said precursor feeding solution is 50 g/L, preferably 75 g/L, more preferably 100 g/L, more preferably 125 g/L, more preferably 150 g/L, more preferably 175 g/L, more preferably 200 g/L, more preferably 225 g/L, more preferably 250 g/L, more preferably 275 g/L, more preferably 300 g/L, more preferably 325 g/L, more preferably 350 g/L, more preferably 375 g/L, more preferably, 400 g/L, more preferably 450 g/L, more preferably 500 g/L, even more preferably, 550 g/L, most preferably 600 g/L; and wherein preferably the pH of said precursor feeding solution is set between 2.0 and 10.0 and wherein preferably, the temperature of said precursor feeding solution is kept between 20° C. and 80° C.;
   ix) Adding an acceptor feed in a continuous manner to the cultivation or incubation medium over the course of 1 day, 2 days, 3 days, 4 days, 5 days by means of an acceptor feeding solution and wherein the concentration of said acceptor feeding solution is 50 g/L, preferably 75 g/L, more preferably 100 g/L, more preferably 125 g/L, more preferably 150 g/L, more preferably 175 g/L, more preferably 200 g/L, more preferably 225 g/L, more preferably 250 g/L, more preferably 275 g/L, more preferably 300 g/L, more preferably 325 g/L, more preferably 350 g/L, more preferably 375 g/L, more preferably, 400 g/L, more preferably 450 g/L, more preferably 500 g/L, even more preferably, 550 g/L, most preferably 600 g/L; and wherein preferably the pH of said acceptor feeding solution is set between 2.0 and 10.0 and wherein preferably, the temperature of said acceptor feeding solution is kept between 20° C. and 80° C.;
   said method resulting in said sialylated oligosaccharide, preferably a 6′sialylated oligosaccharide, more preferably LSTc, with a concentration of at least 50 g/L, preferably at least 75 g/L, more preferably at least 90 g/L, more preferably at least 100 g/L, more preferably at least 125 g/L, more preferably at least 150 g/L, more preferably at least 175 g/L, more preferably at least 200 g/L in the final volume of the cultivation or incubation,
   wherein preferably said precursor is chosen from the list comprising sialic acid, CMP-sialic acid and lactose, and wherein preferably said acceptor is LNnT.
19. A metabolically engineered cell for the production of a sialylated oligosaccharide, preferably a 6′sialylated oligosaccharide, more preferably LSTc (Neu5Ac-α2,6-Gal-β1,4-GlcNAc-β1,3-Gal-β1,4-Glc), wherein said cell has been metabolically engineered to possess, preferably to express, an alpha-2,6-sialyltransferase of any one of embodiments 1 to 5.
20. Cell according to embodiment 19, wherein said cell contains a nucleic acid molecule which comprises a polynucleotide sequence which encodes an alpha-2,6-sialyltransferase of any one of embodiments 1 to 5.
21. Cell according to any one of embodiment 19 or 20, wherein said cell is a bacterium, fungus, yeast, a plant cell, an animal cell, or a protozoan cell,
   preferably said bacterium is an Escherichia coli strain, more preferably an Escherichia coli strain which is a K-12 strain, even more preferably the Escherichia coli K-12 strain is E. coli MG1655,
   preferably said fungus belongs to a genus chosen from the group comprising Rhizopus, Dictyostelium, Penicillium, Mucor or Aspergillus,
   preferably said yeast belongs to a genus chosen from the group comprising Saccharomyces, Zygosaccharomyces, Pichia, Komagataella, Hansenula, Yarrowia, Starmerella, Kluyveromyces or Debaromyces,
   preferably said plant cell is an algal cell or is derived from tobacco, alfalfa, rice, tomato, cotton, rapeseed, soy, maize, or corn plant,
   preferably said animal cell is derived from non-human mammals, birds, fish, invertebrates, reptiles, amphibians or insects or is a genetically engineered cell line derived from human cells excluding embryonic stem cells, more preferably said human and non-human mammalian cell is an epithelial cell, an embryonic kidney cell, a fibroblast cell, a COS cell, a Chinese hamster ovary (CHO) cell, a murine myeloma cell, an NIH-3T3 cell, a non-mammary adult stem cell or derivatives thereof, more preferably said insect cell is derived from Spodoptera frugiperda, Bombyx mori, Mamestra brassicae, Trichoplusia ni or Drosophila melanogaster,
   preferably said protozoan cell is a Leishmania tarentolae cell.
22. Cell according to any one of embodiments 19 to 21, wherein said cell is selected from the group consisting of prokaryotic cells and eukaryotic cells, preferably from the group consisting of yeast cells, bacterial cells, archaebacterial cells, algae cells, and fungal cells.
23. Cell according to any one of embodiments 19 to 22, wherein said cell comprises a nucleic acid molecule comprising a polynucleotide sequence encoding said alpha-2,6-sialyltransferase of any one of embodiments 1 to 5 and operably linked to control sequences recognized by the cell, wherein said sequence is foreign to the cell, said sequence further i) being integrated in the genome of said cell and/or ii) presented to said cell on a vector.
24. Cell according to any one of embodiments 19 to 23, wherein said cell comprises a catabolic pathway for selected mono-, di- or oligosaccharides which is at least partially inactivated, the mono-, di-, or oligosaccharides being involved in and/or required for the synthesis of a sialylated oligosaccharide, preferably a 6′sialylated oligosaccharide, more preferably LSTc.
25. An isolated nucleic acid molecule encoding an alpha-2,6-sialyltransferase of any one of embodiments 1 to 5.
26. A vector comprising the nucleic acid molecule of embodiment 25.
27. Use of an alpha-2,6-sialyltransferase of any one of embodiments 1 to 5 for production of a sialylated oligosaccharide, preferably a 6′sialylated oligosaccharide, more preferably LSTc.
28. Use of a cell according to any one of embodiments 19 to 24 for production of a sialylated oligosaccharide, preferably a 6′sialylated oligosaccharide, more preferably LSTc.
29. Use of a method according to any one of embodiments 6 to 18 for production of a sialylated oligosaccharide, preferably a 6′sialylated oligosaccharide, more preferably LSTc.
30. Use of an isolated nucleic acid molecule according to embodiment 25 for production of a sialylated oligosaccharide, preferably a 6′sialylated oligosaccharide, more preferably LSTc.
31. Use of a vector according to embodiment 26 for production of a sialylated oligosaccharide, preferably a 6′sialylated oligosaccharide, more preferably LSTc.

Moreover, the present invention relates to the following preferred specific embodiments:
1. Method for the production of a 6′sialylated oligosaccharide, preferably LSTc (Neu5Ac-α2,6-Gal-β1,4-GlcNAc-β1,3-Gal-β1,4-Glc), the method comprising: contacting an alpha-2,6-sialyltransferase with a mixture comprising a donor comprising a sialic acid residue, and an acceptor chosen from the list comprising an oligosaccharide or a disaccharide, under conditions where said alpha-2,6-sialyltransferase catalyses the transfer of a sialic acid residue from the donor to the acceptor, thereby producing said 6′sialylated oligosaccharide, characterised in that said alpha-2,6-sialyltransferase is chosen from the list consisting of
   i) an alpha-2,6-sialyltransferase having alpha-2,6-sialyltransferase activity on the terminal galactose (Gal) residue of lacto-N-neotetraose (LNnT, Gal-β1,4-GlcNAc-β1,3-Gal-β1,4-Glc) and comprising an amino acid sequence that is at least 60.0% identical over a stretch of at least 100 amino acid residues, preferably at least 150 amino acid residues, more preferably at least 200 amino acid residues, to the amino acid sequence as represented by SEQ ID NO 1;
   ii) alpha-2,6-sialyltransferase having alpha-2,6-sialyltransferase activity on the terminal galactose (Gal) residue of lacto-N-neotetraose (LNnT, Gal-β1,4-GlcNAc-β1,3-Gal-β1,4-Glc) and comprising an amino acid sequence that is at least 60.0%, at least 65.0%, at least 70.0%, at least 75.0%, at least 80.0%, at least 85.0%, at least 90.0%, at least 95.0%, at least 96.0%, at least 97.0%, at least 98.0%, at least 98.5%, or at least 99.0% identical to the amino acid sequence as represented by SEQ ID NO 1 over a stretch of at least 100, at least 110, at least 120, at least 130, at least 140, at least 150, at least 160, at least 170, at least 180, at least 190, at least 200, at least 210, at least 220, at least 230, at least 240, at least 250, at least 260, at least 270, at least 280, at least 290 or at least 300 amino acid residues;
   iii) alpha-2,6-sialyltransferase having alpha-2,6-sialyltransferase activity on the terminal galactose (Gal) residue of lacto-N-neotetraose (LNnT, Gal-β1,4-GlcNAc-β1,3-Gal-β1,4-Glc) and comprising an amino acid sequence that is at least 60.0%, at least 65.0%, at least 70.0%, at least 75.0%, at least 80.0%, at least 85.0%, at least 90.0%, at least 95.0%, at least 96.0%, at least 97.0%, at least 98.0%, at least 98.5%, or at least 99.0% identical to the full-length amino acid sequence as represented by SEQ ID NO 1; or
   iv) alpha-2,6-sialyltransferase having alpha-2,6-sialyltransferase activity on the terminal galactose (Gal) residue of lacto-N-neotetraose (LNnT, Gal-β1,4-GlcNAc-β1,3-Gal-β1,4-Glc) and wherein said alpha-2,6-sialyltransferase comprises an amino acid sequence as represented by SEQ ID NO 1.
2. Method for the production of a 6′sialylated oligosaccharide, preferably LSTc (Neu5Ac-α2,6-Gal-β1,4-GlcNAc-β1,3-Gal-β1,4-Glc), said method comprising the steps of:
   a) providing
      i. CMP-sialic acid,
      ii. an acceptor, preferably lacto-N-neotetraose (LNnT, Gal-β1,4-GlcNAc-β1,3-Gal-β1,4-Glc), and
      iii. a sialyltransferase,
   b) contacting said sialyltransferase and CMP-sialic acid with said acceptor, preferably LNnT, under conditions where the sialyltransferase catalyses the transfer of a sialic acid residue from said CMP-sialic acid to the acceptor resulting in the production of a 6′sialylated oligosaccharide, preferably LSTc (Neu5Ac-α2,6-Gal-β1,4-GlcNAc-β1,3-Gal-β1,4-Glc),
   c) preferably, separating said produced 6′sialylated oligosaccharide;
   characterised in that sialyltransferase is an alpha-2,6-sialyltransferase chosen from the list consisting of
   i) an alpha-2,6-sialyltransferase having alpha-2,6-sialyltransferase activity on the terminal galactose (Gal) residue of lacto-N-neotetraose (LNnT, Gal-β1,4-GlcNAc-β1,3-Gal-β1,4-Glc) and comprising an amino acid sequence that is at least 60.0% identical over a stretch of at least 100 amino acid residues, preferably at least 150 amino acid residues, more preferably at least 200 amino acid residues, to the amino acid sequence as represented by SEQ ID NO 1;
   ii) alpha-2,6-sialyltransferase having alpha-2,6-sialyltransferase activity on the terminal galactose (Gal) residue of lacto-N-neotetraose (LNnT, Gal-β1,4-GlcNAc-β1,3-Gal-β1,4-Glc) and comprising an amino acid sequence that is at least 60.0%, at least 65.0%, at least 70.0%, at least 75.0%, at least 80.0%, at least 85.0%, at least 90.0%, at least 95.0%, at least 96.0%, at least 97.0%, at least 98.0%, at least 98.5%, or at least 99.0% identical to the amino acid sequence as represented by SEQ ID NO 1 over a stretch of at least 100, at least 110, at least 120, at least 130, at least 140, at least 150, at least 160, at least 170, at least 180, at least 190, at least 200, at least 210, at least 220, at least 230, at least 240, at least 250, at least 260, at least 270, at least 280, at least 290 or at least 300 amino acid residues;
   iii) alpha-2,6-sialyltransferase having alpha-2,6-sialyltransferase activity on the terminal galactose (Gal) residue of lacto-N-neotetraose (LNnT, Gal-β1,4-GlcNAc-β1,3-Gal-β1,4-Glc) and comprising an amino acid sequence that is at least 60.0%, at least 65.0%, at least 70.0%, at least 75.0%, at least 80.0%, at least 85.0%, at least 90.0%, at least 95.0%, at least 96.0%, at least 97.0%, at least 98.0%, at least 98.5%, or at least 99.0% identical to the full-length amino acid sequence as represented by SEQ ID NO 1; or
   iv) alpha-2,6-sialyltransferase having alpha-2,6-sialyltransferase activity on the terminal galactose (Gal) residue of lacto-N-neotetraose (LNnT, Gal-β1,4-GlcNAc-β1,3-Gal-β1,4-Glc) and wherein said alpha-2,6-sialyltransferase comprises an amino acid sequence as represented by SEQ ID NO 1.
3. Method according to any one of embodiments 1 to 2, the method comprising: contacting a cell extract comprising said alpha-2,6-sialyltransferase with a mixture comprising a donor comprising a sialic acid residue, and an acceptor comprising an oligosaccharide or disaccharide, under conditions where said alpha-2,6-sialyltransferase catalyses the transfer of a sialic acid residue from the donor to the acceptor, thereby producing said 6′sialylated oligosaccharide, preferably LSTc.
4. Method according to any one of embodiments 1 to 3, wherein said 6′sialylated oligosaccharide, preferably LSTc, is produced in a cell-free system.
5. Method for the production of a 6′sialylated oligosaccharide, preferably LSTc (Neu5Ac-α2,6-Gal-β1,4-GlcNAc-β1,3-Gal-β1,4-Glc), said method comprising the steps of:
   i. providing a cell, preferably a single cell, expressing, preferably heterologously expressing, more preferably overexpressing, even more preferably heterologously overexpressing, an alpha-2,6-sialyltransferase,
   ii. providing CMP-sialic acid, optionally said CMP-sialic acid is produced by said cell, and
   iii. providing an oligosaccharide or disaccharide, optionally said oligosaccharide or disaccharide is produced by said cell, and
   iv. cultivating and/or incubating said cell under conditions permissive to express said alpha-2,6-sialyltransferase, optionally permissive to produce said CMP-sialic acid and/or said oligosaccharide or disaccharide,
   v. preferably, separating said 6′sialylated oligosaccharide, preferably LSTc, from said cultivation or incubation
   characterised in that said alpha-2,6-sialyltransferase is chosen from the list consisting of
   i) an alpha-2,6-sialyltransferase having alpha-2,6-sialyltransferase activity on the terminal galactose (Gal) residue of lacto-N-neotetraose (LNnT, Gal-β1,4-GlcNAc-β1,3-Gal-β1,4-Glc) and comprising an amino acid sequence that is at least 60.0% identical over a stretch of at least 100 amino acid residues, preferably at least 150 amino acid residues, more preferably at least 200 amino acid residues, to the amino acid sequence as represented by SEQ ID NO 1;
   ii) alpha-2,6-sialyltransferase having alpha-2,6-sialyltransferase activity on the terminal galactose (Gal) residue of lacto-N-neotetraose (LNnT, Gal-β1,4-GlcNAc-β1,3-Gal-β1,4-Glc) and comprising an amino acid sequence that is at least 60.0%, at least 65.0%, at least 70.0%, at least 75.0%, at least 80.0%, at least 85.0%, at least 90.0%, at least 95.0%, at least 96.0%, at least 97.0%, at least 98.0%, at least 98.5%, or at least 99.0% identical to the amino acid sequence as represented by SEQ ID NO 1 over a stretch of at least 100, at least 110, at least 120, at least 130, at least 140, at least 150, at least 160, at least 170, at least 180, at least 190, at least 200, at least 210, at least 220, at least 230, at least 240, at least 250, at least 260, at least 270, at least 280, at least 290 or at least 300 amino acid residues;
   iii) alpha-2,6-sialyltransferase having alpha-2,6-sialyltransferase activity on the terminal galactose (Gal) residue of lacto-N-neotetraose (LNnT, Gal-β1,4-GlcNAc-β1,3-Gal-β1,4-Glc) and comprising an amino acid sequence that is at least 60.0%, at least 65.0%, at least 70.0%, at least 75.0%, at least 80.0%, at least 85.0%, at least 90.0%, at least 95.0%, at least 96.0%, at least 97.0%, at least 98.0%, at least 98.5%, or at least 99.0% identical to the full-length amino acid sequence as represented by SEQ ID NO 1; or
   iv) alpha-2,6-sialyltransferase having alpha-2,6-sialyltransferase activity on the terminal galactose (Gal) residue of lacto-N-neotetraose (LNnT, Gal-β1,4-GlcNAc-β1,3-Gal-β1,4-Glc) and wherein said alpha-2,6-sialyltransferase comprises an amino acid sequence as represented by SEQ ID NO 1.
6. Method according to embodiment 5, wherein said cell is a metabolically engineered cell, preferably wherein said cell is metabolically engineered for the production of said 6′sialylated oligosaccharide, preferably LSTc.
7. Method according to any one of embodiment 5 or 6, wherein the cultivation medium contains at least one carbon source selected from the group consisting of glucose, fructose, sucrose, and glycerol.
8. Method according to any one of embodiments 1 to 7, wherein the cultivation or incubation medium contains at least one compound selected from the group consisting of lactose, galactose, lacto-N-neotetraose (LNnT), UDP-galactose (UDP-Gal), UDP-N-acetylglucosamine (UDP-GlcNAc), sialic acid and CMP-sialic acid.
9. Method according to any one of embodiments 1 to 8, wherein said 6′sialylated oligosaccharide, preferably LSTc, is recovered from the cultivation or incubation medium and/or the cell.
10. Method according to any one of embodiments 1 to 9, the method comprising:
   i) Use of a cultivation or incubation medium comprising at least one precursor and/or acceptor for the production of said 6′sialylated oligosaccharide, preferably LSTc, and/or
   ii) Adding to the cultivation or incubation medium at least one precursor and/or acceptor feed for the production of said 6′sialylated oligosaccharide, preferably LSTc,
   preferably said precursor is chosen from the list comprising sialic acid, CMP-sialic acid and lactose, preferably said acceptor is LNnT.
11. Method according to any one of embodiments 1 to 10, the method comprising at least one of the following steps:
   i) Use of a cultivation or incubation medium comprising at least one precursor and/or acceptor;
   ii) Adding to the cultivation or incubation medium in a reactor or incubator at least one precursor and/or acceptor feed wherein the total reactor or incubator volume ranges from 250 mL to 10.000 m³ (cubic meter), preferably in a continuous manner, and preferably so that the final volume of the cultivation or incubation medium is not more than three-fold, preferably not more than two-fold, more preferably less than 2-fold of the volume of the cultivation or incubation medium before the addition of said precursor and/or acceptor feed;
   iii) Adding to the cultivation or incubation medium in a reactor or incubator at least one precursor and/or acceptor feed wherein the total reactor or incubator volume ranges from 250 mL to 10.000 m³ (cubic meter), preferably in a continuous manner, and preferably so that the final volume of the cultivation or incubation medium is not more than three-fold, preferably not more than two-fold, more preferably less than two-fold of the volume of the cultivation or incubation medium before the addition of said precursor and/or acceptor feed and wherein preferably, the pH of said precursor and/or acceptor feed is set between 2.0 and 10.0 and wherein preferably, the temperature of said precursor and/or acceptor feed is kept between 20° C. and 80° C.;
   iv) Adding at least one precursor and/or acceptor feed in a continuous manner to the cultivation or incubation medium over the course of 1 day, 2 days, 3 days, 4 days, 5 days by means of a precursor and/or acceptor feeding solution;
   v) Adding at least one precursor and/or acceptor feed in a continuous manner to the cultivation or incubation medium over the course of 1 day, 2 days, 3 days, 4 days, 5 days by means of a precursor and/or acceptor feeding solution and wherein the concentration of said precursor and/or acceptor feeding solution is 50 g/L, preferably 75 g/L, more preferably 100 g/L, more preferably 125 g/L, more preferably 150 g/L, more preferably 175 g/L, more preferably 200 g/L, more preferably 225 g/L, more preferably 250 g/L, more preferably 275 g/L, more preferably 300 g/L, more preferably 325 g/L, more preferably 350 g/L, more preferably 375 g/L, more preferably, 400 g/L, more preferably 450 g/L, more preferably 500 g/L, even more preferably, 550 g/L, most preferably 600 g/L; and wherein preferably, the pH of said precursor and/or acceptor feeding solution is set between 2.0 and 10.0 and wherein preferably, the temperature of said precursor and/or acceptor feeding solution is kept between 20° C. and 80° C.;
   said method resulting in said 6′sialylated oligosaccharide, preferably LSTc, with a concentration of at least 50 g/L, preferably at least 75 g/L, more preferably at least 90 g/L, more preferably at least 100 g/L, more preferably at least 125 g/L, more preferably at least 150 g/L, more preferably at least 175 g/L, more preferably at least 200 g/L in the final volume of the cultivation or incubation,
   wherein preferably said precursor is chosen from the list comprising sialic acid, CMP-sialic acid and lactose, and wherein preferably said acceptor is LNnT.
12. Method according to any one of embodiments 1 to 10, the method comprising at least one of the following steps:
   i) Use of a cultivation or incubation medium comprising at least 50, more preferably at least 75, more preferably at least 100, more preferably at least 120, more preferably at least 150 grams of precursor per litre of initial reactor or incubator volume wherein the reactor or incubator volume ranges from 250 mL to 10.000 m³ (cubic meter);
   ii) Use of a cultivation or incubation medium comprising at least 50, more preferably at least 75, more preferably at least 100, more preferably at least 120, more preferably at least 150 grams of acceptor per litre of initial reactor or incubator volume wherein the reactor or incubator volume ranges from 250 mL to 10.000 m³ (cubic meter);
   iii) Adding to the cultivation or incubation medium in a reactor or incubator a precursor feed comprising at least 50, more preferably at least 75, more preferably at least 100, more preferably at least 120, more preferably at least 150 grams of precursor per litre of initial reactor or incubator volume wherein the reactor or incubator volume ranges from 250 mL to 10.000 m³ (cubic meter), preferably in a continuous manner, and preferably so that the final volume of the cultivation or incubation medium is not more than three-fold, preferably not more than two-fold, more preferably less than 2-fold of the volume of the cultivation or incubation medium before the addition of said precursor feed;
   iv) Adding to the cultivation or incubation medium in a reactor or incubator an acceptor feed comprising at least 50, more preferably at least 75, more preferably at least 100, more preferably at least 120, more preferably at least 150 grams of acceptor per litre of initial reactor or incubator volume wherein the reactor or incubator volume ranges from 250 mL to 10.000 m³ (cubic meter), preferably in a continuous manner, and preferably so that the final volume of the cultivation or incubation medium is not more than three-fold, preferably not more than two-fold, more preferably less than 2-fold of the volume of the cultivation or incubation medium before the addition of said acceptor feed;
   v) Adding to the cultivation or incubation medium a precursor feed comprising at least 50, more preferably at least 75, more preferably at least 100, more preferably at least 120, more preferably at least 150 grams of precursor per litre of initial reactor or incubator volume wherein the total reactor or incubator volume ranges from 250 mL to 10.000 m³ (cubic meter), preferably in a continuous manner, and preferably so that the final volume of the cultivation or incubation medium is not more than three-fold, preferably not more than two-fold, more preferably less than 2-fold of the volume of the cultivation or incubation medium before the addition of said precursor feed and wherein preferably, the pH of said precursor feed is set between 2.0 and 10.0 and wherein preferably, the temperature of said precursor feed is kept between 20° C. and 80° C.;
   vi) Adding to the cultivation or incubation medium an acceptor feed comprising at least 50, more preferably at least 75, more preferably at least 100, more preferably at least 120, more preferably at least 150 grams of acceptor per litre of initial reactor or incubator volume wherein the total reactor or incubator volume ranges from 250 mL to 10.000 m³ (cubic meter), preferably in a continuous manner, and preferably so that the final volume of the cultivation or incubation medium is not more than three-fold, preferably not more than two-fold, more preferably less than 2-fold of the volume of the cultivation or incubation medium before the addition of said acceptor feed and wherein preferably, the pH of said acceptor feed is set between 2.0 and 10.0 and wherein preferably, the temperature of said acceptor feed is kept between 20° C. and 80° C.;
   vii) Adding a precursor and/or acceptor feed in a continuous manner to the cultivation or incubation medium over the course of 1 day, 2 days, 3 days, 4 days, 5 days by means of a precursor and/or acceptor feeding solution;
   viii) Adding a precursor feed in a continuous manner to the cultivation or incubation medium over the course of 1 day, 2 days, 3 days, 4 days, 5 days by means of a precursor feeding solution and wherein the concentration of said precursor feeding solution is 50 g/L, preferably 75 g/L, more preferably 100 g/L, more preferably 125 g/L, more preferably 150 g/L, more preferably 175 g/L, more preferably 200 g/L, more preferably 225 g/L, more preferably 250 g/L, more preferably 275 g/L, more preferably 300 g/L, more preferably 325 g/L, more preferably 350 g/L, more preferably 375 g/L, more preferably, 400 g/L, more preferably 450 g/L, more preferably 500 g/L, even more preferably, 550 g/L, most preferably 600 g/L; and wherein preferably the pH of said precursor feeding solution is set between 2.0 and 10.0 and wherein preferably, the temperature of said precursor feeding solution is kept between 20° C. and 80° C.;
   ix) Adding an acceptor feed in a continuous manner to the cultivation or incubation medium over the course of 1 day, 2 days, 3 days, 4 days, 5 days by means of an acceptor feeding solution and wherein the concentration of said acceptor feeding solution is 50 g/L, preferably 75 g/L, more preferably 100 g/L, more preferably 125 g/L, more preferably 150 g/L, more preferably 175 g/L, more preferably 200 g/L, more preferably 225 g/L, more preferably 250 g/L, more preferably 275 g/L, more preferably 300 g/L, more preferably 325 g/L, more preferably 350 g/L, more preferably 375 g/L, more preferably, 400 g/L, more preferably 450 g/L, more preferably 500 g/L, even more preferably, 550 g/L, most preferably 600 g/L; and wherein preferably the pH of said acceptor feeding solution is set between 2.0 and 10.0 and wherein preferably, the temperature of said acceptor feeding solution is kept between 20° C. and 80° C.;
   said method resulting in said 6′sialylated oligosaccharide, preferably LSTc, with a concentration of at least 50 g/L, preferably at least 75 g/L, more preferably at least 90 g/L, more preferably at least 100 g/L, more preferably at least 125 g/L, more preferably at least 150 g/L, more preferably at least 175 g/L, more preferably at least 200 g/L in the final volume of the cultivation or incubation, wherein preferably said precursor is chosen from the list comprising sialic acid, CMP-sialic acid and lactose, and wherein preferably said acceptor is LNnT.
13. Method according to any one of embodiment 1 to 12, wherein said 6′sialylated oligosaccharide is LSTc (Neu5Ac-α2,6-Gal-β1,4-GlcNAc-β1,3-Gal-β1,4-Glc) and said acceptor is lacto-N-neotetraose (LNnT, Gal-β1,4-GlcNAc-β1,3-Gal-β1,4-Glc).
14. A metabolically engineered cell for the production of a 6′sialylated oligosaccharide, preferably LSTc (Neu5Ac-α2,6-Gal-β1,4-GlcNAc-β1,3-Gal-β1,4-Glc), wherein said cell has been metabolically engineered to possess, preferably to express, an alpha-2,6-sialyltransferase chosen from the list consisting of
   i) an alpha-2,6-sialyltransferase having alpha-2,6-sialyltransferase activity on the terminal galactose (Gal) residue of lacto-N-neotetraose (LNnT, Gal-β1,4-GlcNAc-β1,3-Gal-β1,4-Glc) and comprising an amino acid sequence that is at least 60.0% identical over a stretch of at least 100 amino acid residues, preferably at least 150 amino acid residues, more preferably at least 200 amino acid residues, to the amino acid sequence as represented by SEQ ID NO 1;
   ii) alpha-2,6-sialyltransferase having alpha-2,6-sialyltransferase activity on the terminal galactose (Gal) residue of lacto-N-neotetraose (LNnT, Gal-β1,4-GlcNAc-β1,3-Gal-β1,4-Glc) and comprising an amino acid sequence that is at least 60.0%, at least 65.0%, at least 70.0%, at least 75.0%, at least 80.0%, at least 85.0%, at least 90.0%, at least 95.0%, at least 96.0%, at least 97.0%, at least 98.0%, at least 98.5%, or at least 99.0% identical to the amino acid sequence as represented by SEQ ID NO 1 over a stretch of at least 100, at least 110, at least 120, at least 130, at least 140, at least 150, at least 160, at least 170, at least 180, at least 190, at least 200, at least 210, at least 220, at least 230, at least 240, at least 250, at least 260, at least 270, at least 280, at least 290 or at least 300 amino acid residues;
   iii) alpha-2,6-sialyltransferase having alpha-2,6-sialyltransferase activity on the terminal galactose (Gal) residue of lacto-N-neotetraose (LNnT, Gal-β1,4-GlcNAc-β1,3-Gal-β1,4-Glc) and comprising an amino acid sequence that is at least 60.0%, at least 65.0%, at least 70.0%, at least 75.0%, at least 80.0%, at least 85.0%, at least 90.0%, at least 95.0%, at least 96.0%, at least 97.0%, at least 98.0%, at least 98.5%, or at least 99.0% identical to the full-length amino acid sequence as represented by SEQ ID NO 1; or
   iv) alpha-2,6-sialyltransferase having alpha-2,6-sialyltransferase activity on the terminal galactose (Gal) residue of lacto-N-neotetraose (LNnT, Gal-β1,4-GlcNAc-β1,3-Gal-β1,4-Glc) and wherein said alpha-2,6-sialyltransferase comprises an amino acid sequence as represented by SEQ ID NO 1.
15. Cell according to embodiment 14, wherein said cell contains a nucleic acid molecule which comprises a polynucleotide sequence which encodes said alpha-2,6-sialyltransferase.
16. Cell according to any one of embodiment 14 or 15, wherein said cell is a bacterium, fungus, yeast, a plant cell, an animal cell, or a protozoan cell,
   preferably said bacterium is an Escherichia coli strain, more preferably an Escherichia coli strain which is a K-12 strain, even more preferably the Escherichia coli K-12 strain is E. coli MG1655,
   preferably said fungus belongs to a genus chosen from the group comprising Rhizopus, Dictyostelium, Penicillium, Mucor or Aspergillus,
   preferably said yeast belongs to a genus chosen from the group comprising Saccharomyces, Zygosaccharomyces, Pichia, Komagataella, Hansenula, Yarrowia, Starmerella, Kluyveromyces or Debaromyces,
   preferably said plant cell is an algal cell or is derived from tobacco, alfalfa, rice, tomato, cotton, rapeseed, soy, maize, or corn plant,
   preferably said animal cell is derived from non-human mammals, birds, fish, invertebrates, reptiles, amphibians or insects or is a genetically engineered cell line derived from human cells excluding embryonic stem cells, more preferably said human and non-human mammalian cell is an epithelial cell, an embryonic kidney cell, a fibroblast cell, a COS cell, a Chinese hamster ovary (CHO) cell, a murine myeloma cell, an NIH-3T3 cell, a non-mammary adult stem cell or derivatives thereof, more preferably said insect cell is derived from Spodoptera frugiperda, Bombyx mori, Mamestra brassicae, Trichoplusia ni or Drosophila melanogaster,
   preferably said protozoan cell is a Leishmania tarentolae cell.
17. Cell according to any one of embodiments 14 to 16, wherein said cell is selected from the group consisting of prokaryotic cells and eukaryotic cells, preferably from the group consisting of yeast cells, bacterial cells, archaebacterial cells, algae cells, and fungal cells.
18. Cell according to any one of embodiments 14 to 17, wherein said cell is metabolically engineered to comprise any one or more gene encoding a polypeptide involved in the pathway for production of a sialylated di- and/or oligosaccharide.
19. Cell according to any one of embodiments 14 to 18, wherein said cell comprises a nucleic acid molecule comprising a polynucleotide sequence encoding said alpha-2,6-sialyltransferase and operably linked to control sequences recognized by the cell, wherein said sequence is foreign to the cell, said sequence further i) being integrated in the genome of said cell and/or ii) presented to said cell on a vector.
20. Cell according to any one of embodiments 14 to 19, wherein said cell comprises a catabolic pathway for selected mono-, di- or oligosaccharides which is at least partially inactivated, the mono-, di-, or oligosaccharides being involved in and/or required for the synthesis of a 6′sialylated oligosaccharide, preferably LSTc.
21. A vector comprising a nucleic acid molecule which encodes an alpha-2,6-sialyltransferase chosen from the list consisting of
   i) an alpha-2,6-sialyltransferase having alpha-2,6-sialyltransferase activity on the terminal galactose (Gal) residue of lacto-N-neotetraose (LNnT, Gal-β1,4-GlcNAc-β1,3-Gal-β1,4-Glc) and comprising an amino acid sequence that is at least 60.0% identical over a stretch of at least 100 amino acid residues, preferably at least 150 amino acid residues, more preferably at least 200 amino acid residues, to the amino acid sequence as represented by SEQ ID NO 1;
   ii) alpha-2,6-sialyltransferase having alpha-2,6-sialyltransferase activity on the terminal galactose (Gal) residue of lacto-N-neotetraose (LNnT, Gal-β1,4-GlcNAc-β1,3-Gal-β1,4-Glc) and comprising an amino acid sequence that is at least 60.0%, at least 65.0%, at least 70.0%, at least 75.0%, at least 80.0%, at least 85.0%, at least 90.0%, at least 95.0%, at least 96.0%, at least 97.0%, at least 98.0%, at least 98.5%, or at least 99.0% identical to the amino acid sequence as represented by SEQ ID NO 1 over a stretch of at least 100, at least 110, at least 120, at least 130, at least 140, at least 150, at least 160, at least 170, at least 180, at least 190, at least 200, at least 210, at least 220, at least 230, at least 240, at least 250, at least 260, at least 270, at least 280, at least 290 or at least 300 amino acid residues;
   iii) alpha-2,6-sialyltransferase having alpha-2,6-sialyltransferase activity on the terminal galactose (Gal) residue of lacto-N-neotetraose (LNnT, Gal-β1,4-GlcNAc-β1,3-Gal-β1,4-Glc) and comprising an amino acid sequence that is at least 60.0%, at least 65.0%, at least 70.0%, at least 75.0%, at least 80.0%, at least 85.0%, at least 90.0%, at least 95.0%, at least 96.0%, at least 97.0%, at least 98.0%, at least 98.5%, or at least 99.0% identical to the full-length amino acid sequence as represented by SEQ ID NO 1; or
   iv) alpha-2,6-sialyltransferase having alpha-2,6-sialyltransferase activity on the terminal galactose (Gal) residue of lacto-N-neotetraose (LNnT, Gal-β1,4-GlcNAc-β1,3-Gal-β1,4-Glc) and wherein said alpha-2,6-sialyltransferase comprises an amino acid sequence as represented by SEQ ID NO 1.
22. Use of an alpha-2,6-sialyltransferase for production of a 6′sialylated oligosaccharide, preferably LSTc, wherein said alpha-2,6-sialyltransferase is chosen from the list consisting of
   i) an alpha-2,6-sialyltransferase having alpha-2,6-sialyltransferase activity on the terminal galactose (Gal) residue of lacto-N-neotetraose (LNnT, Gal-β1,4-GlcNAc-β1,3-Gal-β1,4-Glc) and comprising an amino acid sequence that is at least 60.0% identical over a stretch of at least 100 amino acid residues, preferably at least 150 amino acid residues, more preferably at least 200 amino acid residues, to the amino acid sequence as represented by SEQ ID NO 1;
   ii) alpha-2,6-sialyltransferase having alpha-2,6-sialyltransferase activity on the terminal galactose (Gal) residue of lacto-N-neotetraose (LNnT, Gal-β1,4-GlcNAc-β1,3-Gal-β1,4-Glc) and comprising an amino acid sequence that is at least 60.0%, at least 65.0%, at least 70.0%, at least 75.0%, at least 80.0%, at least 85.0%, at least 90.0%, at least 95.0%, at least 96.0%, at least 97.0%, at least 98.0%, at least 98.5%, or at least 99.0% identical to the amino acid sequence as represented by SEQ ID NO 1 over a stretch of at least 100, at least 110, at least 120, at least 130, at least 140, at least 150, at least 160, at least 170, at least 180, at least 190, at least 200, at least 210, at least 220, at least 230, at least 240, at least 250, at least 260, at least 270, at least 280, at least 290 or at least 300 amino acid residues;
   iii) alpha-2,6-sialyltransferase having alpha-2,6-sialyltransferase activity on the terminal galactose (Gal) residue of lacto-N-neotetraose (LNnT, Gal-β1,4-GlcNAc-β1,3-Gal-β1,4-Glc) and comprising an amino acid sequence that is at least 60.0%, at least 65.0%, at least 70.0%, at least 75.0%, at least 80.0%, at least 85.0%, at least 90.0%, at least 95.0%, at least 96.0%, at least 97.0%, at least 98.0%, at least 98.5%, or at least 99.0% identical to the full-length amino acid sequence as represented by SEQ ID NO 1; or
   iv) alpha-2,6-sialyltransferase having alpha-2,6-sialyltransferase activity on the terminal galactose (Gal) residue of lacto-N-neotetraose (LNnT, Gal-β1,4-GlcNAc-β1,3-Gal-β1,4-Glc) and wherein said alpha-2,6-sialyltransferase comprises an amino acid sequence as represented by SEQ ID NO 1 for production of a a 6′sialylated oligosaccharide, preferably LSTc.
23. Use of a cell according to any one of embodiments 14 to 20 for production of a 6′sialylated oligosaccharide, preferably LSTc.
24. Use of a method according to any one of embodiments 1 to 13 for production of a 6′sialylated oligosaccharide, preferably LSTc.
25. Use of an isolated nucleic acid molecule which encodes an alpha-2,6-sialyltransferase for production of a 6′sialylated oligosaccharide, preferably LSTc, wherein said alpha-2,6-sialyltransferase is chosen from the list consisting of
   i) an alpha-2,6-sialyltransferase having alpha-2,6-sialyltransferase activity on the terminal galactose (Gal) residue of lacto-N-neotetraose (LNnT, Gal-β1,4-GlcNAc-β1,3-Gal-β1,4-Glc) and comprising an amino acid sequence that is at least 60.0% identical over a stretch of at least 100 amino acid residues, preferably at least 150 amino acid residues, more preferably at least 200 amino acid residues, to the amino acid sequence as represented by SEQ ID NO 1;
   ii) alpha-2,6-sialyltransferase having alpha-2,6-sialyltransferase activity on the terminal galactose (Gal) residue of lacto-N-neotetraose (LNnT, Gal-β1,4-GlcNAc-β1,3-Gal-β1,4-Glc) and comprising an amino acid sequence that is at least 60.0%, at least 65.0%, at least 70.0%, at least 75.0%, at least 80.0%, at least 85.0%, at least 90.0%, at least 95.0%, at least 96.0%, at least 97.0%, at least 98.0%, at least 98.5%, or at least 99.0% identical to the amino acid sequence as represented by SEQ ID NO 1 over a stretch of at least 100, at least 110, at least 120, at least 130, at least 140, at least 150, at least 160, at least 170, at least 180, at least 190, at least 200, at least 210, at least 220, at least 230, at least 240, at least 250, at least 260, at least 270, at least 280, at least 290 or at least 300 amino acid residues;
   iii) alpha-2,6-sialyltransferase having alpha-2,6-sialyltransferase activity on the terminal galactose (Gal) residue of lacto-N-neotetraose (LNnT, Gal-β1,4-GlcNAc-β1,3-Gal-β1,4-Glc) and comprising an amino acid sequence that is at least 60.0%, at least 65.0%, at least 70.0%, at least 75.0%, at least 80.0%, at least 85.0%, at least 90.0%, at least 95.0%, at least 96.0%, at least 97.0%, at least 98.0%, at least 98.5%, or at least 99.0% identical to the full-length amino acid sequence as represented by SEQ ID NO 1; or
   iv) alpha-2,6-sialyltransferase having alpha-2,6-sialyltransferase activity on the terminal galactose (Gal) residue of lacto-N-neotetraose (LNnT, Gal-β1,4-GlcNAc-β1,3-Gal-β1,4-Glc) and wherein said alpha-2,6-sialyltransferase comprises an amino acid sequence as represented by SEQ ID NO 1.
26. Use of a vector according to embodiment 21 for production of a 6′sialylated oligosaccharide, preferably LSTc.

The invention will be described in more detail in the examples. The following examples will serve as further illustration and clarification of the present invention and are not intended to be limiting.

### EXAMPLES

### Example 1. Calculation of percentage identity between nucleotide or polypeptide sequences

### Heterologous and Homologous Expression

Genes that needed to be expressed, be it from a plasmid or from the genome were synthetically synthetized with one of the following companies: IDT or Twist Bioscience. Proteins described in present disclosure are summarized in Tables 1 and 2. Unless stated otherwise, the UniProt IDs of the proteins described correspond to their sequence version 01 as present in the UniProt Database version release 2021_03 of 9 Jun. 2021. Expression could be further facilitated by optimizing the codon usage to the codon usage of the expression host. Genes were optimized using the tools of the supplier.

TABLE 1
Overview of proteins with corresponding SEQ ID NOs or UniProt IDs described in present invention

| | | | | Country of origin of digital |
|---|---|---|---|---|
| SEQ ID NO | Name | Organism | Origin | sequence information |
| 01 | HparaST | Haemophilus paracuniculus | Synthetic | USA |
| 02 | neuA | Pasteurella multocida | Synthetic | USA |
| P43577 | GNA1 | Saccharomyces cerevisiae | Synthetic | USA |
| A7LVG6 | AGE | Bacteroides ovatus | Synthetic | USA |
| E0NCD4 | neuB | Neisseria meningitidis | Synthetic | United Kingdom |
| Q93MP9 | neuB | Campylobacter jejuni | Synthetic | USA |
| Q93MP8 | neuC | Campylobacter jejuni | Synthetic | USA |
| P31120 * | glmM | E. coli K-12 MG1655 | Synthetic | USA |
| P0ACC7 | glmU | E. coli K-12 MG1655 | Synthetic | USA |
| Q91WG8 | GNE | Mus musculus (strain C57BL/6J) | Synthetic | USA |
| K9NPH9 | NANS | Pseudomonas sp. UW4 | Synthetic | Unknown |
| KPA15328.1 | NANP | Candidatus Magnetomorum sp. HK-1 | Synthetic | Germany |
| Q8A712 | NANP | Bacteroides thetaiotaomicron | Synthetic | Unknown |
| P17169 ** | glmS | E. coli K-12 MG1655 | Synthetic | USA |
| P27550 | acs | E. coli K-12 MG1655 | Synthetic | USA |
| O66375 | PdST6 | Photobacterium damselae | Synthetic | Japan |
| P02920 | LacY | E. coli K-12 MG1655 | Synthetic | USA |
| E0IXR1 | cscB | E. coli W | Synthetic | USA |
| Q03417 | Frk | Zymomonas mobilis | Synthetic | United Kingdom |
| A0ZZH6 | BaSP | Bifidobacterium adolescentis | Synthetic | Germany |
| Q9JXQ6 | IgtA | Neisseria meningitidis MC58 | Synthetic | United Kingdom |
| Q51116 | IgtB | Neisseria meningitidis MC58 | Synthetic | United Kingdom |
| P09147 | galE | E. coli K-12 MG1655 | Synthetic | USA |
| P0A840 | surE | E. coli K-12 MG1655 | Synthetic | USA |
| P07921 | LAC12 | Kluyveromyces lactis | Synthetic | USA |
| P0CI73 | glmS | Bacillus subtilis | Synthetic | USA |
| Q8NND3 *** | glmS | Corynebacterium glutamicum | Synthetic | USA |
| Q9SEE5 | KIN | Arabidopsis thaliana | Synthetic | USA |
| Q9C5I1 | USP | Arabidopsis thaliana | Synthetic | USA |
| Q9Y223 | GNE | Homo sapiens | Synthetic | Unknown |
| Q9NR45 | NANS | Homo sapiens | Synthetic | Unknown |
| Q8NFW8 | CMAS | Homo sapiens | Synthetic | Unknown |
| Q61420 | CST | Mus musculus | Synthetic | USA |
| Q06210 | GFPT1 | Homo sapiens | Synthetic | Unknown |
| Q96EK6 | GNA1 | Homo sapiens | Synthetic | Unknown |
| O95394 | PGM3 | Homo sapiens | Synthetic | Unknown |
| Q16222 | UAP1 | Homo sapiens | Synthetic | Unknown |
| Q99KK2 | neuA | Mus musculus | Synthetic | USA |

* Sequence version 03 (23 Jan. 2007) as present in the UniProt Database 2021_03 of 9 Jun. 2021
** Sequence version 04 (23 Jan. 2007) as present in the UniProt Database 2021_03 of 9 Jun. 2021
*** Sequence version 02 (23 Jan. 2007) as present in the UniProt Database 2021_03 of 9 Jun. 2021

### Calculation of Percentage Identity Between Nucleotide or Polypeptide Sequences

Methods for the alignment of sequences for comparison are well known in the art, such methods include GAP, BESTFIT, BLAST, FASTA and TFASTA. GAP uses the algorithm of Needleman and Wunsch (J. Mol. Biol. (1970) 48: 443-453) to find the global (i.e. spanning the full-length sequences) alignment of two sequences that maximizes the number of matches and minimizes the number of gaps. The BLAST algorithm (Altschul et al., J. Mol. Biol. (1990) 215: 403-10) calculates the global percentage sequence identity (i.e. over the full-length sequence) and performs a statistical analysis of the similarity between the two sequences. The software for performing BLAST analysis is publicly available through the National Centre for Biotechnology Information (NCBI). Homologs may readily be identified using, for example, the ClustalW multiple sequence alignment algorithm (version 1.83), with the default pairwise alignment parameters, and a scoring method in percentage. Global percentages of similarity and identity (i.e. spanning the full-length sequences) may also be determined using one of the methods available in the MatGAT software package (Campanella et al., BMC Bioinformatics (2003) 4:29). Minor manual editing may be performed to optimize alignment between conserved motifs, as would be apparent to a person skilled in the art. Furthermore, instead of using full-length sequences for the identification of homologs, specific domains may also be used, to determine the so-called local sequence identity. The sequence identity values may be determined over the entire nucleic acid or amino acid sequence (=local sequence identity search over the full-length sequence resulting in a global sequence identity score) or over selected domains or conserved motif(s) (=local sequence identity search over a partial sequence resulting in a local sequence identity score), using the programs mentioned above using the default parameters. For local alignments, the Smith-Waterman algorithm is particularly useful (Smith T F, Waterman M S (1981) J. Mol. Biol 147(1); 195-7).

### Optical Density

Cell density of the cultures was frequently monitored by measuring optical density at 600 nm (Implen Nanophotometer NP80, Westburg, Belgium or with a Spark 10M microplate reader, Tecan, Switzerland). The maximum growth speed (mumax) was calculated based on the observed optical densities at 600 nm using the R package grofit.

### Analytical Analysis

Standards such as but not limited to sucrose, lactose, LacNAc, lacto-N-biose (LNB), fucosylated LacNAc (2′FLacNAc, 3-FLacNAc), sialylated LacNAc, (3′SLacNAc, 6′SLacNAc), fucosylated LNB (2′FLNB, 4′FLNB), lacto-N-triose II (LN3), lacto-N-tetraose (LNT), lacto-N-neo-tetraose (LNnT), LNFP-I, LNFP-II, LNFP-III, LNFP-V, LSTa, LSTc and LSTd were purchased from Carbosynth (UK), Elicityl (France) and IsoSep (Sweden). Other compounds were analyzed with in-house made standards.

Neutral oligosaccharides were analyzed on a Waters Acquity H-class UPLC with Evaporative Light Scattering Detector (ELSD) or a Refractive Index (RI) detection. A volume of 0.7 μL sample was injected on a Waters Acquity UPLC BEH Amide column (2.1×100 mm; 130 Å; 1.7 μm) column with an Acquity UPLC BEH Amide VanGuard column, 130 Å, 2.1×5 mm. The column temperature was 50° C. The mobile phase consisted of a ¼ water and % acetonitrile solution to which 0.2% triethylamine was added. The method was isocratic with a flow of 0.130 mL/min. The ELS detector had a drift tube temperature of 50° C. and the N2 gas pressure was 50 psi, the gain 200 and the data rate 10 pps. The temperature of the RI detector was set at 35° C.

Sialylated oligosaccharides were analyzed on a Waters Acquity H-class UPLC with Refractive Index (RI) detection. A volume of 0.5 μL sample was injected on a Waters Acquity UPLC BEH Amide column (2.1×100 mm; 130 Å; 1.7 μm). The column temperature was 50° C. The mobile phase consisted of a mixture of 70% acetonitrile, 26% ammonium acetate buffer (150 mM) and 4% methanol to which 0.05% pyrrolidine was added. The method was isocratic with a flow of 0.150 mL/min. The temperature of the RI detector was set at 35° C.

Both neutral and sialylated sugars were analyzed on a Waters Acquity H-class UPLC with Refractive Index (RI) detection. A volume of 0.5 μL sample was injected on a Waters Acquity UPLC BEH Amide column (2.1 ×100 mm; 130 Å; 1.7 μm). The column temperature was 50° C. The mobile phase consisted of a mixture of 72% acetonitrile and 28% ammonium acetate buffer (100 mM) to which 0.1% triethylamine was added.

The method was isocratic with a flow of 0.260 mL/min. The temperature of the RI detector was set at 35° C.

For analysis on a mass spectrometer, a Waters Xevo TQ-MS with Electron Spray Ionisation (ESI) was used with a desolvation temperature of 450° C., a nitrogen desolvation gas flow of 650 L/h and a cone voltage of 20 V. The MS was operated in selected ion monitoring (SIM) in negative mode for all oligosaccharides. Separation was performed on a Waters Acquity UPLC with a Thermo Hypercarb column (2.1×100 mm; 3 μm) on 35° C. A gradient was used wherein eluent A was ultrapure water with 0.1% formic acid and wherein eluent B was acetonitrile with 0.1% formic acid. The oligosaccharides were separated in 55 min using the following gradient: an initial increase from 2 to 12% of eluent B over 21 min, a second increase from 12 to 40% of eluent B over 11 min and a third increase from 40 to 100% of eluent B over 5 min. As a washing step 100% of eluent B was used for 5 min. For column equilibration, the initial condition of 2% of eluent B was restored in 1 min and maintained for 12 min.

Both neutral and sialylated sugars at low concentrations (below 50 mg/L) were analyzed on a Dionex HPAEC system with pulsed amperometric detection (PAD). A volume of 5 μL of sample was injected on a Dionex CarboPac PA200 column 4×250 mm with a Dionex CarboPac PA200 guard column 4×50 mm. The column temperature was set to 30° C. A gradient was used wherein eluent A was deionized water, wherein eluent B was 200 mM Sodium hydroxide and wherein eluent C was 500 mM Sodium acetate. The oligosaccharides were separated in 60 min while maintaining a constant ratio of 25% of eluent B using the following gradient: an initial isocratic step maintained for 10 min of 75% of eluent A, an initial increase from 0 to 4% of eluent C over 8 min, a second isocratic step maintained for 6 min of 71% of eluent A and 4% of eluent C, a second increase from 4 to 12% of eluent C over 2.6 min, a third isocratic step maintained for 3.4 min of 63% of eluent A and 12% of eluent C and a third increase from 12 to 48% of eluent C over 5 min. As a washing step 48% of eluent C was used for 3 min. For column equilibration, the initial condition of 75% of eluent A and 0% of eluent C was restored in 1 min and maintained for 11 min. The applied flow was 0.5 mL/min.

### Example 2. Materials and Methods Escherichia coli

### Media

The Luria Broth (LB) medium consisted of 1% tryptone peptone (Difco, Erembodegem, Belgium), 0.5% yeast extract (Difco) and 0.5% sodium chloride (VWR. Leuven, Belgium). The minimal medium used in the cultivation experiments in 96-well plates or in shake flasks contained 2.00 g/L NH₄Cl, 5.00 g/L (NH₄)₂SO₄, 2.993 g/L KH₂PO₄, 7.315 g/L K₂HPO₄, 8.372 g/L MOPS, 0.5 g/L NaCl, 0.5 g/L MgSO₄·7H₂O, 30 g/L sucrose or 30 g/L glycerol, 1 ml/L vitamin solution, 100 μl/L molybdate solution, and 1 mL/L selenium solution. As specified in the respective examples, 0.30 g/L sialic acid and/or 20 g/L lactose were additionally added to the medium. The minimal medium was set to a pH of 7.0 with 1M KOH. Vitamin solution consisted of 3.6 g/L FeCl₂·4H₂O, 5.0 g/L CaCl₂·2H₂O, 1.3 g/L MnCl₂·2H₂O, 0.38 g/L CuCl₂·2H₂O, 0.5 g/L CoCl₂·6H₂O, 0.94 g/L ZnCl₂, 0.0311 g/L H₃BO₄, 0.4 g/L Na₂EDTA·2H₂O and 1.01 g/L thiamine·HCl. The molybdate solution contained 0.967 g/L NaMoO₄·2H₂O. The selenium solution contained 42 g/L Seo₂.

The minimal medium for fermentations contained 6.75 g/L NH₄Cl, 1.25 g/L (NH₄)₂SO₄, 2.93 g/L KH₂PO₄ and 7.31 g/L KH₂PO₄, 0.5 g/L NaCl, 0.5 g/L MgSO₄·7H₂O, 30 g/L sucrose or 30 g/L glycerol, 1 mL/L vitamin solution, 100 μL/L molybdate solution, and 1 mL/L selenium solution with the same composition as described above. As specified in the respective examples, 0.30 g/L sialic acid and/or 20 g/L lactose were additionally added to the medium.

Complex medium was sterilized by autoclaving (121° C., 21 min) and minimal medium by filtration (0.22 μm Sartorius). When necessary, the medium was made selective by adding an antibiotic: e.g. chloramphenicol (20 mg/L), carbenicillin (100 mg/L), spectinomycin (40 mg/L) and/or kanamycin (50 mg/L).

### Plasmids

pKD46 (Red helper plasmid, Ampicillin resistance), pKD3 (contains an FRT-flanked chloramphenicol resistance (cat) gene), pKD4 (contains an FRT-flanked kanamycin resistance (kan) gene), and pCP20 (expresses FLP recombinase activity) plasmids were obtained from Prof. R. Cunin (Vrije Universiteit Brussel, Belgium in 2007). Plasmids were maintained in the host E. coli DH5alpha (F⁻, phi80dlacZΔM15, Δ(lacZYA-argF) U169, deoR, recA1, endA1, hsdR17(rk⁻, mk⁺), phoA, supE44, lambda-, thi-1, gyrA96, re/A1) bought from Invitrogen.

### Strains and Mutations

Escherichia coli K12 MG1655 [λ⁻, F⁻, rph-1] was obtained from the Coli Genetic Stock Center (US), CGSC Strain #: 7740, in March 2007. Gene disruptions, gene introductions and gene replacements were performed using the technique published by Datsenko and Wanner (PNAS 97 (2000), 6640-6645). This technique is based on antibiotic selection after homologous recombination performed by lambda Red recombinase. Subsequent catalysis of a flippase recombinase ensures removal of the antibiotic selection cassette in the final production strain. Transformants carrying a Red helper plasmid pKD46 were grown in 10 mL LB media with ampicillin, (100 mg/L) and L-arabinose (10 mM) at 30° C. to an OD_{600 nm} of 0.6. The cells were made electrocompetent by washing them with 50 mL of ice-cold water, a first time, and with 1 mL ice cold water, a second time. Then, the cells were resuspended in 50 μL of ice-cold water. Electroporation was done with 50 μL of cells and 10-100 ng of linear double-stranded-DNA product by using a Gene Pulser™ (BioRad) (600 Ω, 25 μFD, and 250 volts). After electroporation, cells were added to 1 mL LB media incubated 1 h at 37° C., and finally spread onto LB-agar containing 25 mg/L of chloramphenicol or 50 mg/L of kanamycin to select antibiotic resistant transformants. The selected mutants were verified by PCR with primers upstream and downstream of the modified region and were grown in LB-agar at 42° C. for the loss of the helper plasmid. The mutants were tested for ampicillin sensitivity. The linear ds-DNA amplicons were obtained by PCR using pKD3, pKD4 and their derivates as template. The primers used had a part of the sequence complementary to the template and another part complementary to the side on the chromosomal DNA where the recombination must take place. For the genomic knock-out, the region of homology was designed 50-nt upstream and 50-nt downstream of the start and stop codon of the gene of interest. For the genomic knock-in, the transcriptional starting point (+1) had to be respected. PCR products were PCR-purified, digested with Dpnl, re-purified from an agarose gel, and suspended in elution buffer (5 mM Tris, pH 8.0). Selected mutants were transformed with pCP20 plasmid, which is an ampicillin and chloramphenicol resistant plasmid that shows temperature-sensitive replication and thermal induction of FLP synthesis. The ampicillin-resistant transformants were selected at 30° C., after which a few were colony purified in LB at 42° C. and then tested for loss of all antibiotic resistance and of the FLP helper plasmid. The gene knock outs and knock ins are checked with control primers.

In an example for sialic acid production, the mutant strain was derived from E. coli K12 MG1655 comprising genomic knock-ins of constitutive transcriptional units containing one or more copies of a glucosamine 6-phosphate N-acetyltransferase like e.g. GNA1 from Saccharomyces cerevisiae (UniProt ID P43577), an N-acylglucosamine 2-epimerase like e.g. AGE from Bacteroides ovatus (UniProt ID A7LVG6) and an N-acetylneuraminate synthase like e.g. NeuB from Neisseria meningitidis (UniProt ID E0NCD4) or NeuB Campylobacter jejuni (UniProt ID Q93MP9). Alternatively, and/or additionally, sialic acid production can be obtained by genomic knock-ins of constitutive transcriptional units containing a UDP-N-acetylglucosamine 2-epimerase like e.g. NeuC from C. jejuni (UniProt ID Q93MP8) and an N-acetylneuraminate synthase like e.g. NeuB from Neisseria meningitidis (UniProt ID E0NCD4) or NeuB from Campylobacter jejuni (UniProt ID Q93MP9). Alternatively and/or additionally, sialic acid production can be obtained by genomic knock-ins of constitutive transcriptional units containing a phosphoglucosamine mutase like e.g. glmM from E. coli (UniProt ID P31120), an N-acetylglucosamine-1-phosphate uridyltransferase/glucosamine-1-phosphate acetyltransferase like e.g. glmU from E. coli (UniProt ID P0ACC7), a UDP-N-acetylglucosamine 2-epimerase like e.g. NeuC from C. jejuni (UniProt ID Q93MP8) and an N-acetylneuraminate synthase like e.g. NeuB from Neisseria meningitidis (UniProt ID E0NCD4) or NeuB from Campylobacter jejuni (UniProt ID Q93MP9). Alternatively, and/or additionally, sialic acid production can be obtained by genomic knock-ins of constitutive transcriptional units containing a bifunctional UDP-GlcNAc 2-epimerase/N-acetylmannosamine kinase like e.g. GNE from Mus musculus (strain C57BL/6J) (UniProt ID Q91WG8), an N-acylneuraminate-9-phosphate synthetase like e.g. NANS from Pseudomonas sp. UW4 (UniProt ID K9NPH9) and an N-acylneuraminate-9-phosphatase like e.g. NANP from Candidatus magnetomorum sp. HK-1 (UniProt ID KPA15328.1) or NANP from Bacteroides thetaiotaomicron (UniProt ID Q8A712). Alternatively, and/or additionally, sialic acid production can be obtained by genomic knock-ins of constitutive transcriptional units containing a phosphoglucosamine mutase like e.g. glmM from E. coli (UniProt ID P31120), an N-acetylglucosamine-1-phosphate uridyltransferase/glucosamine-1-phosphate acetyltransferase like e.g. glmU from E. coli (UniProt ID P0ACC7), a bifunctional UDP-GlcNAc 2-epimerase/N-acetylmannosamine kinase like e.g. GNE from M. musculus (strain C57BL/6J) (UniProt ID Q91WG8), an N-acylneuraminate-9-phosphate synthetase like e.g. NANS from Pseudomonas sp. UW4 (UniProt ID K9NPH9) and an N-acylneuraminate-9-phosphatase like e.g. NANP from Candidatus magnetomorum sp. HK-1 (UniProt ID KPA15328.1) or NANP from Bacteroides thetaiotaomicron (UniProt ID Q8A712).

Sialic acid production can further be optimized in the modified E. coli strain with genomic knock-outs of the E. coli genes comprising any one or more of nagA, nagB, nagC, nagD, nagE, nanA, nanE, nanK, manX, manY and manZ as described in WO18122225, and/or genomic knock-outs of the E. coli genes comprising any one or more of nanT, poxB, ldhA, adhE, aldB, pf/A, pflC, ybiY, ackA and/or pta and with genomic knock-ins of constitutive transcriptional units comprising one or more copies of an L-glutamine-D-fructose-6-phosphate aminotransferase like e.g. the mutant glmS*54 from E. coli (differing from the wild-type E. coli glmS, having UniProt ID P17169, by an A39T, an R250C and an G472S mutation as described by Deng et al. (Biochimie 88, 419-29 (2006)) and an acetyl-CoA synthetase like e.g. acs from E. coli (UniProt ID P27550).

In an example for sialylated oligosaccharide production like e.g. LSTc (Neu5Ac-α2,6-Gal-β1,4-GlcNAc-β1,3-Gal-β1,4-Glc), said sialic acid production strains were further modified to express an N-acylneuraminate cytidylyltransferase like e.g. the NeuA enzyme from Pasteurella multocida (SEQ ID NO 02) and to express one or more copies of a beta-galactoside alpha-2,6-sialyltransferase like e.g. PdST6 from Photobacterium damselae (UniProt ID O66375) or a PdST6-like polypeptide consisting of amino acid residues 108 to 497 of UniProt ID O66375 having beta-galactoside alpha-2,6-sialyltransferase activity or the polypeptide with SEQ ID NO 01. Constitutive transcriptional units of the N-acylneuraminate cytidylyltransferase and the sialyltransferase(s) can be delivered to the modified strain either via genomic knock-in or via expression plasmids. The strains were additionally modified with genomic knock-outs of the E. coli LacZ, LacY and LacA genes and with a genomic knock-in of a constitutive transcriptional unit for a lactose permease like e.g. E. coli LacY (UniProt ID P02920). All modified strains producing sialic acid, CMP-sialic acid and/or sialylated oligosaccharides could optionally be adapted for growth on sucrose via genomic knock-ins of constitutive transcriptional units containing a sucrose transporter like e.g. CscB from E. coli W (UniProt ID E0IXR1), a fructose kinase like e.g. Frk originating from Z. mobilis (UniProt ID Q03417) and a sucrose phosphorylase like e.g. BaSP from B. adolescentis (UniProt ID A0ZZH6).

In an example to produce lacto-N-triose (LN3, GlcNAc-β1,3-Gal-β1,4-Glc), the modified strain was derived from E. coli K12 MG1655 and modified with a knock-out of the E. coli lacZ, lacY, lacA and nagB genes and with genomic knock-ins of constitutive transcriptional units for a lactose permease like e.g. the E. coli LacY (UniProt ID P02920) and a galactoside beta-1,3-N-acetylglucosaminyltransferase like e.g. IgtA (UniProt ID Q9JXQ6) from N. meningitidis. In an example for production of LN3 derived oligosaccharides like lacto-N-neotetraose (LNnT, Gal-β1,4-GlcNAc-β1,3-Gal-β1,4-Glc), the modified LN3 producing strain was further modified with a constitutive transcriptional unit delivered to the strain either via genomic knock-in or from an expression plasmid for an N-acetylglucosamine beta-1,4-galactosyltransferase like e.g. LgtB (Uniprot ID Q51116) from Neisseria meningitidis.

LN3 and/or LNnT production can further be optimized in the modified E. coli strains with genomic knock-outs of the E. coli genes comprising any one or more of galT, ushA, ldhA and agp. The modified LN3 and/or LNnT producing strains can also be optionally modified for enhanced UDP-GlcNAc production with a genomic knock-in of a constitutive transcriptional unit for an L-glutamine-D-fructose-6-phosphate aminotransferase like e.g. the mutant glmS*54 from E. coli (differing from the wild-type E. coli glmS protein, having UniProt ID P17169, by an A39T, an R250C and an G472S mutation as described by Deng et al. (Biochimie 2006, 88: 419-429). The modified E. coli strains can also optionally be adapted with a genomic knock-in of a constitutive transcriptional unit for a UDP-glucose-4-epimerase like e.g. galE from E. coli (UniProt ID P09147), a phosphoglucosamine mutase like e.g. glmM from E. coli (UniProt ID P31120) and an N-acetylglucosamine-1-phosphate uridylyltransferase/glucosamine-1-phosphate acetyltransferase like e.g. glmU from E. coli (UniProt ID P0ACC7).

Preferably but not necessarily, any one or more of the glycosyltransferases and/or the proteins involved in nucleotide-activated sugar synthesis were N- and/or C-terminally fused to a solubility enhancer tag like e.g. a SUMO-tag, an MBP-tag, His, FLAG, Strep-II, Halo-tag, NusA, thioredoxin, GST and/or the Fh8-tag to enhance their solubility (Costa et al., Front. Microbiol. 2014, https://doi.org/10.3389/fmicb.2014.00063; Fox et al., Protein Sci. 2001, 10(3), 622-630; Jia and Jeaon, Open Biol. 2016, 6: 160196).

Optionally, the modified E. coli strains were modified with a genomic knock-ins of a constitutive transcriptional unit encoding a chaperone protein like e.g. DnaK, DnaJ, GrpE or the GroEL/ES chaperonin system (Baneyx F., Palumbo J. L. (2003) Improving Heterologous Protein Folding via Molecular Chaperone and Foldase Co-Expression. In: Vaillancourt P. E. (eds) E. coli Gene Expression Protocols. Methods in Molecular Biology™, vol 205. Humana Press).

All constitutive promoters, UTRs and terminator sequences originated from the libraries described by Cambray et al. (Nucleic Acids Res. 2013, 41(9), 5139-5148), Dunn et al. (Nucleic Acids Res. 1980, 8, 2119-2132), Edens et al. (Nucleic Acids Res. 1975, 2, 1811-1820), Kim and Lee (FEBS Letters 1997, 407, 353-356) and Mutalik et al. (Nat. Methods 2013, No. 10, 354-360).

The proteins described in present invention are summarized in Table 1.

All genes were ordered synthetically atTwist Bioscience (twistbioscience.com) or IDT (eu.idtdna.com) and the codon usage was adapted using the tools of the supplier.

All strains were stored in cryovials at −80° C. (overnight LB culture mixed in a 1:1 ratio with 70% glycerol).

### Cultivation Conditions

A preculture of 96-well microtiter plate experiments was started from a cryovial, in 150 μL LB and was incubated overnight at 37° C. on an orbital shaker at 800 rpm. This culture was used as inoculum for a 96 well square microtiter plate, with 400 μL minimal medium by diluting 400×. These final 96-well culture plates were then incubated at 37° C. on an orbital shaker at 800 rpm for 72 h, or shorter, or longer. To measure sugar concentrations at the end of the cultivation experiment whole broth samples were taken from each well by boiling the culture broth for 15 min at 60° C. before spinning down the cells (=average of intra- and extracellular sugar concentrations).

A preculture for the bioreactor was started from an entire 1 mL cryovial of a certain strain, inoculated in 250 mL or 500 mL minimal medium in a 1 L or 2.5 L shake flask and incubated for 24 h at 37° C. on an orbital shaker at 200 rpm. A 5 L bioreactor was then inoculated (250 mL inoculum in 2 L batch medium); the process was controlled by MFCS control software (Sartorius Stedim Biotech, Melsungen, Germany). Culturing condition were set to 37° C., and maximal stirring; pressure gas flow rates were dependent on the strain and bioreactor. The pH was controlled at 6.8 using 0.5 M H2S04 and 20% NH4OH. The exhaust gas was cooled. 10% solution of silicone antifoaming agent was added when foaming raised during the fermentation.

### Example 3. Production of LSTc with a Modified E. coli Host

An E. coli K-12 MG1655 strain modified for production of sialic acid, comprising genomic knock-ins of constitutive transcriptional units containing the UDP-N-acetylglucosamine 2-epimerase NeuC from C. jejuni (UniProt ID Q93MP8) and the N-acetylneuraminate synthase NeuB from Neisseria meningitidis (UniProt ID E0NCD4), and modified for growth on sucrose as described in Example 2 was further modified with a genomic knock-in of a constitutive transcriptional unit for the galactoside beta-1,3-N-acetylglucosaminyltransferase LgtA from N. meningitidis (UniProt ID Q9JXQ6) and for the N-acetylglucosamine beta-1,4-galactosyltransferase LgtB from N. meningitidis (UniProt ID Q51116) to allow production of lacto-N-neotetraose (LNnT, Gal-β1,4-GlcNAc-β1,3-Gal-β1,4-Glc). Optionally, multiple copies of the LgtA and/or LgtB genes could be added to the modified E. coli strain. In a next step, the novel strain was transformed with an expression plasmid containing a constitutive transcriptional unit for the N-acylneuraminate cytidylyltransferase neuA from P. multocida with SEQ ID NO 02 and for i) an alpha-2,6-sialyltransferase with SEQ ID NO 01 or ii) a PdST6-like polypeptide consisting of amino acid residues 108 to 497 of UniProt ID O66375 having beta-galactoside alpha-2,6-sialyltransferase activity acting as a reference alpha-2,6-sialyltransferase. The novel strains were evaluated in a growth experiment for production of LSTc (Neu5Ac-α2,6-Gal-β1,4-GlcNAc-β1,3-Gal-β1,4-Glc) according to the culture conditions provided in Example 2, in which the strains were cultivated in minimal medium with 30 g/L sucrose and 20 g/L lactose. The strains were grown in four biological replicates in a 96-well plate. After 72 h of incubation, the culture broth was harvested, and the sugars were analysed on UPLC. The measured LSTc concentration and maximum growth speed (mumax) measured of strain B, expressing an alpha-2,6-sialyltransferase with SEQ ID NO 01, was averaged over all biological replicates and then normalized to the averaged LSTc concentration or maximum growth speed, respectively, of a reference strain A expressing a PdST6-like polypeptide consisting of amino acid residues 108 to 497 of UniProt ID O66375 having beta-galactoside alpha-2,6-sialyltransferase activity. The experiment showed both strains A and B, each expressing a different alpha-2,6-sialyltransferase, were able to produce LSTc. Surprisingly, and as demonstrated in Table 2, strain B expressing the sialyltransferase with SEQ ID NO 01 showed a production of LSTc that was six times higher than the production of LSTc measured in the reference strain A. Strain B further demonstrated to have a higher maximum growth speed compared to the reference strain A.

TABLE 2
Relative production of LSTc (%) in and relative maximum growth
speed (%) of a modified E. coli strain expressing an alpha-
2,6-sialyltransferase with SEQ ID NO 01 and compared to a reference
strain expressing a PdST6-like polypeptide consisting of amino
acid residues 108 to 497 of UniProt ID O66375, when evaluated
in a growth experiment according to the culture conditions
provided in Example 2, in which the culture medium contained
30 g/L sucrose and 20 g/L lactose.

| | Alpha-2,6-sialyltransferase | | |
|---|---|---|---|
| Strain | expressed | LSTc (%) | mumax (%) |
| A | PdST6-like polypeptide | 100 | 100 |
| | consisting of amino acid | | |
| | residues 108 to 497 of | | |
| | UniProt ID O66375 | | |
| B | SEQ ID NO 01 | 615 | 114 |

### Example 4. Production of LSTc with a Modified E. coli Host

Both modified E. coli strains A and B as described in Example 3 were evaluated in another growth experiment for production of LNnT, LSTc (Neu5Ac-α2,6-Gal-1,4-GlcNAc-β1,3-Gal-β1,4-Glc) and 6′SL (Neu5Ac-α2,6-Gal-β1,4-Glc) according to the culture conditions provided in Example 2, in which the strains were cultivated in minimal medium with 30 g/L sucrose and 20 g/L lactose. The strains were grown in four biological replicates in a 96-well plate. After 72 h of incubation, the culture broth was harvested, and the sugars were analysed on UPLC. The measured LNnT, LSTc and 6′SL concentration measured of strain B, expressing an alpha-2,6-sialyltransferase with SEQ ID NO 01, was averaged over all biological replicates and then normalized to the averaged LNnT, LSTc and 6′SL concentration, respectively, of the reference strain A expressing a PdST6-like polypeptide consisting of amino acid residues 108 to 497 of UniProt ID O66375 having beta-galactoside alpha-2,6-sialyltransferase activity. The experiment showed both strains A and B, each expressing a different alpha-2,6-sialyltransferase, were able to produce LNnT, LSTc and 6′SL. As demonstrated in Table 3, the sialyltransferase with SEQ ID NO 01 had a better 6-sialyltransferase binding activity on LNnT than the reference sialyltransferase, with strain B showing a production of LSTc that was six times higher than the production of LSTc measured in the reference strain A and having three times less of non-sialylated LNnT compared to strain A. In addition, the reference sialyltransferase (i.e. the PdST6-like polypeptide consisting of amino acid residues 108 to 497 of UniProt ID O66375) had a better 6-sialyltransferase binding activity on lactose than the sialyltransferase with SEQ ID NO 01, with the reference strain A showing a production of 6′SL that was 2.5 times higher than the production of 6′SL measured in strain B.

TABLE 3
Relative production of LSTc (%), LNnT (%) and 6′SL
(%) in a modified E. coli strain expressing an
alpha-2,6-sialyltransferase with SEQ ID NO 01 and compared
to a reference strain expressing a PdST6-like polypeptide
consisting of amino acid residues 108 to 497 of UniProt
ID O66375, when evaluated in a growth experiment according
to the culture conditions provided in Example 2, in which the
culture medium contained 30 g/L sucrose and 20 g/L lactose.

| | Alpha-2,6-sialyltransferase | | | |
|---|---|---|---|---|
| Strain | expressed | LSTc (%) | LNnT (%) | 6′SL (%) |
| A | PdST6-like polypeptide | 100 | 100 | 100 |
| | consisting of amino acid | | | |
| | residues 108 to 497 of | | | |
| | UniProt ID O66375 | | | |
| B | SEQ ID NO 01 | 615 | 31 | 40 |

### Example 5. Production of LSTc with a Modified E. coli Host

An E. coli K-12 MG1655 strain modified to produce LNnT as described in Example 2 is further transformed with an expression plasmid containing a constitutive transcriptional unit for the N-acylneuraminate cytidylyltransferase neuA from P. multocida with SEQ ID NO 02 and for i) an alpha-2,6-sialyltransferase with SEQ ID NO 01 or ii) a PdST6-like polypeptide consisting of amino acid residues 108 to 497 of UniProt ID O66375 having beta-galactoside alpha-2,6-sialyltransferase activity acting as a reference alpha-2,6-sialyltransferase. The novel strains are evaluated in a growth experiment for production of LSTc (Neu5Ac-α2,6-Gal-β1,4-GlcNAc-β1,3-Gal-β1,4-Glc) according to the culture conditions provided in Example 2, in which the strains are cultivated in minimal medium with glycerol as carbon source and sialic acid and lactose added to the medium.

### Example 6. Production of LSTc with a Modified E. coli Host

An E. coli K-12 MG1655 strain modified to produce LNnT as described in Example 2 is further modified for growth on sucrose as described in Example 2. Next, the strain is transformed with an expression plasmid containing a constitutive transcriptional unit for the N-acylneuraminate cytidylyltransferase neuA from P. multocida with SEQ ID NO 02 and for i) an alpha-2,6-sialyltransferase with SEQ ID NO 01 or ii) a PdST6-like polypeptide consisting of amino acid residues 108 to 497 of UniProt ID O66375 having beta-galactoside alpha-2,6-sialyltransferase activity acting as a reference alpha-2,6-sialyltransferase. The novel strains are evaluated in a growth experiment for production of LSTc (Neu5Ac-α2,6-Gal-β1,4-GlcNAc-β1,3-Gal-β1,4-Glc) according to the culture conditions provided in Example 2, in which the strains are cultivated in minimal medium with sucrose as carbon source and sialic acid and lactose added to the medium.

### Example 7. Production of LSTc with Modified E. coli Hosts when Evaluated in a Fed-Batch Fermentation Process with Sucrose and Lactose

The modified E. coli strain expressing the alpha-2,6-sialyltransferase with SEQ ID NO 01 and able to produce LSTc as described in Example 3 was selected for further evaluation in a fed-batch fermentation process. Fed-batch fermentations at bioreactor scale were performed as described in Example 1. Sucrose was used as a carbon source and lactose was added in the batch medium. During fed-batch, sucrose was added via an additional feed. In contrast to the cultivation experiments that are described herein and wherein only end samples were taken at the end of cultivation (i.e. 72 hours as described herein), regular broth samples were taken at several time points during the fermentation process and sugars produced were measured using UPLC as described in Example 1.

### Example 8. Evaluation of Alpha-2,6-Sialyltransferase Activity In Vitro

Another example provides the evaluation of alpha-2,6-sialyltransferase activity of the enzyme with SEQ ID NO 01 of the present invention in an in vitro enzymatic assay. Said enzyme can be produced in a cell-free expression system such as but not limited to the PURExpress system (NEB), or in a host organism such as but not limited to Escherichia coli or Saccharomyces cerevisiae, after which the enzyme can be isolated and optionally further purified. The enzyme extract or purified enzyme is added to a reaction mixture together with CMP-sialic acid and a buffering component such as Tris-HCl or HEPES and a substrate like e.g. lacto-N-neotetraose (LNnT) or lactose. Said reaction mixture is then incubated at a certain temperature (for example 37° C.) for a certain amount of time (for example 8 hours, 16 hours, 24 hours), during which the LNnT or lactose will be converted by the enzyme using CMP-sialic acid to LSTc or 6′SL, respectively. The oligosaccharides are then separated from the reaction mixture by methods known in the art. Further purification of LSTc or 6′SL can be performed if preferred. At the end of the reaction or after separation and/or purification, the production of LSTc or 6′SL is measured via analytical methods as described in Example 1 and known by the person skilled in the art.

### Example 9. Materials and Methods Saccharomyces cerevisiae

### Media

Strains were grown on Synthetic Defined yeast medium with Complete Supplement Mixture (SD CSM) or CSM drop-out (SD CSM-Ura, SD CSM-Trp, SD CSM-His) containing 6.7 g/L Yeast Nitrogen Base without amino acids (YNB w/o AA, Difco), 20 g/L agar (Difco) (solid cultures), 22 g/L glucose monohydrate or 20 g/L lactose and 0.79 g/L CSM or 0.77 g/L CSM-Ura, 0.77 g/L CSM-Trp, or 0.77 g/L CSM-His (MP Biomedicals).

### Strains

S. cerevisiae BY4742 created by Brachmann et al. (Yeast (1998) 14:115-32) was used, available in the Euroscarf culture collection. All mutant strains were created by homologous recombination or plasmid transformation using the method of Gietz (Yeast 11:355-360, 1995).

### Plasmids

In an example to produce sialic acid and CMP-sialic acid, a yeast expression plasmid was derived from the pRS420-plasmid series (Christianson et al., 1992, Gene 110: 119-122) containing the TRP1 selection marker and constitutive transcriptional units for an L-glutamine-D-fructose-6-phosphate aminotransferase like e.g. the mutant glmS*54 from E. coli (differing from the wild-type E. coli glmS, having UniProt ID P17169, by an A39T, an R250C and an G472S mutation as described by Deng et al. (Biochimie 88, 419-29 (2006)), a phosphatase like e.g. SurE from E. coli (UniProt ID P0A840), an N-acetylglucosamine 2-epimerase like e.g. AGE from B. ovatus (UniProt ID A7LVG6), an N-acetylneuraminate synthase like e.g. NeuB from N. meningitidis (UniProt ID E0NCD4) and an N-acylneuraminate cytidylyltransferase like e.g. NeuA from P. multocida with SEQ ID NO 02. Optionally, a constitutive transcriptional unit for a glucosamine 6-phosphate N-acetyltransferase like e.g. GNA1 from S. cerevisiae (UniProt ID P43577) was added as well. In an example to produce sialylated oligosaccharides, the plasmid further comprised constitutive transcriptional units for a lactose permease like e.g. LAC12 from K. lactis (UniProt ID P07921), and a beta-galactoside alpha-2,6-sialyltransferase like e.g. PdST6 from Photobacterium damselae (UniProt ID O66375) or a PdST6-like polypeptide consisting of amino acid residues 108 to 497 of UniProt ID O66375 having beta-galactoside alpha-2,6-sialyltransferase activity or the polypeptide with SEQ ID NO 01.

In one example to produce UDP-galactose, a yeast expression plasmid can be derived from the pRS420-plasmid series (Christianson et al., 1992, Gene 110: 119-122) containing the HIS3 selection marker and a constitutive transcriptional unit for a UDP-glucose-4-epimerase like e.g. galE from E. coli (UniProt ID P09147). This plasmid can be further modified with constitutive transcriptional units for a lactose permease like e.g. LAC12 from K. lactis (UniProt ID P07921) and a galactoside beta-1,3-N-acetylglucosaminyltransferase activity like e.g. IgtA from N. meningitidis (UniProt ID Q9JXQ6) to produce LN3. In an example for production of LN3 derived oligosaccharides like lacto-N-neotetraose (LNnT, Gal-β1,4-GlcNAc-β1,3-Gal-β1,4-Glc), the modified LN3 producing strain was further modified with a constitutive transcriptional unit for an N-acetylglucosamine beta-1,4-galactosyltransferase like e.g. LgtB (Uniprot ID Q51116) from Neisseria meningitidis.

Preferably but not necessarily, any one or more of the glycosyltransferases and/or the proteins involved in nucleotide-activated sugar synthesis were N- and/or C-terminally fused to a SUMOstar tag (e.g. obtained from pYSUMOstar, Life Sensors, Malvern, PA) to enhance their solubility.

Optionally, the mutant yeast strains were modified with a genomic knock-in of a constitutive transcriptional unit encoding a chaperone protein like e.g. Hsp31, Hsp32, Hsp33, Sno4, Kar2, Ssb1, Sse1, Sse2, Ssa1, Ssa2, Ssa3, Ssa4, Ssb2, Ecm10, Ssc1, Ssq1, Ssz1, Lhs1, Hsp82, Hsc82, Hsp78, Hsp104, Tcp1, Cct4, Cct8, Cct2, Cct3, Cct5, Cct6 or Cct7 (Gong et al., 2009, Mol. Syst. Biol. 5: 275). Plasmids were maintained in the host E. coli DH5alpha (F⁻, phi80dlacZdeltaM15, delta(lacZYA-argF)U169, deoR, recA1, endA1, hsdR17(rk⁻, mk⁺), phoA, supE44, lambda-, thi-1, gyrA96, re/A1) bought from Invitrogen.

### Heterologous and Homologous Expression

Genes that needed to be expressed, be it from a plasmid or from the genome were synthetically synthetized with one of the following companies: DNA2.0, Gen9, IDT or Twist Bioscience. Expression could be further facilitated by optimizing the codon usage to the codon usage of the expression host. Genes were optimized using the tools of the supplier.

### Cultivations Conditions

In general, yeast strains were initially grown on SD CSM plates to obtain single colonies. These plates were grown for 2-3 days at 30° C. Starting from a single colony, a preculture was grown over night in 5 mL at 30° C., shaking at 200 rpm. Subsequent 125 mL shake flask experiments were inoculated with 2% of this preculture, in 25 mL media. These shake flasks were incubated at 30° C. with an orbital shaking of 200 rpm.

### Gene Expression Promoters

Genes were expressed using synthetic constitutive promoters, as described by e.g. Blazeck (Biotechnology and Bioengineering, Vol. 109, No. 11, 2012), Redden and Alper (Nat. Commun. 2015, 6, 7810), Liu et al. (Microb. Cell Fact. 2020, 19, 38), Xu et al. (Microb. Cell Fact. 2021, 20, 148) and Lee et al. (ACS Synth. Biol. 2015, 4(9), 975-986).

### Example 10. Production of LSTc with a Modified S. cerevisiae Host

A S. cerevisiae strain is modified for production of CMP-sialic acid and LNnT and for expression of an alpha-2,6-sialyltransferase as described in Example 9 with a first yeast expression plasmid comprising constitutive transcriptional units for LAC12 from K. lactis (UniProt ID P07921), the mutant glmS*54 from E. coli (differing from the wild-type E. coli glmS, having UniProt ID P17169, by an A39T, an R250C and an G472S mutation as described by Deng et al. (Biochimie 88, 419-29 (2006)), the phosphatase SurE from E. coli (UniProt ID P0A840), AGE from B. ovatus (UniProt ID A7LVG6), NeuB from N. meningitidis (UniProt ID E0NCD4), NeuA from P. multocida with SEQ ID NO 02, and i) a PdST6-like polypeptide consisting of amino acid residues 108 to 497 of UniProt ID O66375 having beta-galactoside alpha-2,6-sialyltransferase activity or ii) a polypeptide with SEQ ID NO 01, and a second yeast expression plasmid comprising constitutive transcriptional units for galE from E. coli (UniProt ID P09147), LgtA from N. meningitidis (UniProt ID Q9JXQ6) and LgtB from N. meningitidis (UniProt ID Q51116). The novel strains are evaluated for production of LSTc when evaluated in a 3-days growth experiment according to the culture conditions provided in Example 9 using appropriate selective medium comprising lactose.

### Example 11. Materials and Methods Bacillus subtilis

### Media

Two media are used to cultivate B. subtilis: i.e. a rich Luria Broth (LB) and a minimal medium for shake flask cultures. The LB medium consisted of 1% tryptone peptone (Difco), 0.5% yeast extract (Difco) and 0.5% sodium chloride (VWR). Luria Broth agar (LBA) plates consisted of the LB media, with 12 g/L agar (Difco) added. The minimal medium contained 2.00 g/L (NH₄)₂SO₄, 7.5 g/L KH₂PO₄, 17.5 g/L K₂HPO₄, 1.25 g/L Na-citrate, 0.25 g/L MgSO₄·7H₂O, 0.05 g/L tryptophan, from 10 up to 30 g/L glucose (or another carbon source including but not limited to fructose, maltose, sucrose, glycerol and maltotriose), 10 mL/L trace element mix and 10 mL/L Fe-citrate solution. The medium was set to a pH of 7.0 with 1 M KOH. Depending on the experiment lactose is added as a precursor. The trace element mix consisted of 0.735 g/L CaCl₂·2H₂O, 0.1 g/L MnCl₂·2H₂O, 0.033 g/L CuCl₂·2H₂O, 0.06 g/L CoCl₂·6H₂O, 0.17 g/L ZnCl₂, 0.0311 g/L H₃BO₄, 0.4 g/L Na₂EDTA·2H₂O and 0.06 g/L Na₂MoO₄. The Fe-citrate solution contained 0.135 g/L FeCl₃·6H₂O, 1 g/L Na-citrate (Hoch 1973 PMC1212887).

Complex medium, e.g. LB, was sterilized by autoclaving (121° C., 21 min) and minimal medium by filtration (0.22 μm Sartorius). When necessary, the medium was made selective by adding an antibiotic (e.g. zeocin (20 mg/L)).

### Strains, Plasmids and Mutations

Bacillus subtilis 168 is used as available at the Bacillus Genetic Stock Center (Ohio, USA).

Plasmids for gene deletion via Cre/lox are constructed as described by Yan et al. (Appl & Environm microbial, September 2008, p 5556-5562). Gene disruption is done via homologous recombination with linear DNA and transformation via the electroporation as described by Xue et al. (J. microb. Meth. 34 (1999) 183-191). The method of gene knockouts is described by Liu et al. (Metab. Engine. 24 (2014) 61-69). This method uses 1000 bp homologies up- and downstream of the target gene.

Integrative vectors as described by Popp et al. (Sci. Rep., 2017, 7, 15158) are used as expression vector and could be further used for genomic integrations if necessary. A suitable promoter for expression can be derived from the part repository (iGem): sequence id: BBa_K143012, BBa_K823000, BBa_K823002 or BBa_K823003. Cloning can be performed using Gibson Assembly, Golden Gate assembly, Cliva assembly, LCR or restriction ligation.

In an example for sialic acid (Neu5Ac) production, the engineered strain was derived from B. subtilis comprising knockouts of the B. subtilis nagA, nagB and gamA genes and genomic knock-ins of constitutive transcriptional units containing a phosphoglucosamine mutase like e.g. glmM from E. coli (UniProt ID P31120), an N-acetylglucosamine-1-phosphate uridyltransferase/glucosamine-1-phosphate acetyltransferase like e.g. glmU from E. coli (UniProt ID P0ACC7), a UDP-N-acetylglucosamine 2-epimerase like e.g. neuC from C. jejuni (UniProt ID Q93MP8) and an N-acetylneuraminate synthase like e.g. neuB from N. meningitidis (UniProt ID E0NCD4). Sialic acid production can also be obtained in modified B. subtilis comprising knockouts of the B. subtilis nagA, nagB and gamA genes and genomic knock-ins of constitutive transcriptional units containing an N-acetylglucosamine 2-epimerase like e.g. AGE from B. ovatus (UniProt ID A7LVG6) and an N-acetylneuraminate synthase like e.g. NeuB from N. meningitidis (UniProt ID E0NCD4). To enhance the intracellular glucosamine-6-phosphate pool, the modified strain can further be modified with a genomic knock-in of one or more constitutive transcriptional units containing a glutamine-fructose-6-P-aminotransferase like e.g. the native glutamine-fructose-6-P-aminotransferase glmS (UniProt ID P0C173). Optionally, the strains were also modified for expression of a phosphatase like e.g. SurE from E. coli (UniProt ID P0A840). In an example for sialylated oligosaccharide production, the sialic acid production strains further need to express an N-acylneuraminate cytidylyltransferase like e.g. neuA from P. multocida with SEQ ID NO 02, and a beta-galactoside alpha-2,6-sialyltransferase like e.g. PdST6 from Photobacterium damselae (UniProt ID O66375) or a PdST6-like polypeptide consisting of amino acid residues 108 to 497 of UniProt ID O66375 having beta-galactoside alpha-2,6-sialyltransferase activity or the polypeptide with SEQ ID NO 01. Constitutive transcriptional units of the N-acylneuraminate cytidylyltransferase and the sialyltransferases can be delivered to the engineered strain either via genomic knock-in or via expression plasmids. If the engineered strains producing sialic acid and CMP-sialic acid were intended to make sialylated lactose structures, the strains were additionally modified with a genomic knock-in of a constitutive transcriptional unit for a lactose permease like e.g. the E. coli LacY (UniProt ID P02920).

In an example for LN3 production, the engineered strain was derived from B. subtilis comprising knockouts of the B. subtilis nagB and gamA genes and genomic knock-ins of constitutive transcriptional units containing a galactoside beta-1,3-N-acetylglucosaminyltransferase like e.g. IgtA from N. meningitidis (UniProt ID Q9JXQ6) and a lactose permease like e.g. LacY from E. coli (UniProt ID P02920). For LNnT production, the LN3 producing strain was further transformed with constitutive transcriptional units for an N-acetylglucosamine beta-1,4-galactosyltransferase like e.g. IgtB from N. meningitidis (UniProt ID Q51116).

### Heterologous and Homologous Expression

Genes that needed to be expressed, be it from a plasmid or from the genome were synthetically synthetized with one of the following companies: DNA2.0, Gen9, Twist Biosciences or IDT.

Expression could be further facilitated by optimizing the codon usage to the codon usage of the expression host. Genes were optimized using the tools of the supplier.

### Cultivation Conditions

A preculture of 96-well microtiter plate experiments was started from a cryovial or a single colony from an LB plate, in 150 μL LB and was incubated overnight at 37° C. on an orbital shaker at 800 rpm. This culture was used as inoculum for a 96-well square microtiter plate, with 400 μL minimal medium by diluting 400×. Each strain was grown in multiple wells of the 96-well plate as biological replicates. These final 96-well culture plates were then incubated at 37° C. on an orbital shaker at 800 rpm for 72 h, or shorter, or longer. At the end of the cultivation experiment samples were taken from each well to measure the supernatant concentration (extracellular sugar concentrations, after 5 min. spinning down the cells), or by boiling the culture broth for 15 min at 90° C. or for 60 min at 60° C. before spinning down the cells (=whole broth concentration, intra- and extracellular sugar concentrations, as defined herein).

Also, a dilution of the cultures was made to measure the optical density at 600 nm. The cell performance index or CPI was determined by dividing the oligosaccharide concentrations by the biomass, in relative percentages compared to a reference strain. The biomass is empirically determined to be approximately ⅓rd of the optical density measured at 600 nm.

### Optical Density, pH and Analytical Analysis

The determination of the optical density and the pH of the bacterial cultures as well as the analytical analysis were performed as described in Example 1.

### Example 12. Production of LSTc with a Modified B. subtilis Host

A wild-type B. subtilis strain is first modified for production of LN3 with genomic knockouts of the B. subtilis genes nagB and gamA together with genomic knock-ins of constitutive transcriptional units for the lactose permease LacY from E. coli (UniProt ID P02920) and IgtA from N. meningitidis (UniProt ID Q9JXQ6). In a next step, the modified B. subtilis strain is modified for production of LNnT with a genomic knock-in of a constitutive transcriptional unit for IgtB from N. meningitidis (UniProt ID Q51116). In a further step, the modified strain is transformed with an expression plasmid comprising constitutive transcriptional units for the N-acylneuraminate cytidylyltransferase neuA from P. multocida (SEQ ID NO 02) and the alpha-2,6-sialyltransferase with SEQ ID NO 01. The novel strain is evaluated for production of LSTc when evaluated in a 3-days growth experiment according to the culture conditions provided in Example 11 using appropriate selective medium comprising lactose.

### Example 13. Materials and Methods Corynebacterium glutamicum

### Media

Two different media are used to cultivate C. glutamicum: i.e. a rich tryptone-yeast extract (TY) medium and a minimal medium. The TY medium consisted of 1.6% tryptone (Difco), 1% yeast extract (Difco) and 0.5% sodium chloride (VWR). TY agar (TYA) plates consisted of the TY media, with 12 g/L agar (Difco) added. The minimal medium for the shake flask experiments contained 20 g/L (NH₄)₂SO₄, 5 g/L urea, 1 g/L KH₂PO₄, 1 g/L K₂HPO₄, 0.25 g/L MgSO₄·7H₂O, 42 g/L MOPS, from 10 up to 30 g/L glucose (or another carbon source including but not limited to fructose, maltose, sucrose, glycerol and maltotriose) and 1 mL/L trace element mix. Depending on the experiment lactose is added as a precursor. The trace element mix consisted of 10 g/L CaCl₂, 10 g/L FeSO₄·7H₂O, 10 g/L MnSO₄·H₂O, 1 g/L ZnSO₄·7H₂O, 0.2 g/L CuSO₄, 0.02 g/L NiCI₂·6H₂O, 0.2 g/L biotin (pH 7.0) and 0.03 g/L protocatechuic acid.

Complex medium, e.g. TY, was sterilized by autoclaving (121° C., 21 min) and minimal medium by filtration (0.22 μm Sartorius). When necessary, the medium was made selective by adding an antibiotic (e.g. kanamycin, ampicillin).

### Strains and Mutations

Corynebacterium glutamicum ATCC 13032 was used as available at the American Type Culture Collection. Integrative plasmid vectors based on the Cre/loxP technique as described by Suzuki et al. (Appl. Microbiol. Biotechnol., 2005 April, 67(2):225-33) and temperature-sensitive shuttle vectors as described by Okibe et al. (J. Microbiol. Meth. 85, 2011, 155-163) are constructed for gene deletions, mutations and insertions. Suitable promoters for (heterologous) gene expression can be derived from Yim et al. (Biotechnol. Bioeng., 2013 November, 110(11):2959-69). Cloning can be performed using Gibson Assembly, Golden Gate assembly, Cliva assembly, LCR or restriction ligation.

In an example for sialic acid production, the engineered strain was derived from C. glutamicum comprising knockouts of the C. glutamicum ldh, cgl2645 and nagB genes and genomic knock-ins of constitutive transcriptional units containing a phosphoglucosamine mutase like e.g. glmM from E. coli (UniProt ID P31120), an N-acetylglucosamine-1-phosphate uridyltransferase/glucosamine-1-phosphate acetyltransferase like e.g. glmU from E. co/i (UniProt ID P0ACC7), a UDP-N-acetylglucosamine 2-epimerase like e.g. neuC from C. jejuni (UniProt ID Q93MP8) and an N-acetylneuraminate synthase like e.g. neuB from N. meningitidis (UniProt ID E0NCD4). To enhance the intracellular glucosamine-6-phosphate pool, the modified strain can further be modified with a genomic knock-in of one or more constitutive transcriptional units containing a glutamine-fructose-6-P-aminotransferase like e.g. the native glutamine-fructose-6-P-aminotransferase glmS (UniProt ID Q8NND3). In an example for sialylated oligosaccharide production, the sialic acid production strains further need to express an N-acylneuraminate cytidylyltransferase like e.g. neuA from P. multocida with SEQ ID NO 02, and a beta-galactoside alpha-2,6-sialyltransferase like e.g. PdST6 from Photobacterium damselae (UniProt ID 066375) or a PdST6-like polypeptide consisting of amino acid residues 108 to 497 of UniProt ID O66375 having beta-galactoside alpha-2,6-sialyltransferase activity or the polypeptide with SEQ ID NO 01. Constitutive transcriptional units of the N-acylneuraminate cytidylyltransferase and the sialyltransferases can be delivered to the engineered strain either via genomic knock-in or via expression plasmids. If the engineered strains producing sialic acid and CMP-sialic acid were intended to make sialylated lactose structures, the strains were additionally modified with a genomic knock-in of a constitutive transcriptional unit for a lactose permease like e.g. the E. coli LacY (UniProt ID P02920).

In an example for LN3 production, the engineered strain was derived from C. glutamicum comprising knockouts of the C. glutamicum ldh, cgl2645 and nagB genes and genomic knock-ins of constitutive transcriptional units containing a galactoside beta-1,3-N-acetylglucosaminyltransferase like e.g. IgtA from N. meningitidis (UniProt ID Q9JXQ6) and a lactose permease like e.g. LacY from E. coli (UniProt ID P02920). In an example for LNnT production, the LN3 producing strain was further transformed with a constitutive transcriptional unit for an N-acetylglucosamine beta-1,4-galactosyltransferase like e.g. IgtB from N. meningitidis (UniProt ID Q51116).

### Heterologous and Homologous Expression

Genes that needed to be expressed, be it from a plasmid or from the genome were synthetically synthetized with one of the following companies: DNA2.0, Gen9, Twist Biosciences or IDT.

Expression could be further facilitated by optimizing the codon usage to the codon usage of the expression host. Genes were optimized using the tools of the supplier.

### Cultivation Conditions

A preculture of 96-well microtiter plate experiments was started from a cryovial or a single colony from a TY plate, in 150 μL TY and was incubated overnight at 37° C. on an orbital shaker at 800 rpm. This culture was used as inoculum for a 96-well square microtiter plate, with 400 μL minimal medium by diluting 400×. Each strain was grown in multiple wells of the 96-well plate as biological replicates. These final 96-well culture plates were then incubated at 37° C. on an orbital shaker at 800 rpm for 72 h, or shorter, or longer. At the end of the cultivation experiment samples were taken from each well to measure the supernatant concentration (extracellular sugar concentrations, after 5 min. spinning down the cells), or by boiling the culture broth for 15 min at 60° C. before spinning down the cells (=whole broth concentration, intra- and extracellular sugar concentrations, as defined herein).

Also, a dilution of the cultures was made to measure the optical density at 600 nm. The cell performance index or CPI was determined by dividing the oligosaccharide concentrations, e.g. sialyllactose concentrations, measured in the whole broth by the biomass, in relative percentages compared to the reference strain. The biomass is empirically determined to be approximately ⅓rd of the optical density measured at 600 nm.

### Optical Density, pH and Analytical Analysis

The determination of the optical density and the pH of the bacterial cultures as well as the analytical analysis were performed as described in Example 1.

### Example 14. Production of LSTc with a Modified C. glutamicum Host

A wild-type C. glutamicum strain is first modified for production of LN3 with genomic knockouts of the ldh, cgl2645 and nagB genes together with genomic knock-ins of constitutive transcriptional units for the lactose permease LacY from E. coli (UniProt ID P02920) and IgtA from N. meningitidis (UniProt ID Q9JXQ6). In a next step, the modified C. glutamicum strain is modified for production of LNnT with a genomic knock-in of a constitutive transcriptional unit for IgtB from N. meningitidis (UniProt ID Q51116). In a further step, the modified strain is transformed with an expression plasmid comprising constitutive transcriptional units for the N-acylneuraminate cytidylyltransferase neuA from P. multocida (SEQ ID NO 02) and the alpha-2,6-sialyltransferase with SEQ ID NO 01. The novel strain is evaluated for production of LSTc when evaluated in a 3-days growth experiment according to the culture conditions provided in Example 13 using appropriate selective medium comprising lactose.

### Example 15. Materials and Methods Chlamydomonas reinhardtii

### Media

C. reinhardtii cells were cultured in Tris-acetate-phosphate (TAP) medium (pH 7.0). The TAP medium uses a 1000× stock Hutner's trace element mix. Hutner's trace element mix consisted of 50 g/L Na₂EDTA·H₂O (Titriplex III), 22 g/L ZnSO₄·7H₂O, 11.4 g/L H₃BO₃, 5 g/L MnCl₂·4H₂O, 5 g/L FeSO₄·7H₂O, 1.6 g/L CoCl₂·6H₂O, 1.6 g/L CuSO₄·5H₂O and 1.1 g/L (NH₄)₆MoO₃.

The TAP medium contained 2.42 g/L Tris (tris(hydroxymethyl)aminomethane), 25 mg/L salt stock solution, 0.108 g/L K₂HPO₄, 0.054 g/L KH₂PO₄ and 1.0 mL/L glacial acetic acid. The salt stock solution consisted of 15 g/L NH₄Cl, 4 g/L MgSO₄·7H₂O and 2 g/L CaCl₂·2H₂O. As precursor for saccharide synthesis, precursors like e.g. galactose, glucose, fructose, fucose, GlcNAc could be added. Medium was sterilized by autoclaving (121° C., 21 min). For stock cultures on agar slants TAP medium was used containing 1% agar (of purified high strength, 1000 g/cm2).

### Strains, Plasmids and Mutations

C. reinhardtii wild-type strains 21 gr (CC-1690, wild-type, mt+), 6145C (CC-1691, wild-type, mt−), CC-125 (137c, wild-type, mt+), CC-124 (137c, wild-type, mt−) as available from Chlamydomonas Resource Center (https://www.chlamycollection.org), University of Minnesota, U.S.A.

Expression plasmids originated from pSl103, as available from Chlamydomonas Resource Center. Cloning can be performed using Gibson Assembly, Golden Gate assembly, Cliva assembly, LCR or restriction ligation. Suitable promoters for (heterologous) gene expression can be derived from e.g. Scranton et al. (Algal Res. 2016, 15: 135-142). Targeted gene modification (like gene knock-out or gene replacement) can be carried using the Crispr-Cas technology as described e.g. by Jiang et al. (Eukaryotic Cell 2014, 13(11): 1465-1469).

Transformation via electroporation was performed as described by Wang et al. (Biosci. Rep. 2019, 39: BSR2018210). Cells were grown in liquid TAP medium under constant aeration and continuous light with a light intensity of 8000 Lx until the cell density reached 1.0-2.0×10⁷ cells/mL. Then, the cells were inoculated into fresh liquid TAP medium in a concentration of 1.0×10⁶ cells/mL and grown under continuous light for 18-20 h until the cell density reached 4.0×10⁶ cells/mL. Next, cells were collected by centrifugation at 1250 g for 5 min at room temperature, washed and resuspended with pre-chilled liquid TAP medium containing 60 mM sorbitol (Sigma, U.S.A.), and iced for 10 min. Then, 250 μL of cell suspension (corresponding to 5.0×10⁷ cells) were placed into a pre-chilled 0.4 cm electroporation cuvette with 100 ng plasmid DNA (400 ng/mL). Electroporation was performed with 6 pulses of 500 V each having a pulse length of 4 ms and pulse interval time of 100 ms using a BTX ECM830 electroporation apparatus (1575Ω, 50 μFD). After electroporation, the cuvette was immediately placed on ice for 10 min. Finally, the cell suspension was transferred into a 50 mL conical centrifuge tube containing 10 mL of fresh liquid TAP medium with 60 mM sorbitol for overnight recovery at dim light by slowly shaking. After overnight recovery, cells were recollected and plated with starch embedding method onto selective 1.5% (w/v) agar-TAP plates containing ampicillin (100 mg/L) or chloramphenicol (100 mg/L). Plates were then incubated at 23+−0.5° C. under continuous illumination with a light intensity of 8000 Lx. Cells were analysed 5-7 days later.

In an example for production of UDP-galactose, C. reinhardtii cells were modified with transcriptional units comprising the gene encoding the galactokinase from Arabidopsis thaliana (KIN, UniProt ID Q9SEE5) and the gene encoding the UDP-sugar pyrophosphorylase (USP) from A. thaliana (UniProt ID Q9C511).

In an example for LN3 production, C. reinhardtii cells were modified with a constitutive transcriptional unit comprising a galactoside beta-1,3-N-acetylglucosaminyltransferase like e.g. IgtA from N. meningitidis (UniProt ID Q9JXQ6). In an example for LNnT production, the LN3 producing strain was further modified with a constitutive transcriptional unit comprising an N-acetylglucosamine beta-1,4-galactosyltransferase like e.g. IgtB from N. meningitidis (UniProt ID Q51116).

In an example for CMP-sialic acid synthesis, C. reinhardtii cells were modified with constitutive transcriptional units for a UDP-N-acetylglucosamine-2-epimerase/N-acetylmannosamine kinase like e.g. GNE from Homo sapiens (UniProt ID Q9Y223) or a mutant form of the human GNE polypeptide comprising the R263L mutation, an N-acylneuraminate-9-phosphate synthetase like e.g. NANS from Homo sapiens (UniProt ID Q9NR45) and an N-acylneuraminate cytidylyltransferase like e.g. CMAS from Homo sapiens (UniProt ID Q8NFW8). In an example for production of sialylated oligosaccharides, C. reinhardtii cells are modified with a CMP-sialic acid transporter like e.g. CST from Mus musculus (UniProt ID Q61420), and a beta-galactoside alpha-2,6-sialyltransferase like e.g. PdST6 from Photobacterium damselae (UniProt ID 066375) or a PdST6-like polypeptide consisting of amino acid residues 108 to 497 of UniProt ID O66375 having beta-galactoside alpha-2,6-sialyltransferase activity or the polypeptide with SEQ ID NO 01.

### Heterologous and Homologous Expression

Genes that needed to be expressed, be it from a plasmid or from the genome were synthetically synthetized with one of the following companies: DNA2.0, Gen9, Twist Biosciences or IDT.

Expression could be further facilitated by optimizing the codon usage to the codon usage of the expression host. Genes were optimized using the tools of the supplier.

### Cultivation Conditions

Cells of C. reinhardtii were cultured in selective TAP-agar plates at 23+/−0.5° C. under 14/10 h light/dark cycles with a light intensity of 8000 Lx. Cells were analysed after 5 to 7 days of cultivation.

For high-density cultures, cells could be cultivated in closed systems like e.g. vertical or horizontal tube photobioreactors, stirred tank photobioreactors or flat panel photobioreactors as described by Chen et al. (Bioresour. Technol. 2011, 102: 71-81) and Johnson et al. (Biotechnol. Prog. 2018, 34: 811-827).

### Example 16. Production of LSTc with Modified C. reinhardtii Cells

C. reinhardtii cells are engineered as described in Example 15 for production of UDP-Gal with genomic knock-ins of constitutive transcriptional units comprising the galactokinase from A. thaliana (KIN, UniProt ID Q9SEE5) and the UDP-sugar pyrophosphorylase (USP) from A. thaliana (UniProt ID Q9C511). In a next step, the engineered cells are modified for CMP-sialic acid synthesis with genomic knock-ins of constitutive transcriptional units comprising a mutant form of the UDP-N-acetylglucosamine-2-epimerase/N-acetylmannosamine kinase GNE from Homo sapiens (UniProt ID Q9Y223) differing from the native polypeptide with a R263L mutation, the N-acylneuraminate-9-phosphate synthetase NANS from Homo sapiens (UniProt ID Q9NR45), the N-acylneuraminate cytidylyltransferase CMAS from Homo sapiens (UniProt ID Q8NFW8) and the CMP-sialic acid transporter CST from Mus musculus (UniProt ID Q61420). In a final step, the engineered cells are modified with an expression plasmid comprising constitutive transcriptional units comprising the alpha-2,6-sialyltransferase with SEQ ID NO 01, the galactoside beta-1,3-N-acetylglucosaminyltransferase IgtA from N. meningitis (UniProt ID Q9JXQ6) and the N-acetylglucosamine beta-1,4-galactosyltransferase IgtB from N. meningitidis (UniProt ID Q51116). The novel strain is evaluated for production of LSTc in a cultivation experiment on TAP-agar plates comprising galactose, glucose and GlcNAc as precursors according to the culture conditions provided in Example 15. After 5 days of incubation, the cells are harvested, and the saccharide production is analysed on UPLC.

### Example 17. Materials and Methods Animal Cells

Isolation of Mesenchymal Stem Cells from Adipose Tissue of Different Animals

Fresh adipose tissue is obtained from slaughterhouses (e.g. cattle, pigs, sheep, chicken, ducks, catfish, snake, frogs) or liposuction (e.g. in case of humans, after informed consent) and kept in phosphate buffer saline supplemented with antibiotics. Enzymatic digestion of the adipose tissue is performed followed by centrifugation to isolate mesenchymal stem cells. The isolated mesenchymal stem cells are transferred to cell culture flasks and grown under standard growth conditions, e.g. 37° C., 5% C02. The initial culture medium includes DMEM-F12, RPMI, and Alpha-MEM medium (supplemented with 15% foetal bovine serum), and 1% antibiotics. The culture medium is subsequently replaced with 10% FBS (foetal bovine serum)-supplemented media after the first passage. For example, Ahmad and Shakoori (2013, Stem Cell Regen. Med. 9(2): 29-36), which is incorporated herein by reference in its entirety for all purposes, describes certain variation(s) of the method(s) described herein in this example.

Isolation of Mesenchymal Stem Cells from Milk

This example illustrates isolation of mesenchymal stem cells from milk collected under aseptic conditions from human or any other mammal(s) such as described herein. An equal volume of phosphate buffer saline is added to diluted milk, followed by centrifugation for 20 min. The cell pellet is washed thrice with phosphate buffer saline and cells are seeded in cell culture flasks in DMEM-F12, RPMI, and Alpha-MEM medium supplemented with 10% foetal bovine serum and 1% antibiotics under standard culture conditions. For example, Hassiotou et al. (2012, Stem Cells. 30(10): 2164-2174), which is incorporated herein by reference in its entirety for all purposes, describes certain variation(s) of the method(s) described herein in this example.

### Differentiation of Stem Cells Using 2D and 3D Culture Systems

The mesenchymal cells isolated from adipose tissue of different animals or from milk as described above can be differentiated into mammary-like epithelial and luminal cells in 2D and 3D culture systems. See, for example, Huynh et al. 1991. Exp Cell Res. 197(2): 191-199; Gibson et al. 1991, In Vitro Cell Dev Biol Anim. 27(7): 585-594; Blatchford et al. 1999; Animal Cell Technology’: Basic & Applied Aspects, Springer, Dordrecht. 141-145; Williams et al. 2009, Breast Cancer Res 11(3): 26-43; and Arevalo et al. 2015, Am J Physiol Cell Physiol. 310(5): C348-C356; each of which is incorporated herein by reference in their entireties for all purposes.

For 2D culture, the isolated cells were initially seeded in culture plates in growth media supplemented with 10 ng/mL epithelial growth factor and 5 μg/mL insulin. At confluence, cells were fed with growth medium supplemented with 2% fetal bovine serum, 1% penicillin-streptomycin (100 U/mL penicillin, 100 μg/mL streptomycin), and 5 μg/mL insulin for 48 h. To induce differentiation, the cells were fed with complete growth medium containing 5 μg/mL insulin, 1 μg/mL hydrocortisone, 0.65 ng/mL triiodothyronine, 100 nM dexamethasone, and 1 μg/mL prolactin. After 24 h, serum is removed from the complete induction medium.

For 3D culture, the isolated cells were trypsinized and cultured in Matrigel, hyaluronic acid, or ultra-low attachment surface culture plates for six days and induced to differentiate and lactate by adding growth media supplemented with 10 ng/mL epithelial growth factor and 5 μg/mL insulin. At confluence, cells were fed with growth medium supplemented with 2% foetal bovine serum, 1% penicillin-streptomycin (100 U/mL penicillin, 100 μg/mL streptomycin), and 5 μg/mL insulin for 48 h. To induce differentiation, the cells were fed with complete growth medium containing 5 μg/mL insulin, 1 μg/mL hydrocortisone, 0.65 ng/mL triiodothyronine, 100 nM dexamethasone, and 1 μg/mL prolactin. After 24 h, serum is removed from the complete induction medium.

### Method of Making Mammary-Like Cells

In a next step, the cells are brought to induced pluripotency by reprogramming with viral vectors encoding for Oct4, Sox2, Klf4, and c-Myc. The resultant reprogrammed cells are then cultured in Mammocult media (available from Stem Cell Technologies), or mammary cell enrichment media (DMEM, 3% FBS, estrogen, progesterone, heparin, hydrocortisone, insulin, EGF) to make them mammary-like, from which expression of select milk components can be induced. Alternatively, epigenetic remodelling is performed using remodelling systems such as CRISPR/Cas9, to activate select genes of interest, such as casein, a-lactalbumin to be constitutively on, to allow for the expression of their respective proteins, and/or to down-regulate and/or knock-out select endogenous genes as described e.g. in WO21067641, which is incorporated herein by reference in its entirety for all purposes.

### Cultivation

Completed growth media includes high glucose DMEM/F12, 10% FBS, 1% NEAA, 1% pen/strep, 1% ITS-X, 1% F-Glu, 10 ng/mL EGF, and 5 μg/mL hydrocortisone. Completed lactation media includes high glucose DMEM/F12, 1% NEAA, 1% pen/strep, 1% ITS-X, 1% F-Glu, 10 ng/mL EGF, 5 μg/mL hydrocortisone, and 1 μg/mL prolactin (5 ug/mL in Hyunh 1991). Cells are seeded at a density of 20,000 cells/cm2 onto collagen coated flasks in completed growth media and left to adhere and expand for 48 hours in completed growth media, after which the media is switched out for completed lactation media. Upon exposure to the lactation media, the cells start to differentiate and stop growing. Within about a week, the cells start secreting lactation product(s) such as milk lipids, lactose, casein and whey into the media. A desired concentration of the lactation media can be achieved by concentration or dilution by ultrafiltration. A desired salt balance of the lactation media can be achieved by dialysis, for example, to remove unwanted metabolic products from the media. Hormones and other growth factors used can be selectively extracted by resin purification, for example the use of nickel resins to remove His-tagged growth factors, to further reduce the levels of contaminants in the lactated product.

### Example 18. Production of LSTc in a Non-Mammary Adult Stem Cell

Isolated mesenchymal cells and re-programmed into mammary-like cells as described in Example 17 are modified via CRISPR-CAS to express the GlcN6P synthase from Homo sapiens (UniProt ID Q06210), the glucosamine 6-phosphate N-acetyltransferase from Homo sapiens (UniProt ID Q96EK6), the phosphoacetylglucosamine mutase from Homo sapiens (UniProt ID 095394), the UDP-N-acetylhexosamine pyrophosphorylase from Homo sapiens (UniProt ID Q16222), the galactoside beta-1,3-N-acetylglucosaminyltransferase LgtA from N. meningitidis (UniProt ID Q9JXQ6), the N-acetylglucosamine beta-1,4-galactosyltransferase IgtB from N. meningitidis (UniProt ID Q51116), the N-acylneuraminate cytidylyltransferases neuA from Mus musculus (UniProt ID Q99KK2) and the alpha-2,6-sialyltransferase with SEQ ID NO 01. Cells are seeded at a density of 20,000 cells/cm2 onto collagen coated flasks in completed growth media and left to adhere and expand for 48 hours in completed growth media, after which the media is switched out for completed lactation media for about 7 days. After cultivation as described in Example 17, cells are subjected to UPLC to analyse for production of LSTc.

1. A method of producing a 6′sialylated oligosaccharide, the method comprising: contacting an alpha-2,6-sialyltransferase with a mixture comprising a donor comprising a sialic acid residue, and an acceptor selected from an oligosaccharide or a disaccharide, under conditions where the alpha-2,6-sialyltransferase catalyzes the transfer of a sialic acid residue from the donor to the acceptor, thereby producing the 6′sialylated oligosaccharide, wherein the alpha-2,6-sialyltransferase is selected from the group consisting of
i) an alpha-2,6-sialyltransferase having alpha-2,6-sialyltransferase activity on the terminal galactose (Gal) residue of lacto-N-neotetraose (LNnT, Gal-b1,4-GlcNAc-b1,3-Gal-b1,4-Glc) and comprising an amino acid sequence that is at least 60.0% identical over a stretch of at least 100 amino acid residues to the amino acid sequence as represented by SEQ ID NO: 1;
ii) an alpha-2,6-sialyltransferase having alpha-2,6-sialyltransferase activity on the terminal galactose (Gal) residue of lacto-N-neotetraose (LNnT, Gal-b1,4-GlcNAc-b1,3-Gal- b1,4-Glc) and comprising an amino acid sequence that is at least 60.0% identical to the amino acid sequence as represented by SEQ ID NO: 1 over a stretch of at least 100 amino acid residues;
iii) an alpha-2,6-sialyltransferase having alpha-2,6-sialyltransferase activity on the terminal galactose (Gal) residue of lacto-N-neotetraose (LNnT, Gal-b1,4-GlcNAc-b1,3-Gal-b1,4-Glc) and comprising an amino acid sequence that is at least 60.0% identical to the full-length amino acid sequence as represented by SEQ ID NO: 1; and
iv) an alpha-2,6-sialyltransferase having alpha-2,6-sialyltransferase activity on the terminal galactose (Gal) residue of lacto-N-neotetraose (LNnT, Gal-b1,4-GlcNAc-b1,3-Gal-b1,4-Glc) and wherein the alpha-2,6-sialyltransferase comprises an amino acid sequence as represented by SEQ ID NO: 1.

2. A method of producing a 6′sialylated oligosaccharide, the method comprising:
a) providing
i. CMP-sialic acid,
ii. an acceptor, and
iii. a sialyltransferase,
b) contacting the sialyltransferase and CMP-sialic acid with the acceptor, under conditions where the sialyltransferase catalyzes the transfer of a sialic acid residue from the CMP-sialic acid to the acceptor resulting in the production of a 6′sialylated oligosaccharide,
c) optionally, separating the produced 6′sialylated oligosaccharide; wherein the sialyltransferase is an alpha-2,6-sialyltransferase selected from the group consisting of
i) an alpha-2,6-sialyltransferase having alpha-2,6-sialyltransferase activity on the terminal galactose (Gal) residue of lacto-N-neotetraose (LNnT, Gal-b1,4-GlcNAc-b1,3-Gal-b1,4-Glc) and comprising an amino acid sequence that is at least 60.0% identical over a stretch of at least 100 amino acid residues to the amino acid sequence as represented by SEQ ID NO: 1;
ii) an alpha-2,6-sialyltransferase having alpha-2,6-sialyltransferase activity on the terminal galactose (Gal) residue of lacto-N-neotetraose (LNnT, Gal-b1,4-GlcNAc-b1,3-Gal-b1,4-Glc) and comprising an amino acid sequence that is at least 60.0% identical to the amino acid sequence as represented by SEQ ID NO: 1 over a stretch of at least 100 amino acid residues;
iii) an alpha-2,6-sialyltransferase having alpha-2,6-sialyltransferase activity on the terminal galactose (Gal) residue of lacto-N-neotetraose (LNnT, Gal-b1,4-GlcNAc-b1,3-Gal-b1,4-Glc) and comprising an amino acid sequence that is at least 60.0% identical to the full-length amino acid sequence as represented by SEQ ID NO: 1; or
iv) an alpha-2,6-sialyltransferase having alpha-2,6-sialyltransferase activity on the terminal galactose (Gal) residue of lacto-N-neotetraose (LNnT, Gal-b1,4-GlcNAc-b1,3-Gal-b1,4-Glc) and wherein the alpha-2,6-sialyltransferase comprises an amino acid sequence as represented by SEQ ID NO: 1.

3. The method according to claim 1, the method comprising: contacting a cell extract comprising the alpha-2,6-sialyltransferase with a mixture comprising a donor comprising a sialic acid residue, and an acceptor comprising an oligosaccharide or disaccharide, under conditions wherein the alpha-2,6-sialyltransferase catalyzes the transfer of a sialic acid residue from the donor to the acceptor, thereby producing the 6′sialylated oligosaccharide.

4. The method according to claim 1, wherein the 6′sialylated oligosaccharide is produced in a cell-free system.

5. A method of producing a 6′sialylated oligosaccharide, the method comprising the steps of:
i. providing a cell expressing an alpha-2,6-sialyltransferase,
ii. providing CMP-sialic acid, wherein, optionally, the CMP-sialic acid is produced by the cell, and
iii. providing an oligosaccharide or disaccharide, wherein, optionally, the oligosaccharide or disaccharide is produced by the cell, and
iv. cultivating and/or incubating the cell under conditions permissive to express the alpha-2,6-sialyltransferase, optionally permissive to produce the CMP-sialic acid and/or the oligosaccharide or disaccharide,
v. optionally, separating the 6′sialylated oligosaccharide from the cultivation or incubation
wherein the alpha-2,6-sialyltransferase is selected from the group consisting of
i) an alpha-2,6-sialyltransferase having alpha-2,6-sialyltransferase activity on the terminal galactose (Gal) residue of lacto-N-neotetraose (LNnT, Gal-b1,4-GlcNAc-b1,3-Gal-b1,4-Glc) and comprising an amino acid sequence that is at least 60.0% identical over a stretch of at least 100 amino acid residues to the amino acid sequence as represented by SEQ ID NO:1;
ii) an alpha-2,6-sialyltransferase having alpha-2,6-sialyltransferase activity on the terminal galactose (Gal) residue of lacto-N-neotetraose (LNnT, Gal-b1,4-GlcNAc-b1,3-Gal-b1,4-Glc) and comprising an amino acid sequence that is at least 60.0% identical to the amino acid sequence as represented by SEQ ID NO:1 over a stretch of at least 100 amino acid residues;
iii) an alpha-2,6-sialyltransferase having alpha-2,6-sialyltransferase activity on the terminal galactose (Gal) residue of lacto-N-neotetraose (LNnT, Gal-b1,4-GlcNAc-b1,3-Gal-b1,4-Glc) and comprising an amino acid sequence that is at least 60.0% identical to the full-length amino acid sequence as represented by SEQ ID NO:1; or
iv) an alpha-2,6-sialyltransferase having alpha-2,6-sialyltransferase activity on the terminal galactose (Gal) residue of lacto-N-neotetraose (LNnT, Gal-b1,4-GlcNAc-b1,3-Gal-b1,4-Glc) and wherein the alpha-2,6-sialyltransferase comprises an amino acid sequence as represented by SEQ ID NO:1.

6. The method according to claim 5, wherein the cell is a metabolically engineered cell.

7. The method according to claim 5,
wherein the cultivation medium contains at least one carbon source selected from the group consisting of glucose, fructose, sucrose, and glycerol.

8. The method according to claim 1, wherein the cultivation or incubation medium contains at least one compound selected from the group consisting of lactose, galactose, lacto-N-neotetraose (LNnT), UDP-galactose (UDP-Gal), UDP-N-acetylglucosamine (UDP-GlcNAc), sialic acid and CMP-sialic acid.

9. The method according to claim 1, wherein the 6′sialylated oligosaccharide is recovered from the cultivation or incubation medium and/or the cell.

10. The method according to claim 1, the method comprising:
i) utilizing a cultivation or incubation medium comprising at least one precursor and/or acceptor for the production of the 6′sialylated oligosaccharide and/or
ii) adding to the cultivation or incubation medium at least one precursor and/or acceptor feed for the production of the 6′sialylated oligosaccharide.

11. The method according to claim 1, wherein the method comprises at least one of the following:
i) utilizing a cultivation or incubation medium comprising at least one precursor and/or acceptor;
ii) adding to the cultivation or incubation medium in a reactor or incubator at least one precursor and/or acceptor feed wherein the total reactor or incubator volume ranges from 250 mL to 10,000 m³ (cubic meter);
iii) adding to the cultivation or incubation medium in a reactor or incubator at least one precursor and/or acceptor feed wherein the total reactor or incubator volume ranges from 250 mL to 10,000 m³ in a continuous manner;
iv) adding at least one precursor and/or acceptor feed in a continuous manner to the cultivation or incubation medium over the course of at least 1 day by means of a precursor and/or acceptor feeding solution;
v) adding at least one precursor and/or acceptor feed in a continuous manner to the cultivation or incubation medium over the course of at least 1 day by means of a precursor and/or acceptor feeding solution and wherein the concentration of the precursor and/or acceptor feeding solution is 50 to 600 g/L;
the method resulting in the 6′sialylated oligosaccharide with a concentration of at least 50 g/L in the final volume of the cultivation or incubation.

12. The method according to claim 1, the method comprising at least one of the following:
i) utilizing a cultivation or incubation medium comprising at least 50 grams of precursor per liter of initial reactor or incubator volume wherein the reactor or incubator volume ranges from 250 mL to 10,000 m3 (cubic meter);
ii) utilizing a cultivation or incubation medium comprising at least 50 grams of acceptor per liter of initial reactor or incubator volume wherein the reactor or incubator volume ranges from 250 mL to 10,000 m3 (cubic meter);
iii) adding to the cultivation or incubation medium in a reactor or incubator a precursor feed comprising at least 50 grams of precursor per liter of initial reactor or incubator volume wherein the reactor or incubator volume ranges from 250 mL to 10,000 m3 (cubic meter) in a continuous manner;
iv) adding to the cultivation or incubation medium in a reactor or incubator an precursor feed comprising at least 50 grams of precursor per liter of initial reactor or incubator volume wherein the reactor or incubator volume ranges from 250 mL to 10,000 m³ (cubic meter) in a continuous manner so that the final volume of the cultivation or incubation medium is not more than three-fold of the volume of the cultivation or incubation medium before the addition of the precursor feed;
v) adding to the cultivation or incubation medium in a reactor or incubator a acceptor feed comprising at least 50 grams of acceptor liter of initial reactor or incubator volume wherein the reactor or incubator volume ranges from 250 mL to 10,000 m³ (cubic meter) in a continuous manner
vi) adding to the cultivation or incubation medium in a reactor or incubator an acceptor feed comprising at least 50 grams of acceptor per liter of initial reactor or incubator volume wherein the reactor or incubator volume ranges from 250 mL to 10,000 m³ (cubic meter) in a continuous manner so that the final volume of the cultivation or incubation medium is not more than three-fold of the volume of the cultivation or incubation medium before the addition of the acceptor feed;
vii) adding to the cultivation or incubation medium a precursor feed comprising at least 50 grams of precursor per liter of initial reactor or incubator volume wherein the total reactor or incubator volume ranges from 250 mL to 10,000 m³ (cubic meter), wherein the pH of the precursor feed is set between 2.0 and 10.0;
viii) adding to the cultivation or incubation medium a precursor feed comprising at least 50 150 grams of precursor per liter of initial reactor or incubator volume wherein the reactor or incubator volume ranges from 250 mL to 10,000 m³ (cubic meter), wherein the temperature of the precursor feed is kept between 20° C. and 80° C.;
ix) adding to the cultivation or incubation medium an acceptor feed comprising at least 50 grams of acceptor liter of initial reactor or incubator volume wherein the total reactor or incubator volume ranges from 250 mL to 10,000 m³ (cubic meter), wherein the pH of the acceptor feed is set between 2.0 and 10.0;
x) adding to the cultivation or incubation medium an acceptor feed comprising at least 50 150 grams of acceptor per liter of initial reactor or incubator volume wherein the total reactor or incubator volume ranges from 250 mL to 10,000 m³ (cubic meter), wherein the temperature of the acceptor feed is kept between 20° C. and 80° C.;
xi) adding a precursor and/or acceptor feed in a continuous manner to the cultivation or incubation medium over the course of at least 1 day by means of a precursor and/or acceptor feeding solution;
xii) adding a precursor feed in a continuous manner to the cultivation or incubation medium over the course of at least 1 day, by means of a precursor feeding solution and wherein the concentration of the precursor feeding solution is 50 to 600 g/L; and wherein the pH of the precursor feeding solution is set between 2.0 and 10.0 and wherein the temperature of the precursor feeding solution is kept between 20° C. and 80° C.;
xiii) adding an acceptor feed in a continuous manner to the cultivation or incubation medium over the course of at least 1 day, by means of an acceptor feeding solution and wherein the concentration of the acceptor feeding solution is 50 to 600 g/L; and wherein the pH of the acceptor feeding solution is set between 2.0 and 10.0 and wherein the temperature of the acceptor feeding solution is kept between 20° C. and 80° C.;
the method resulting in the 6′sialylated oligosaccharide with a concentration of at least 50 g/L in the final volume of the cultivation or incubation.

13. The method according to claim 1, wherein the 6′sialylated oligosaccharide is LSTc (Neu5Ac-a2,6-Gal-b1,4-GlcNAc-b1,3-Gal-b1,4-Glc) and the acceptor is lacto-N-neotetraose (LNnT, Gal-b1,4-GlcNAc-b1,3-Gal-b1,4-Glc).

14. A metabolically engineered cell for the production of a 6′sialylated oligosaccharide, preferably LSTc (Neu5Ac-a2,6-Gal-b1,4-GlcNAc-b1,3-Gal-b1,4-Glc), wherein the cell has been metabolically engineered to possess an alpha-2,6-sialyltransferase, wherein a non-metabolically engineered cell does not comprise an alpha-2,6-sialyltransferase, wherein the alpha-2,6-sialyltransferase is selected from the group consisting of
i) alpha-2,6-sialyltransferase having alpha-2,6-sialyltransferase activity on the terminal galactose (Gal) residue of lacto-N-neotetraose (LNnT, Gal-b1,4-GlcNAc-b1,3-Gal-b1,4-Glc) and comprising an amino acid sequence that is at least 60.0% identical over a stretch of at least 100 amino acid residues to the amino acid sequence as represented by SEQ ID NO:1;
ii) an alpha-2,6-sialyltransferase having alpha-2,6-sialyltransferase activity on the terminal Gal residue of LNnT and comprising an amino acid sequence that is at least 60.0% identical to the amino acid sequence as represented by SEQ ID NO:1 over a stretch of at least 150 amino acid residues;
iii) an alpha-2,6-sialyltransferase having alpha-2,6-sialyltransferase activity on the terminal Gal residue of LNnT and comprising an amino acid sequence that is at least 60.0% identical to the full-length amino acid sequence as represented by SEQ ID NO:1; and
iv) an alpha-2,6-sialyltransferase having alpha-2,6-sialyltransferase activity on the terminal Gal residue of LNnT and wherein the alpha-2,6-sialyltransferase comprises SEQ ID NO:1.

15. Cell according to claim 14, wherein the cell contains a nucleic acid molecule which comprises a polynucleotide sequence which encodes the alpha-2,6-sialyltransferase.

16. Cell according to claim 14, wherein the cell is a bacterium, a fungus, a yeast, a plant cell, an animal cell, or a protozoan cell.

17. The cell of claim 14, wherein the cell is a metabolically engineered to comprise at least one gene encoding a polypeptide involved in a pathway for production of a sialylated di- and/or oligosaccharide.

18. The cell of claim 14, wherein the cell comprises a nucleic acid molecule comprising a polynucleotide sequence encoding the alpha-2,6-sialyltransferase and operably linked to control sequences recognized by the cell, wherein the polynucleotide sequence is foreign to the cell, the polynucleotide sequence further i) being integrated in the genome of the cell and/or ii) presented to the cell on a vector.

19. The cell of claim 14, wherein the cell comprises a catabolic pathway for selected mono-, di- or oligosaccharides which is at least partially inactivated, the mono-, di-, or oligosaccharides being involved in and/or required for the synthesis of a 6′sialylated oligosaccharide.

20. A vector comprising a nucleic acid molecule which encodes an alpha-2,6-sialyltransferase selected from the group consisting of
i) an alpha-2,6-sialyltransferase having alpha-2,6-sialyltransferase activity on the terminal galactose (Gal) residue of lacto-N-neotetraose (LNnT, Gal-b1,4-GlcNAc-b1,3-Gal-b1,4-Glc) and comprising an amino acid sequence that is at least 60.0% identical over a stretch of at least 150 amino acid residues to the amino acid sequence as represented by SEQ ID NO:1;
ii) an alpha-2,6-sialyltransferase having alpha-2,6-sialyltransferase activity on the terminal (Gal residue of LNnT and comprising an amino acid sequence that is at least 80.0% identical to the amino acid sequence as represented by SEQ ID NO:1 over a stretch of at least 100 amino acid residues;
iii) an alpha-2,6-sialyltransferase having alpha-2,6-sialyltransferase activity on the terminal Gal residue of LNnT and comprising an amino acid sequence that is at least at least 85.0% identical to the full-length amino acid sequence of SEQ ID NO:1; or
iv) an alpha-2,6-sialyltransferase having alpha-2,6-sialyltransferase activity on the terminal Gal residue of LNnT and wherein the alpha-2,6-sialyltransferase comprises an amino acid sequence as represented by SEQ ID NO:1.

21. (canceled)

22. A method of producing a 6′sialylated oligosaccharide, the method comprising:
culturing the cell of claim 14 to produce the 6′sialylated oligosaccharide.

23. The method according to claim 1, wherein the 6′sialylated oligosaccharide is Neu5Ac-a2,6-Gal-b1,4-GlcNAc-b1,3-Gal-b1,4-Glc (LSTc).

24. (canceled)

25. A method of producing a 6′sialylated oligosaccharide the method comprising:
culturing a cell comprising the vector of claim 20 to produce the 6′sialylated oligosaccharide.

26. A method of producing Neu5Ac-α2,6-Gal-b1,4-GlcNAc-b1,3-Gal-b1,4-Glc (LSTc) utilizing an α-2,6-sialyltransferase in a cell, wherein the α-2,6-sialyltransferase is selected from the group consisting of
i) an α-2,6-sialyltransferase having α-2,6-sialyltransferase activity on the terminal galactose (Gal) residue of lacto-N-neotetraose (LNnT, Gal-b1,4-GlcNAc-b1,3-Gal-b1,4-Glc) and comprising an amino acid sequence that is at least 60.0% identical over a stretch of at least 150 amino acid residues to the amino acid sequence as represented by SEQ ID NO:1;
ii) an α-2,6-sialyltransferase having α-2,6-sialyltransferase activity on the terminal Gal residue of LNnT and comprising an amino acid sequence that is at least 80.0% identical to the amino acid sequence as represented by SEQ ID NO:1 over a stretch of at least 180 amino acid residues;
iii) an α-2,6-sialyltransferase having α-2,6-sialyltransferase activity on the terminal Gal residue of LNnT and comprising an amino acid sequence that is at least 90.0% identical to the full-length amino acid of SEQ ID NO:1; and
iv) an α-2,6-sialyltransferase having α-2,6-sialyltransferase activity on the terminal Gal residue of LNnT and wherein the α-2,6-sialyltransferase comprises SEQ ID NO:1, wherein the method comprises using the cell to produce LSTc.

27. A method of producing Neu5Ac-α2,6-Gal-b1,4-GlcNAc-b1,3-Gal-b1,4-Glc (LSTc) in a cell utilizing a nucleic acid molecule that encodes an α-2,6-sialyltransferase, wherein the α-2,6-sialyltransferase is selected from the group consisting of
i) an α-2,6-sialyltransferase having α-2,6-sialyltransferase activity on the terminal galactose (Gal) residue of lacto-N-neotetraose (LNnT, Gal-b1,4-GlcNAc-b1,3-Gal-b1,4-Glc) and comprising an amino acid sequence that is at least 60.0% identical over a stretch of at least 150 amino acid residues to the amino acid sequence as represented by SEQ ID NO:1;
ii) an α-2,6-sialyltransferase having α-2,6-sialyltransferase activity on the terminal Gal residue of LNnT and comprising an amino acid sequence that is at least 80.0% identical to the amino acid sequence as represented by SEQ ID NO:1 over a stretch of at least 180 amino acid residues;
iii) an α-2,6-sialyltransferase having α-2,6-sialyltransferase activity on the terminal Gal residue of LNnT and comprising an amino acid sequence that is at least 90.0% identical to the full-length amino acid sequence of SEQ ID NO:1; and
iv) an α-2,6-sialyltransferase having α-2,6-sialyltransferase activity on the terminal Gal residue of LNnT and wherein the α-2,6-sialyltransferase comprises SEQ ID NO:1,
wherein the method comprises expressing the nucleic acid molecule and using the cell to produce LSTc.
